(19) United States
(12) Patent Application Publication
(10) Pub. No.: US 2025/0290150 A1
(43) Pub. Date: Sep. 18, 2025

(54) METHODS AND SYSTEMS FOR CHARACTERIZATION, DIAGNOSIS, AND TREATMENT OF CANCER

(71) Applicants: THE REGENTS OF THE UNIVERSITY OF CALIFORNIA, Oakland, CA (US); ONTARIO INSTITUTE FOR CANCER RESEARCH (OICR), Toronto (CA)

(72) Inventors: Paul BOUTROS, Los Angeles, CA (US); Kathleen HOULAHAN, Toronto (CA)

(73) Assignees: THE REGENTS OF THE UNIVERSITY OF CALIFORNIA, Oakland, CA (US); ONTARIO INSTITUTE FOR CANCER RESEARCH (OICR), Toronto, ON (CA)

(21) Appl. No.: 18/862,388

(22) PCT Filed: May 4, 2023
(86) PCT No.: PCT/US2023/021056

§ 371 Date: Nov. 1, 2024

Related U.S. Application Data
(60) Provisional application No. 63/338,373, filed on May 4, 2022.

(51) Int. Cl. C12Q 1/6886

(52) U.S. Cl. CPC C12Q 1/6886; C12Q 2600/106; C12Q 2600/156; C12Q 2600/158

(57) ABSTRACT

Disclosed herein are methods and systems for characterization, diagnosis, and treatment of cancer. Aspects of the present disclosure are directed to methods for prediction and identification of various molecular features of cancer using analysis of germline genetic information (e.g., polymorphisms). Certain aspects pertain to identification of one or more genetic abnormalities (e.g., mutations, translocations, etc.) of a cancer in a subject following genotyping the subject as having one or more polymorphisms associated with the one or more genetic abnormalities. Also disclosed are methods for diagnosis and characterization of cancer, as well as methods for treatment of cancer having particular genetic abnormalities associated with one or more polymorphisms.

This application claims priority to U.S. Provisional Application Ser. No. 63/338,373, filed May 4, 2023, which is incorporated by reference herein in its entirety.

This invention was made with government support under CA214194 awarded by the National Institutes of Health. The government has certain rights in the invention.

### BACKGROUND

### I. Field of the Invention

Aspects of this invention relate to at least the fields of cancer biology, genetics, and medicine.

### II. Background

Cancers result from the accumulation of genomic and epigenomic aberrations, deregulating normal cellular processes^{1,2}. These aberrations arise from environmental influences, genetic susceptibility and stochastic errors³. The exact contribution of each of these three factors to the mutational landscape of any specific tumor is largely unknown, as are the set of ways in which the three factors interact.

Of these three, the influences of germline genetics on cancer incidence are particularly well-quantified. About a third of the risk of cancer diagnosis is heritable⁴. Genome-wide association studies (GWAS) have identified hundreds of specific sequence variations—predominantly single nucleotide polymorphisms (SNPs)—associated with risk of diagnosis⁵⁻⁷. The mechanisms by which these germline predisposition loci modulate risk are mostly unknown.

There exists a need for methods and systems for predicting and characterizing cancer molecular phenotypes, as well as methods for diagnosis and targeted treatment of cancer, based on germline genomic analysis.

### SUMMARY

Aspects of the present disclosure provide methods, systems, and compositions useful in characterization, diagnosis, and treatment of cancer based on germline genomic analysis. Accordingly, disclosed herein are methods for diagnosing a subject as having cancer comprising detecting the presence or absence of one or more genetic abnormalities in a subject (e.g., in tumor DNA or RNA from the subject) genotyped as having one or more polymorphisms (e.g., single nucleotide polymorphisms) associated with the one or more genetic abnormalities. Also disclosed are methods for treating a subject for cancer comprising administering an effective amount of a cancer treatment to a subject who has been determined to have the presence or absence of one or more genetic abnormalities (e.g., in tumor DNA or RNA from the subject) and also genotyped as having one or more single nucleotide polymorphisms (SNPs) associated with the one or more genetic abnormalities. Certain aspects are directed to methods of diagnosis, treatment, characterization, and analysis of prostate cancer.

Embodiments of the present disclosure include methods for cancer diagnosis, methods for cancer treatment, methods for cancer prognosis, methods for preventing cancer, methods for predicting cancer occurance, methods for predicting cancer characteristics, methods for predicting a genetic abnormality, methods for characterizing cancer, methods for identifying a subject as having cancer, methods for diagnosing a subject with prostate cancer, methods for detecting single nucleotide polymorphisms, methods for identifying a genetic abnormality, methods for genotyping a subject, and methods for evaluating a risk of developing cancer. Methods of the present disclosure can include at least 1, 2, 3, 4, or more of the following steps: obtaining a biological sample from a subject, isolating nucleic acids from a subject, sequencing nucleic acids from a subject, amplifying nucleic acids from a subject, isolating tumor DNA from a subject, sequencing tumor DNA from a subject, isolating tumor RNA from a subject, sequencing tumor RNA from a subject, detecting the presence of a genetic abnormality in a subject, genotyping a subject, detecting a single nucleotide polymorphism in a subject, sequencing germline DNA from a subject, and administering a cancer therapy to a subject. Any one or more of the preceeding steps may be excluded from certain embodiments of the disclosure.

Aspects of the disclosure are directed to a method for identifying one or more genetic abnormalities in a subject. In some embodiments, the disclosed methods comprise detecting the presence of a genetic abnormality in a subject, for example from tumor DNA or tumor RNA from a subject, who has been genotyped as having one or more SNPs associated with the genetic abnormality. Non-limiting examples of genetic abnormalities and associated SNPs contemplated herein are provided in Table 1. Such methods may be useful in, for example, detecting the presence of cancer cells in the subject. Accordingly, disclosed herein, in some embodiments, is a method for identifying one or more genetic abnormalities in a subject, the method comprising (a) detecting the presence of a TMPRSS2-ERG fusion protein in a subject genotyped as having one or more single nucleotide polymorphisms selected from the group consisting of rs111620024, rs12500426, rs7679673, rs12653946, rs2837396, and rs2839469; (b) detecting the presence of a single nucleotide variation in a UTR of FOXA1 in a subject genotyped as having one or more single nucleotide polymorphisms selected from the group consisting of rs77404504, rs848047, and rs848048; (c) measuring a reduced expression level, deletion, or translocation of TMPRSS2 in a subject genotyped as having one or more single nucleotide polymorphisms selected from the group consisting of rs11203152, rs12500426, rs12653946, rs13048402, rs2837396, and rs5759167; or (d) measuring a reduced expression level or deletion of CDKN1B in a subject genotyped as having one or more single nucleotide polymorphisms selected from the group consisting of rs12817741, rs12824766, rs141393446, rs141853059, rs57526507, and rs61915608. In some embodiments, the method comprises at least 2, at least 3, or all of (a), (b), (c), and (d). In some embodiments, the method excludes any one or more of (a), (b), (c), or (d). In some aspects, the variation in a UTR of FOXA1 is a variation in the 5′ UTR of FOXA1. In some aspects, the variation in a UTR of FOXA1 is a variation in the 3′ UTR of FOXA1.

Disclosed herein, in some aspects, is a method for identifying a TMPRSS2-ERG fusion protein in a subject comprising detecting the presence of a TMPRSS2-ERG fusion protein in a subject genotyped as having one or more single nucleotide polymorphisms selected from the group consisting of rs111620024, rs12500426, rs7679673, rs12653946, rs2837396, and rs2839469. Also disclosed herein, in some embodiments, is a method for identifying a TMPRSS2-ERG fusion protein in a subject, the method comprising (a) genotyping a subject as having one or more single nucleotide polymorphisms selected from the group consisting of rs111620024, rs12500426, rs7679673, rs12653946, rs2837396, and rs2839469; and (b) detecting the presence of a TMPRSS2-ERG fusion protein in the subject. In some embodiments, (b) is performed prior to (a). In some embodiments, (b) is performed subsequent to (a). In some embodiments, the one or more single nucleotide polymorphisms comprise rs111620024. In some embodiments, the one or more single nucleotide polymorphisms comprise rs12500426. In some embodiments, the one or more single nucleotide polymorphisms comprise rs7679673. In some embodiments, the one or more single nucleotide polymorphisms comprise rs12653946. In some embodiments, the one or more single nucleotide polymorphisms comprise rs2837396. In some embodiments, the one or more single nucleotide polymorphisms comprise rs2839469. In some embodiments, the one or more single nucleotide polymorphisms are two or more of rs111620024, rs12500426, rs7679673, rs12653946, rs2837396, and rs2839469. In some embodiments, the one or more single nucleotide polymorphisms are three or more of rs111620024, rs12500426, rs7679673, rs12653946, rs2837396, and rs2839469. In some embodiments, the one or more single nucleotide polymorphisms are four or more of rs111620024, rs12500426, rs7679673, rs12653946, rs2837396, and rs2839469. In some embodiments, the one or more single nucleotide polymorphisms are five or more of rs111620024, rs12500426, rs7679673, rs12653946, rs2837396, and rs2839469. In some embodiments, the one or more single nucleotide polymorphisms are rs111620024, rs12500426, rs7679673, rs12653946, rs2837396, and rs2839469. The one or more single nucleotide polymorphisms may be any combination of rs111620024, rs12500426, rs7679673, rs12653946, rs2837396, and rs2839469. Any one or more of rs111620024, rs12500426, rs7679673, rs12653946, rs2837396, and rs2839469 may be excluded from certain embodiments of the disclosure.

Disclosed herein, in some aspects, is a method for identifying a single nucleotide variation in a UTR of FOXA1 in a subject, the method comprising detecting the presence of a single nucleotide variation in a UTR of FOXA1 in a subject genotyped as having one or more single nucleotide polymorphisms selected from the group consisting of rs77404504, rs848047, and rs848048. The UTR may be a 5′ UTR and/or a 3′ UTR. Also disclosed herein, in some embodiments, is a method for identifying a single nucleotide variation in a UTR of FOXA1 in a subject, the method comprising (a) genotyping a subject as having one or more single nucleotide polymorphisms selected from the group consisting of rs77404504, rs848047, and rs848048; and (b) detecting the presence of a UTR of FOXA1 in the subject. The UTR may be a 5′ UTR and/or a 3′ UTR. In some embodiments, (b) is performed prior to (a). In some embodiments, (b) is performed subsequent to (a). In some embodiments, the one or more single nucleotide polymorphisms are rs77404504. In some embodiments, the one or more single nucleotide polymorphisms are rs848047. In some embodiments, disclosed the single nucleotide polymorphism is rs848048. In some embodiments, the one or more single nucleotide polymorphisms are two or more of rs77404504, rs848047, and rs848048. In some embodiments, the one or more single nucleotide polymorphisms are rs77404504, rs848047, and rs848048. The one or more single nucleotide polymorphisms may be any combination of rs77404504, rs848047, and rs848048. Any one or more of rs77404504, rs848047, and rs848048 may be excluded from certain embodiments of the disclosure.

Disclosed herein, in some aspects, is a method for assaying for TMPRSS2 in a subject, the method comprising detecting a reduced expression level, deletion, or translocation of TMPRSS2 in a subject genotyped as having one or more single nucleotide polymorphisms selected from the group consisting of rs11203152, rs12500426, rs12653946, rs13048402, rs2837396, and rs5759167. Also disclosed herein, in some embodiments, is a method for assaying for TMPRSS2 in a subject, the method comprising (a) genotyping a subject as having one or more single nucleotide polymorphisms selected from the group consisting of rs11203152, rs12500426, rs12653946, rs13048402, rs2837396, and rs5759167; and (b) detecting a reduced expression level, deletion, or translocation of TMPRSS2 in the subject. In some embodiments, (b) is performed prior to (a). In some embodiments, (b) is performed subsequent to (a). In some embodiments, the one or more single nucleotide polymorphisms comprise rs11203152. In some embodiments, the one or more single nucleotide polymorphisms comprise rs12500426. In some embodiments, the one or more single nucleotide polymorphisms comprise rs12653946. In some embodiments, the one or more single nucleotide polymorphisms comprise rs13048402. In some embodiments, the one or more single nucleotide polymorphisms comprise rs2837396. In some embodiments, the one or more single nucleotide polymorphisms comprise rs5759167. In some embodiments, the one or more single nucleotide polymorphisms are two or more of rs11203152, rs12500426, rs12653946, rs13048402, rs2837396, and rs5759167. In some embodiments, the one or more single nucleotide polymorphisms are three or more of rs11203152, rs12500426, rs12653946, rs13048402, rs2837396, and rs5759167. In some embodiments, the one or more single nucleotide polymorphisms are four or more of rs11203152, rs12500426, rs12653946, rs13048402, rs2837396, and rs5759167. In some embodiments, the one or more single nucleotide polymorphisms are five or more of rs11203152, rs12500426, rs12653946, rs13048402, rs2837396, and rs5759167. In some embodiments, the one or more single nucleotide polymorphisms are rs11203152, rs12500426, rs12653946, rs13048402, rs2837396, and rs5759167. The one or more single nucleotide polymorphisms may be any combination of rs11203152, rs12500426, rs12653946, rs13048402, rs2837396, and rs5759167. Any one or more of rs11203152, rs12500426, rs12653946, rs13048402, rs2837396, and rs5759167 may be excluded from certain embodiments of the disclosure.

Disclosed herein, in some aspects, is a method for assaying for CDKN1B in a subject, the method comprising detecting a reduced expression level or deletion of CDKN1B in a subject genotyped as having one or more single nucleotide polymorphisms selected from the group consisting of rs12817741, rs12824766, rs141393446, rs141853059, rs57526507, and rs61915608. Also disclosed herein, in some embodiments, is a method for assaying for CDKN1B in a subject, the method comprising (a) genotyping a subject as having one or more single nucleotide polymorphisms selected from the group consisting of rs12817741, rs12824766, rs141393446, rs141853059, rs57526507, and rs61915608; and (b) detecting a reduced expression level or deletion of CDKN1B in the subject. In some embodiments, (b) is performed prior to (a). In some embodiments, (b) is performed subsequent to (a). In some embodiments, the one or more single nucleotide polymorphisms comprise rs12817741. In some embodiments, the one or more single nucleotide polymorphisms comprise rs12824766. In some embodiments, the one or more single nucleotide polymorphisms comprise rs141393446. In some embodiments, the one or more single nucleotide polymorphisms comprise rs141853059. In some embodiments, the one or more single nucleotide polymorphisms comprise rs57526507. In some embodiments, the one or more single nucleotide polymorphisms comprise c. In some embodiments, the one or more single nucleotide polymorphisms are two or more of rs12817741, rs12824766, rs141393446, rs141853059, rs57526507, and rs61915608. In some embodiments, the one or more single nucleotide polymorphisms are three or more of rs12817741, rs12824766, rs141393446, rs141853059, rs57526507, and rs61915608. In some embodiments, the one or more single nucleotide polymorphisms are four or more of rs12817741, rs12824766, rs141393446, rs141853059, rs57526507, and rs61915608. In some embodiments, the one or more single nucleotide polymorphisms are five or more of rs12817741, rs12824766, rs141393446, rs141853059, rs57526507, and rs61915608. In some embodiments, the one or more single nucleotide polymorphisms are rs12817741, rs12824766, rs141393446, rs141853059, rs57526507, and rs61915608. The one or more single nucleotide polymorphisms may be any combination of rs12817741, rs12824766, rs141393446, rs141853059, rs57526507, and rs61915608. Any one or more of rs12817741, rs12824766, rs141393446, rs141853059, rs57526507, and rs61915608 may be excluded from certain embodiments of the disclosure.

In some embodiments, detecting the presence of the TMPRSS2-ERG fusion protein, detecting the presence of a single nucleotide variation in a UTR of FOXA1, detecting a reduced expression level, deletion, or translocation of TMPRSS2, and/or detecting a reduced expression level or deletion of CDKN1B comprises sequencing nucleic acids from a biological sample from the subject. The UTR may be a 5′ UTR and/or a 3′ UTR. In some embodiments, the nucleic acids are cell free DNA. In some embodiments, nucleic acids are cell free RNA. In some embodiments, the biological sample is a cell free sample. In some embodiments, the biological sample is a tissue sample. In some embodiments, the biological sample is a blood sample. In some embodiments, the biological sample is a saliva sample. In some embodiments, the biological sample is a urine sample.

Further aspects of the present disclosure are directed to a method for treating a subject for cancer. In some embodiments, the cancer is prostate cancer. In some embodiments, the method comprises administering an effective amount of a prostate cancer therapy to a subject determined to have one or more genetic abnormalities, for example from tumor DNA or tumor RNA from the subject, and has been genotyped as having one or more SNPs associated with the one or more genetic abnormalities. Non-limiting examples of genetic abnormalities and associated SNPs contemplated herein are provided in Table 1. In some embodiments, the method comprises (a) detecting a genetic abnormality in a subject (e.g., from tumor DNA or tumor RNA from the subject); (b) genotyping the subject as having one or more single nucleotide polymorphisms associated with the genetic abnormality; and (c) administering an effective amount of a prostate cancer therapy to the subject. Accordingly, disclosed herein, in some embodiments, is method for treating a subject for prostate cancer, the method comprising administering an effective amount of a prostate cancer therapy to a subject who (a) has been determined to have a TMPRSS2-ERG fusion protein and has been genotyped as having one or more single nucleotide polymorphisms selected from the group consisting of rs111620024, rs12500426, rs7679673, rs12653946, rs2837396, and rs2839469; (b) has been determined to have a single nucleotide variation in a UTR of FOXA1 and has been genotyped as having one or more single nucleotide polymorphisms selected from the group consisting of rs77404504, rs848047, and rs848048; (c) has been determined to have reduced expression, a deletion, or a translocation of TMPRSS2 and has been genotyped as having one or more single nucleotide polymorphisms selected from the group consisting of rs11203152, rs12500426, rs12653946, rs13048402, rs2837396, and rs5759167; or (d) has been determined to have reduced expression or a deletion of CDKN1B and has been genotyped as having a single nucleotide polymorphism selected from the group consisting of rs12817741, rs12824766, rs141393446, rs141853059, rs57526507, and rs61915608. In some embodiments, the method comprises at least 2, at least 3, or all of (a), (b), (c), and (d). In some embodiments, the method excludes any one or more of (a), (b), (c), or (d). The UTR may be a 5′ UTR and/or a 3′ UTR.

In some embodiments, disclosed is a method for treating a subject for prostate cancer, the method comprising administering an effective amount of a prostate cancer therapy to a subject who (a) has been determined to have a TMPRSS2-ERG fusion protein and (b) has been genotyped as having one or more single nucleotide polymorphisms selected from the group consisting of rs111620024, rs12500426, rs7679673, rs12653946, rs2837396, and rs2839469. In some embodiments, disclosed is a method for treating a subject for prostate cancer, the method comprising (a) detecting the presence of a TMPRSS2-ERG fusion protein in the subject; (b) genotyping the subject as having one or more single nucleotide polymorphisms selected from the group consisting of rs111620024, rs12500426, rs7679673, rs12653946, rs2837396, and rs2839469; and (c) administering an effective amount of a prostate cancer therapy to the subject. In some embodiments, the one or more single nucleotide polymorphisms comprise rs111620024. In some embodiments, the one or more single nucleotide polymorphisms comprise rs12500426. In some embodiments, the one or more single nucleotide polymorphisms comprise rs7679673. In some embodiments, the one or more single nucleotide polymorphisms comprise rs12653946. In some embodiments, the one or more single nucleotide polymorphisms comprise rs2837396. In some embodiments, the one or more single nucleotide polymorphisms comprise rs2839469. In some embodiments, the one or more single nucleotide polymorphisms are two or more of rs111620024, rs12500426, rs7679673, rs12653946, rs2837396, and rs2839469. In some embodiments, the one or more single nucleotide polymorphisms are three or more of rs111620024, rs12500426, rs7679673, rs12653946, rs2837396, and rs2839469. In some embodiments, the one or more single nucleotide polymorphisms are four or more of rs111620024, rs12500426, rs7679673, rs12653946, rs2837396, and rs2839469. In some embodiments, the one or more single nucleotide polymorphisms are five or more of rs111620024, rs12500426, rs7679673, rs12653946, rs2837396, and rs2839469. In some embodiments, the one or more single nucleotide polymorphisms are rs111620024, rs12500426, rs7679673, rs12653946, rs2837396, and rs2839469. The one or more single nucleotide polymorphisms may be any combination of 2, 3, 4, or 5, or all, of rs111620024, rs12500426, rs7679673, rs12653946, rs2837396, and rs2839469. Anyone or more of rs111620024, rs12500426, rs7679673, rs12653946, rs2837396, and rs2839469 may be excluded from certain embodiments of the disclosure.

In some embodiments, disclosed is a method for treating a subject for prostate cancer, the method comprising administering an effective amount of a prostate cancer therapy to a subject who (a) has been determined to have a single nucleotide variation in a UTR of FOXA1 and (b) has been genotyped as having one or more single nucleotide polymorphisms selected from the group consisting of rs77404504, rs848047, and rs848048. In some embodiments, disclosed is a method for treating a subject for prostate cancer, the method comprising (a) detecting the presence of a single nucleotide variation in a 5′ UTR of FOXA1 in the subject; (b) genotyping the subject as having one or more single nucleotide polymorphisms selected from the group consisting of rs77404504, rs848047, and rs848048; and (c) administering an effective amount of a prostate cancer therapy to the subject. The UTR may be a 5′ UTR and/or a 3′ UTR. In some embodiments, the one or more single nucleotide polymorphisms comprise rs77404504. In some embodiments, the one or more single nucleotide polymorphisms comprise rs848047. In some embodiments, the one or more single nucleotide polymorphisms comprise rs848048. In some embodiments, the one or more single nucleotide polymorphisms are two or more of rs77404504, rs848047, and rs848048. In some embodiments, the one or more single nucleotide polymorphisms are rs77404504, rs848047, and rs848048. The one or more single nucleotide polymorphisms may be any combination of 2, or all, of rs77404504, rs848047, and rs848048. Any one or more of rs77404504, rs848047, and rs848048 may be excluded from certain embodiments of the disclosure.

In some embodiments, disclosed is a method for treating a subject for prostate cancer, the method comprising administering an effective amount of a prostate cancer therapy to a subject who (a) has been determined to have a reduced expression level, deletion, or translocation of TMPRSS2 and (b) has been genotyped as having one or more single nucleotide polymorphisms selected from the group consisting of rs11203152, rs12500426, rs12653946, rs13048402, rs2837396, and rs5759167. In some embodiments, disclosed is a method for treating a subject for prostate cancer, the method comprising (a) detecting a reduced expression level, deletion, or translocation of TMPRSS2 in the subject; (b) genotyping the subject as having one or more single nucleotide polymorphisms selected from the group consisting of rs11203152, rs12500426, rs12653946, rs13048402, rs2837396, and rs5759167; and (c) administering an effective amount of a prostate cancer therapy to the subject. In some embodiments, the one or more single nucleotide polymorphisms comprise rs11203152. In some embodiments, the one or more single nucleotide polymorphisms comprise rs12500426. In some embodiments, the one or more single nucleotide polymorphisms comprise rs12653946. In some embodiments, the one or more single nucleotide polymorphisms comprise rs13048402. In some embodiments, the one or more single nucleotide polymorphisms comprise rs2837396. In some embodiments, the single nucleotide polymorphism is rs5759167. In some embodiments, the one or more single nucleotide polymorphisms are two or more of rs11203152, rs12500426, rs12653946, rs13048402, rs2837396, and rs5759167. In some embodiments, the one or more single nucleotide polymorphisms are three or more of rs11203152, rs12500426, rs12653946, rs13048402, rs2837396, and rs5759167. In some embodiments, the one or more single nucleotide polymorphisms are four or more of rs11203152, rs12500426, rs12653946, rs13048402, rs2837396, and rs5759167. In some embodiments, the one or more single nucleotide polymorphisms are five or more of rs11203152, rs12500426, rs12653946, rs13048402, rs2837396, and rs5759167. In some embodiments, the one or more single nucleotide polymorphisms are rs11203152, rs12500426, rs12653946, rs13048402, rs2837396, and rs5759167. The one or more single nucleotide polymorphisms may be any combination of 2, 3, 4, or 5, or all, of rs11203152, rs12500426, rs12653946, rs13048402, rs2837396, and rs5759167. Any one or more of rs11203152, rs12500426, rs12653946, rs13048402, rs2837396, and rs5759167 may be excluded from certain embodiments of the disclosure.

In some embodiments, disclosed is a method for treating a subject for prostate cancer, the method comprising administering an effective amount of a prostate cancer therapy to a subject who (a) has been determined to have a reduced expression or deletion of CDKN1B and (b) has been genotyped as having one or more single nucleotide polymorphisms selected from the group consisting of rs12817741, rs12824766, rs141393446, rs141853059, rs57526507, and rs61915608. In some embodiments, disclosed is a method for treating a subject for prostate cancer, the method comprising (a) detecting a reduced expression level or deletion of CDKN1B in the subject; (b) genotyping the subject as having one or more single nucleotide polymorphisms selected from the group consisting of rs12817741, rs12824766, rs141393446, rs141853059, rs57526507, and rs61915608; and (c) administering an effective amount of a prostate cancer therapy to the subject. In some embodiments, the one or more single nucleotide polymorphisms are rs12817741. In some embodiments, the one or more single nucleotide polymorphisms are rs12824766. In some embodiments, the one or more single nucleotide polymorphisms are rs141393446. In some embodiments, the one or more single nucleotide polymorphisms are rs141853059. In some embodiments, the one or more single nucleotide polymorphisms are rs57526507. In some embodiments, the one or more single nucleotide polymorphisms are rs61915608. In some embodiments, the one or more single nucleotide polymorphisms are two or more of rs12817741, rs12824766, rs141393446, rs141853059, rs57526507, and rs61915608. In some embodiments, the one or more single nucleotide polymorphisms are three or more of rs12817741, rs12824766, rs141393446, rs141853059, rs57526507, and rs61915608. In some embodiments, the one or more single nucleotide polymorphisms are four or more of rs12817741, rs12824766, rs141393446, rs141853059, rs57526507, and rs61915608. In some embodiments, the one or more single nucleotide polymorphisms are five or more of rs12817741, rs12824766, rs141393446, rs141853059, rs57526507, and rs61915608. In some embodiments, the one or more single nucleotide polymorphisms are rs12817741, rs12824766, rs141393446, rs141853059, rs57526507, and rs61915608. The one or more single nucleotide polymorphisms may be any combination of 2, 3, 4, or 5, or all, of rs12817741, rs12824766, rs141393446, rs141853059, rs57526507, and rs61915608. Any one or more of rs12817741, rs12824766, rs141393446, rs141853059, rs57526507, and rs61915608 may be excluded from certain embodiments of the disclosure.

In some embodiments, the prostate cancer therapy comprises chemotherapy, hormone therapy, radiotherapy, surgery, immunotherapy, or a combination thereof. In some embodiments, the prostate cancer therapy is local prostate cancer therapy. In some embodiments, the prostate cancer therapy is systemic prostate cancer therapy. In some embodiments, the subject was previously treated for prostate cancer. In some embodiments, the subject was determind to be resistant to the previous treatment. In some embodiments, the prostate cancer is Stage I, II (e.g., IIA or IIB), III, or IV prostate cancer. In some embodiments, the prostate cancer is recurrant prostate cancer.

“Individual, “subject,” and “patient” are used interchangeably and can refer to a human or non-human.

The term “prognosis” as used herein refers to the prediction of a clinical outcome associated with a disease subtype which is reflected by a reference profile such as a biomarker reference profile. The prognosis provides an indication of disease progression and includes an indication of likelihood of death due to cancer. The prognosis may be a prediction of metastasis, or alternatively disease recurrence. In one embodiment the clinical outcome class includes a better survival group and a worse survival group. The term “prognosing” as used herein means predicting the clinical outcome of a subject according to the subject's similarity to a reference profile or biomarker associated with the prognosis. For example, prognosing or classifying comprises a method or process of determining whether an individual has a better or worse survival outcome, or grouping individuals into a better survival group or a worse survival group, or predicting whether or not an individual will respond to therapy.

The term “genotyping” as used herein refers generally to the physical, chemical, and/or electrical determination of a sequence of a nucleic acid from a subject. In certain embodiments, genotyping comprises sequencing, nucleic acid amplification, hybridization, and/or transcription, or a combination thereof. In some embodiments, genotyping comprises determination of a sequence of a portion of germline DNA from a subject. In embodiments of the disclosure, genotyping serves to identify the presence or absence of one or more polymorphisms (e.g., SNPs) in a subject.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that embodiments described herein in the context of the term “comprising” may also be implemented in the context of the term “consisting of” or “consisting essentially of.”

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary, Detailed Description, Claims, and Brief Description of the Drawings.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1F show results from studies described in Example 1, demonstrating that risk SNPs bias somatic mutational landscape. FIG. 1A shows a schematic of dQTL detection. First, 147 SNPs from the polygenic risk score proposed by Schumacher et al.⁹ were interrogated for their association with 37 somatic drivers. Second, the inventors identified linear local dQTLs by interrogating SNPs +/−500 kbp around the driver gene. Third, the inventors identified spatial local dQTLs by interrogating SNPs that interacted with each driver gene in 3D space, outside of the linear gene region. Spatial local regions were defined using RAD21 and RNA Pol-II ChIA-PET profiling in LNCaP, Du145, VCaP and RWPE1 cell lines. Finally, the inventors identified enhancer local dQTLs by interrogating SNPs in enhancer regions that interacted with the driver gene. Enhancer regions were defined using H3K27ac HiCHIP profiling in LNCaP cell lines. All discovered dQTLs were tested for replication in four replication cohorts. FIGS. 1B-1C show that the PRS was negatively associated with PGA in both the discovery cohort (FIG. 1B) and the replication cohort (FIG. 1C). Key indicates fold change (FC) and p-value between >75% and <25% groups from Mann-Whitney test. Boxplot represents median, 0.25 and 0.75 quantiles with whiskers at 1.5× interquartile range. Green dots indicate discovery cohort while purple dots indicated replication cohort. FIGS. 1D-1E show that the PRS was negatively associated with number of somatic drivers in both the discovery (FIG. 1D) and the replication cohort (FIG. 1E). FIG. 1F shows that nine risk SNPs were involved in 11 risk dQTLs with seven somatic drivers. Dot size and colour represents odd ratio magnitude and direction while background shading represents FDR. Barplot along the top indicates the number of dQTLs per SNP while the barplot on the right indicates the number of dQTLs per somatic driver. Covariate along the bottom indicates type of somatic mutation. FIG. 1G shows the Schumacher et al. polygenic risk score is associated with a younger age at diagnosis; Kaplan Meier curves demonstrate the age of diagnosis in individuals within the top and bottom 25^{th} percentile in two independent cohorts. FIG. 1H shows a high polygenic risk score (PRS) is associated with better prognosis; in the discovery cohort a high PRS was associated with decreased risk of biochemical recurrence (BCR) and metastasis; in the replication cohort, a high PRS was associated with decreased risk of progression; scatterplot demonstrates the hazard ratio (HR) of PRS with three endpoints in two independent cohorts; PFI=progression free interval. MFS=metastasis free survival.

FIGS. 2A-2G show results from studies described in Example 1, demonstrating discovery of linear local dQTLs. FIG. 2A shows a schematic outlining linear local dQTL discovery. FIG. 2B shows a summary of 34 discovery linear local dQTLs. Dot size and colour indicates magnitude and direction of ORs between SNP, x-axis and somatic driver, y-axis. Background shading indicates p-values. Covariate on left indicates the type of somatic mutation. FIG. 2C shows a comparison of ORs in discovery, x-axis, vs replication, y-axis, cohort of 16 dQTLs, considering only unique haplotypes. Horizontal and vertical dotted lines represent OR=1 and diagonal line represents y=x. Halo around points indicates FDR<0.1 in replication cohort. FIGS. 2D-2E show contingency tables of rs11203152 associated with clonal loss of TMPRSS2 in discovery cohort (FIG. 2D) and replication (FIG. 2E) cohort. FIGS. 2F-2G show contingency tables of rs141393446 associated with clonal loss of ZNF292 in the discovery (FIG. 2F) and replication (FIG. 2G) cohorts.

FIGS. 3A-3G show results from studies described in Example 1, demonstrating discovery of spatial local dQTLs. FIG. 3A shows a schematic outlining spatial local dQTL discovery. FIG. 3B shows a summary of four discovery spatial local dQTLs. Dot size and colour indicates magnitude and direction of ORs between SNP, x-axis and somatic driver, y-axis. Background shading indicates p-values. Covariate on left indicates the type of somatic mutation. FIG. 3C shows a comparison of ORs in discovery, x-axis, vs replication, y-axis, cohort of four dQTLs. Horizontal and vertical dotted lines represent OR=1 and diagonal line represents y=x. FIGS. 3D-3E show contingency tables of rs12385878 associated with clonal loss of RB1 in discovery cohort (FIG. 3D) and replication (FIG. 3E) cohort. FIGS. 3F-3G show contingency tables of rs7320595 associated with loss of RB1 in discovery (FIG. 3F) and replication (FIG. 3G) cohort.

FIGS. 4A-4G show results from studies described in Example 1, demonstrating discovery of enhancer local dQTLs. FIG. 4A shows a schematic outlining enhancer local dQTL discovery. FIG. 4B shows a summary of 17 discovery enhancer local dQTLs. Dot size and colour indicates magnitude and direction of ORs between SNP, x-axis and somatic driver, y-axis. Background shading indicates p-values. Covariate on left indicates the type of somatic mutation. FIG. 4C shows a comparison of ORs in discovery, x-axis, vs replication, y-axis, cohort of 13 dQTLs, considering only unique haplotypes. Horizontal and vertical dotted lines represent OR=1 and diagonal line represents y=x. Halo around points indicates FDR<0.1 in replication cohort. FIGS. 4D-4E show contingency tables of rs848048 associated with SNVs in FOXA1 3′ UTR in discovery cohort (FIG. 4D) and replication (FIG. 4E) cohort. FIGS. 4F-4G show contingency tables of rs848047 associated with SNVs in 3′ UTR of FOXA1 in discovery (FIG. 4F) and replication (FIG. 4G) cohorts.

FIGS. 5A-5F show results from studies described in Example 1, demonstrating characterization of dQTLs. FIG. 5A shows a summary of all 62 dQTLs. Dot size and colour indicates magnitude and direction of ORs between SNP, x-axis and somatic driver, y-axis. Background shading indicates strategy dQTL was discovered with. Covariate on left indicates the type of somatic mutation. FIG. 5B shows a comparison of ORs in discovery, x-axis, and replication, y-axis, cohorts for 61 dQTLs (one dQTL could not be tested in replication cohort). Dot colour represents strategy used to discovery dQTL. Horizontal and vertical dotted lines represent OR=1 and diagonal line represents y=x. FIG. 5C (left panel) shows that a subset of dQTLs were associated with changes in tumor methylation. Heatmap indicates the number of methylation probes each SNP, x-axis, was associated with in the discovery and replication TCGA cohort, y-axis. The third column indicates the number of replicated meQTLs that were tumor specific. The covariate on the right indicates if the SNP is a risk SNP and what somatic driver it is associated with; (right panel) shows meta-analyses across six independent cohorts identified 11 statistically significant dQTLs (FDR<0.1); scatterplot demonstrates meta-analysis odds ratio (OR) on the x-axis and the SNP on the y-axis; colour covariate in the middle indicates the somatic driver each SNP is associated with; heatmap on the right indicates which cohorts were included in the meta-analysis for each dQTL. FIG. 5D shows that dQTL SNPs (x-axis) overlap with histone modification and transcription factor binding sites (y-axis). Grey shading indicates overlap with allelic balanced ChIP-Seq peak while black indicates overlap with allelic imbalanced ChIP-Seq peak. Red X indicates overlapping SNP is tag SNP. Covariate along the top indicates the tissue while the covariate along the right indicates if the SNP is a literature reported risk SNP and what somatic driver it was associated with. FIG. 5E shows that rs11203152 located within regulatory dense region. Tracks show chromatin looping anchored by RNA Polymerase II (RNAPII), RAD21, AR or ERG in RWPE-1, LNCaP, VCaP or DU145 cell lines. FIG. 5F shows that the enriched number of chromatin loops was more than expected by chance in LNCaP and VCaP cell lines. Barplots shows number of anchors within 1 Mbp of rs11203152. Covariate along the bottom indicates cell line and target while the background shading indicates of the enrichment was more than expected by chance (FDR<0.05). The red X indicates the expected number of chromatin loop anchors based on 100,000 randomly sampled, equally sized regions.

FIGS. 6A-6D show results from studies described in Example 1, demonstrating that dQTL discovery p-value distribution is significantly skewed. FIG. 6A shows that dQTL discovery p-value distributions are significantly skewed towards smaller p-values. The p-value skew for each dQTL discovery for the top five most recurrent somatic drivers was compared to an empirically generated null distribution (iterations=1,000) and a p-value calculated as the number of null iterations with skew>real skew. Barplot shows the p-value from this permutation analysis. Horizontal line indicates P=0.05 and colours represent the dQTL discovery approach. FIG. 6B shows null skew distribution for T2E dQTL discovery from 1,000 iterations. Vertical lines represent real skew values for each dQTL approach. P-values along the top represent the number of null iterations with skew>real skew divided by the number of null iterations. FIG. 6C shows null skew distribution of clonal loss of ZNF292. FIG. 6D shows that dPRS accuracy predicts T2E (area under curve (AUC)=0.71 with 95% confidence interval). Receiver operating characteristic curve shows predictions from leave-one-out cross validation.

FIGS. 7A-7J show results from studies described in Example 1, demonstrating cohort characterization and risk dQTL replication. FIG. 7A shows clustering using identity-by-state as the distance metric showed no evidence of population substructure. Heatmap shows the identity-by-state values for all pairwise comparisons. The first covariate along the right shows the cluster provided by plink (v1.9). The second covariate indicates the original cohort the patient was published in. FIG. 7B shows landscape of somatic drivers in the discovery cohort. Somatic drivers are categorized as losses (blue), gains (red), SVs (purple), non-coding SNVs (pink) or coding SNVs (khaki). Barplot on the right shows the frequency of each driver in the discovery cohort. Covariate on the bottom indicates clinical characteristics of each patient including clinical ISUP grade group (ISUP), pre-treatment prostate serum antigen (PSA), clinical T category (cT) and age. Barplot on the top indicates the polygenic risk score (PRS), scaled between 0-1, for each patient. FIGS. 7C-7D show contingency tables of rs16901979 (FIG. 7C) and rs1859962 (FIG. 7D) associated with T2E in discovery cohort. FIGS. 7E-7F show Kaplan-Meier plots of rs1856888 (FIG. 7E) and rs1047303 (FIG. 7F) associated with metastasis-free survival. P-value from log-rank test. FIGS. 7G and 7H show contingency tables of rs1856888 (FIG. 7G) and rs1047303 (FIG. 7H) associated with clinical T category. P-values from Fisher's exact test. FIG. 7I shows a Kaplan-Meier plot of APOE genotypes associated with metastasis-free survival. P-value from log-rank test. FIG. 7J shows a contingency table of APOE2 and APOE4 associated with GR count. OR and p-value from Fisher's exact test.

FIGS. 8A-8J show results from studies described in Example 1, demonstrating that genetic risk inversely associated with somatic mutation burden. FIGS. 8A-8B show that PRS is negatively correlated with PGA in both discovery (FIG. 8A) and replication (FIG. 8B) cohort. FIGS. 8C-8D show that association between PGA and PRS was stronger when only considering subclonal CNAs (FIG. 8C) than clonal CNAs (FIG. 8D). Key indicates fold change (FC) and p-value between >75% and <25% groups from Mann-Whitney test. Boxplot represents median, 0.25 and 0.75 quantiles with whiskers at 1.5× interquartile range. FIGS. 8E-8G shows that PRS was not consistently associated with SNV mutation rate (SNV/Mbp) in the discovery (FIG. 8E) and replication (FIG. 8F) cohorts or GR count (FIG. 8G). FIG. 8H shows coefficients from linear model quantifying association between PRS and PGA or number of driver mutations with or without adjustment of age of diagnosis. Error bars given 95% confidence interval and background shading reflects p-value<0.05. FIG. 8I shows a QQ plot of expected −log₁₀ p-values vs observed −log₁₀ p-values for association of individual PRS SNPs with 37 drivers. FIG. 8J shows a comparison of ORs in discovery cohort, x-axis and the replication cohort, y-axis, for 10 risk dQTLs. Horizontal and vertical dotted lines represent OR=1 and diagonal line represents y=x. P-values from logistic regression correcting for first two genetic principal components and somatic mutation burden in replication cohort.

FIGS. 9A-9I show results from studies described in Example 1, demonstrating FIG. 9A shows a sensitivity plot of number of discovered tag linear local dQTLs based on increasing distance from gene boundaries. FIG. 9B shows a barplot of number of variants tested per somatic driver based on linear definition of local dQTL. Covariate along the top indicates the type of somatic driver event. c) Comparison of ORs for linear local dQTLs with CNA drivers based on WGS profiling, x-axis and array profiling, y-axis. Horizontal and vertical dotted lines represent OR=1 and diagonal line represents y=x. Halo around points indicates FDR<0.1 in array-profiled cohort. FIGS. 9D-9F show comparison of ORs in discovery, x-axis, vs ovarian (FIG. 9D), pancreatic (FIG. 9E) or breast (FIG. 9F) cancer, y-axis. Only testing dQTLs involving somatic drivers with recurrence rate >5% in each cancer type. Horizontal and vertical dotted lines represent OR=1 and diagonal line represents y=x. Halo around points indicates FDR<0.1 in replication cohort. FIG. 9G shows contingency tables of rs11203152 associated with clonal loss of TMPRSS2 in ovarian cancer. FIGS. 9H-9I show contingency tables of rs76748266 with gain of NCOA2 in discovery (FIG. 9H) and pancreatic cancer (FIG. 9I) cohorts.

FIGS. 10A-10G show results from studies described in Example 1, demonstrating spatial local dQTLs discovery. FIG. 10A shows a barplot showing the number of variants tested per somatic driver based on spatial definition of local dQTL. Covariate along the top indicates the type of somatic driver. FIG. 10B shows a comparison of ORs for spatial local dQTLs with CNA drivers based on WGS profiling, x-axis and array profiling, y-axis. Horizontal and vertical dotted lines represent OR=1 and diagonal line represents y=x. Halo around points indicates FDR<0.1 in array-profiled cohort. FIGS. 10C-10E show a comparison of ORs in discovery, x-axis, vs ovarian (FIG. 10C), pancreatic (FIG. 10D) or breast (FIG. 10E) cancer, y-axis. Only testing dQTLs involving somatic drivers with recurrence rate >5% in each cancer type. Horizontal and vertical dotted lines represent OR=1 and diagonal line represents y=x. Halo around points indicates FDR<0.1 in replication cohort. FIGS. 10F-10G show contingency tables of rs12385878 (FIG. 10F) and rs7320595 (FIG. 10G) associated with clonal loss of RB1 in breast cancer.

FIGS. 11A-11I show results from studies described in Example 1, demonstrating enhancer local dQTLs discovery. FIG. 11A shows a barplot showing the number of variants tested per somatic driver based on enhancer definition of local dQTL. Covariate along the top indicates the type of somatic driver. FIG. 11B shows a comparison of ORs for enhancer local dQTLs with CNA drivers based on WGS profiling, x-axis and array profiling, y-axis. Horizontal and vertical dotted lines represent OR=1 and diagonal line represents y=x. FIGS. 11C-11E show comparison of ORs in discovery, x-axis, vs ovarian (FIG. 11C), pancreatic (FIG. 11D) or breast (FIG. 11E) cancer, y-axis. Only testing dQTLs involving somatic drivers with recurrence rate >5% in each cancer type. Horizontal and vertical dotted lines represent OR=1 and diagonal line represents y=x. FIGS. 11F-11G show contingency tables of rs796498559 associated with loss of FBXO31 in discovery (FIG. 11F) and pancreatic cancer (FIG. 11G) cohorts. FIG. 11H shows candidate dQTL analysis considering 43 discovery dQTL SNPs. Dot size and colour represents OR magnitude and direction. Background shading indicates FDR. Covariate along the top represents the somatic driver type while the covariate along the right indicates the original somatic driver discovered for that SNP. FIG. 11I shows comparison of ORs in discovery, x-axis, vs replication, y-axis, cohort of 18 distal dQTLs. Horizontal and vertical dotted lines represent OR=1 and diagonal line represents y=x.

FIGS. 12A-12T show results from studies described in Example 1, demonstrating molecular characterization of dQTLs. FIGS. 12A-12B show comparison of ORs for a subset of 16 dQTLs in the discovery FIG. 12A or replication FIG. 12B cohorts vs EOPC cohort. Horizontal and vertical dotted lines represent OR=1 and diagonal line represents y=x. Halo around points indicates FDR<0.1 in EOPC cohort. FIG. 12C shows a schematic of characterization of dQTLs. FIG. 12D shows overlap of dQTLs, x-axis, with histone modifications and AR binding in primary patient samples, y-axis. Shading indicates the number of patients that show overlap. Covariate along the top indicates the somatic driver each SNP is associated with. FIG. 12E shows dQTLs overlap ChIP-Seq peaks in LNCaP, PC3, 22Rv1, VCaP and RWPE1 cell lines, y-axis. Shading indicates number of dQTLs overlapping each target and treatment pair. FIG. 12F shows volcano plot of candidate eQTL results testing each dQTL SNP for association with mRNA abundance of its associated driver gene. Y-axis gives −log₁₀ FDR and x-axis gives β from linear regression model. Horizontal line indicates FDR=0.05 and red points indicate nominally significant eQTLs (P<0.05). FIGS. 12G-12I show that five dQTLs were nominally significant eQTLs (P<0.05). Boxplot shows mRNA abundance (purple) for gene in title stratified by the genotype, x-axis, of the SNP indicated in the title. Statistics from linear regression model and the number of samples with each genotype is indicated in parenthesis next to the genotypes along the x-axis. Boxplot represents median, 0.25 and 0.75 quantiles with whiskers at 1.5× interquartile range. Only one plot presented for SNPs in strong LD. FIG. 12J shows that one nominally significant eQTL was also a pQTL: rs7320595 associated with RB1 protein abundance. Red points indicate protein abundance. FIG. 12K shows a volcano plot of local eQTL results. FIG. 12L shows a volcano plot of pQTL results. Significant pQTLs (FDR<0.05) labeled. FIGS. 12M-12R show that three nominally significant eQTLs were also significant pQTLs. FIG. 12S shows a barplot indicating a number of somatic SNVs, y-axis, within ±10 kbp around each dQTL, x-axis. Covariate along the top indicates the somatic driver each dQTL is associated with. FIG. 12T shows a summary of molecular and clinical characterization of dQTLs. Grey indicates dQTL was association with methylation (meQTL), RNA abundance (eQTL), protein abundance (pQTL), transcription factor binding, histone modification, ISUP grade group, biochemical recurrence (BCR) or risk of prostate cancer diagnosis (PCa Risk); middle heatmap shows if dQTL replicated in meta-analysis or the replication cohort; covariate on the left illustrates the somatic driver the dQTL is associated with.

FIGS. 13A-130 show results from studies described in Example 1, demonstrating clinical characterization of dQTLs. FIGS. 13A-13C show comparison of allelic frequencies for 43 dQTLs in European, x-axis vs East Asian, y-axis, populations (FIG. 13A), European vs. African populations (FIG. 13B) or within European populations (FIG. 13C). Halo indicates SNP has significantly different allele frequencies in two populations. FIG. 13D shows a contingency table of rs11203152 associated with loss of TMPRSS2 in 115 African men. FIG. 13E shows a contingency table of rs848048 associated with SNVs in FOXA1 UTR in 115 African men. FIG. 13F shows a forest plot showing Hazard Ratios, x-axis, from survival analysis of dQTLs, y-axis, with biochemical recurrence. Error bars represent 95% confidence intervals. Vertical dotted line represents HR=1. Background shading indicates P<0.05 and covariate on the right indicates the somatic driver event the SNP is associated with. FIGS. 13G-113J show Kaplan-Meier plots for the four dQTLs with P<0.05: rs2837396 (FIG. 13G), rs2839469 (FIG. 13H), rs5759167 (FIG. 13I) and rs12824766 (FIG. 13J). Statistics on KM plots have been adjusted for primary treatment. FIGS. 13K-13N show contingency tables of association between rs439864 (FIG. 13K), rs374296 (FIG. 13L), rs13279615 (FIG. 13M) and rs388012 (FIG. 13N) and ISUP grade group. FDR from ordinal linear regression. FIG. 13O shows a barplot showing effect size and p-value from prostate cancer GWAS⁹ for 32 non-risk dQTLs, x-axis, with summary statistics from GWAS. Horizontal line indicates P=0.05. Covariate along the top indicates associated somatic driver.

FIGS. 14A-14I show results from studies described in Example 1, demonstrating enrichment of sub-significance threshold dQTLs. FIGS. 14A-14C show heatmaps displaying estimated power considering increasing ORs (x-axis) and allele frequencies (y-axis) for somatic driver recurrence=0.50 (FIG. 14A), 0.20 (FIG. 14B) and 0.05 (FIG. 14C). Shading indicates estimated power with yellow=0 and purple=1. Grey indicates power could not be calculated. FIG. 14D shows null skew distribution for clonal loss of RB1 dQTL discovery from 1,000 iterations. Vertical lines represent real skew values for each dQTL approach. P-values along the top represent the number of null iterations with skew >real skew divided by the number of null iterations. FIG. 14E shows null skew distribution of clonal loss of NKX3-1. FIG. 14F shows null skew distribution of clonal loss of TMPRSS2. FIGS. 14G-14H show Q-Q plots of T2E linear local dQTLs (FIG. 14G) and clonal loss of ZNF292 spatial local dQTLs (FIG. 14H). FIG. 14I shows a heatmap showing R for SNPs, x-axis, included in at least 50% of leave-one-out cross validation dPRS. Each column represents a dPRS built on all by one sample. Grey indicates SNP was not included in that score. Barplot on the right indicates the number of dPRS SNP was included in.

### DETAILED DESCRIPTION OF THE INVENTION

The present disclosure is based, at least in part, on the discovery and characterization of individual, germline single nucleotide polymorphisms (SNPs), described herein at driver quantitative trait loci (dQTL), which influence acquisition of specific cancer driver mutations, including for example TMPRSS2-ERG fusion and FOXA1 point mutations. As disclosed herein, dQTLs may be used in the prediction of molecular features (e.g., genetic abnormalities) of a cancer before diagnosis, as well as for informing diagnosis, characterization, and treatment of cancer.

### I. Prediction and Characterization of Cancer Genetic Abnormalities

Aspects of the present disclosure are directed to prediction and/or characterization of certain cancer genetic abnormalities based on genotypic analysis of a subject. In some embodiments, disclosed herein are methods for predicting the development of a cancer having one or more particular genetic abnormalities. As disclosed herein, a “genetic abnormality” of a cancer describes any genetic characteristic (e.g., genetic sequence, gene expression, epigenetic feature, etc.) which is present in a cancer cell from a subject and is not present in a germline cell from the subject. Examples of genetic abnormalities contemplated herein include chromosomal translocations, base substitutions, insertions, deletions, gene fusions, and various types of genetic mutations, including nonsense mutations, missense mutations, point mutations, and frameshift mutations. Also contemplated are epigenetic abnormalities, including increased or decreased methylation of one or more regions of cancer DNA. In some embodiments, genetic abnormalities of the disclosure are cancer driver mutations. In some embodiments, a genetic abnormality of the disclosure is a simple somatic mutation (also “SSM”, i.e., a single nucleotide variant, insertion, or deletion). In some embodiments, a genetic abnormality of the disclosure is a structural variant (including, e.g., copy number variations (CNVs), inversions, insertions, deletions and other complex rearrangements).

In some embodiments, disclosed are methods for prediction of a cancer genetic abnormality based on genotypic analysis of a subject. For example, certain germline polymorphisms are described herein as associated with particular cancer genetic abnormalities. Thus, embodiments of the disclosure include genotying a subject without cancer as having a particular polymorphism and predicting that any cancer developed in the subject will have, or has an increased likelihood of having, the associated genetic abnormality. In one specific example, disclosed is a method for predicting that any prostate cancer developed in a subject will have, or will have an increased likelihood of having, a TMPRSS2-ERG fusion comprising detecting the presence of one or more SNPs selected from rs111620024, rs12500426, rs7679673, rs12653946, rs2837396, and rs2839469 in the subject. As used herein, a “TMPRSS2-ERG fusion” describes any nucleic acid or protein comprising sequence from both TMPRSS2 and ERG. In some embodiments, a TMPRSS2-ERG fusion described a nucleic acid having nucleotide sequence from both the TMPRSS2 gene and the ERG gene. In some embodiments, a TMPRSS2-ERG fusion described a protein having polypeptide sequence from both the TMPRSS2 protein and the ERG protein. Various examples of a TMPRSS2-ERG fusion are described in, for example, Nam R K, Cancer Biol Ther. 2007 January; 6(1):40-5; Tomlins S A, Science. 2005 Oct. 28; 310(5748):644-8; and Hu Y, Clin Cancer Res. 2008 Aug. 1; 14(15):4719-25, incorporated herein by reference in their entirety. In some embodiments, a TMPRSS2-ERG fusion is a nucleic acid or protein as characterized by GenBank accession no. EU432099. Additional genetic abnormalities and associated polymorphisms are described further herein.

In some embodiments, disclosed are methods for characterization of a cancer genetic abnormality based on genotypic analysis of a subject. For example, certain germline polymorphisms are described herein as associated with particular cancer genetic abnormalities. Thus, embodiments of the disclosure include genotying a subject with cancer as having a particular polymorphism and characterizing the cancer as having, or having an increased likelihood of having, the associated genetic abnormality. In one specific example, disclosed is a method for characterizing a prostate cancer of a subject as having, or having an increased likelihood of having, a TMPRSS2-ERG fusion comprising detecting the presence of one or more SNPs selected from rs111620024, rs12500426, rs7679673, rs12653946, rs2837396, and rs2839469 in the subject.

Certain example polymorphisms and associations of the present disclosure are provided in Table 1.

TABLE 1
Example SNPs and associated genetic abnormalities in prostate cancer and other cancers

| SNP (rs | SNP (chromosomal location - | | |
|---|---|---|---|
| identifier) | GRch38.p12) | Gene | Genetic Abnormality |
| rs28629385 | chr8: 127538177 | MYC | Increased copy number |
| | G > A, C | | |
| rs55667421 | chr8: 127535470 T > A | MYC | Increased copy number |
| rs76748266 | chr8: 69765596 C > T | MYC | Increased copy number |
| rs304605 | chr8: 70027864 T > A, C, G | NCOA2 | Increased copy number |
| rs422651 | chr8: 70016921 G > A, T | NCOA2 | Increased copy number |
| rs439864 | chr8: 70006603 T > A, C | NCOA2 | Increased copy number |
| rs6472505 | chr8: 69994823 C > A | NCOA2 | Increased copy number |
| rs28629385 | chr8: 127538177 G > A, C | NCOA2 | Increased copy number |
| rs55667421 | chr8: 127535470 T > A | NCOA2 | Increased copy number |
| rs76748266 | chr8: 69765596 C > T | NCOA2 | Increased copy number |
| rs7679673 | chr4: 105140377 C > A, T | ARID4B | Reduced expression and/or deletion |
| rs12817741 | chr12: 12602365 G > A | CDKN1B | Reduced expression and/or deletion |
| rs12824766 | chr12: 12110057 T > C, G | CDKN1B | Reduced expression and/or deletion |
| rs141393446 | chr6: 87759407 A > G | CDKN1B | Reduced expression and/or deletion |
| rs141853059 | chr12: 12618666 A > G | CDKN1B | Reduced expression and/or deletion |
| rs57526507 | chr6: 87740727 G > A | CDKN1B | Reduced expression and/or deletion |
| rs61915608 | chr12: 12656519 C > T | CDKN1B | Reduced expression and/or deletion |
| rs111620024 | chr5: 96662687 C > T | CHD1 | Reduced expression and/or deletion |
| rs12933820 | chr16: 87911666 T > C | FBXO31 | Reduced expression and/or deletion |
| rs4441280 | chr16: 87914367 A > G | FBXO31 | Reduced expression and/or deletion |
| rs796498559 | chr16: 87913665 T > A, C, G | FBXO31 | Reduced expression and/or deletion |
| rs8046279 | chr 16: 87913400 G > A, C, T | FBXO31 | Reduced expression and/or deletion |
| rs11480453 | chr20: 32759707 AA > —, A, AAA, AAAA, | MAST4 | Reduced expression and/or deletion |
| | AAAAA, AAAAAA | | |
| rs11650494 | chr17: 49267824 G > A | MAST4 | Reduced expression and/or deletion |
| rs141393446 | chr6: 87759407 A > G | MAST4 | Reduced expression and/or deletion |
| rs57526507 | chr6: 87740727 G > A | MAST4 | Reduced expression and/or deletion |
| rs304605 | chr8: 70027864 T > A, C, G | MCM6 | Reduced expression and/or deletion |
| rs422651 | chr8: 70016921 G > A, T | MCM6 | Reduced expression and/or deletion |
| rs439864 | chr8: 70006603 T > A, C | MCM6 | Reduced expression and/or deletion |
| rs80130819 | chr12: 48025835 A > C, G | MCM6 | Reduced expression and/or deletion |
| rs4676882 | chr3: 72611428 T > C | PPP4R2 | Reduced expression and/or deletion |
| rs73111031 | chr3: 71978157 C > T | PPP4R2 | Reduced expression and/or deletion |
| | chr12: 131939920 G > T | RAN | Reduced expression and/or deletion |
| rs1696428 | chr12: 130042181 A > G | RAN | Reduced expression and/or deletion |
| rs12385878 | chr13: 49282062 C > A, T | RB1 | Reduced expression and/or deletion |
| rs1928107 | chr13: 49001371 T > A, C, G | RB1 | Reduced expression and/or deletion |
| rs7320595 | chr13: 49281140 C > G, T | RB1 | Reduced expression and/or deletion |
| rs11203152 | chr21: 41599278 G > A | TMPRSS2 | Reduced expression and/or deletion |
| rs12500426 | chr4: 94593458 A > C | TMPRSS2 | Reduced expression and/or deletion |
| rs12653946 | chr5: 1895715 C > A, T | TMPRSS2 | Reduced expression and/or deletion |
| rs13048402 | chr21: 41593157 C > A, G, T | TMPRSS2 | Reduced expression and/or deletion |
| rs2837396 | chr21: 40053334 G > A | TMPRSS2 | Reduced expression and/or deletion |
| rs5759167 | chr22: 43104206 G > A, T | TMPRSS2 | Reduced expression and/or deletion |
| rs3771570 | chr2: 241443449 C > T | TP63 | Reduced expression and/or deletion |
| rs12817741 | chr12: 12602365 G > A | ZNF292 | Reduced expression and/or deletion |
| rs141393446 | chr6: 87759407 A > G | ZNF292 | Reduced expression and/or deletion |
| rs2837396 | chr21: 40053334 G > A | ZNF292 | Reduced expression and/or deletion |
| rs57526507 | chr6: 87740727 G > A | ZNF292 | Reduced expression and/or deletion |
| rs61915608 | chr12: 12656519 C > T | ZNF292 | Reduced expression and/or deletion |
| rs77404504 | chr14: 38243421 G > C, T | FOXA1 | Simple somatic mutation (single nucleotide |
| | | | variant or indel) in UTR |
| rs848047 | chr14: 36858645 T > C | FOXA1 | Simple somatic mutation (single nucleotide |
| | | | variant or indel) in UTR |
| rs848048 | chr14: 36858578 T > A, C | FOXA1 | Simple somatic mutation (single nucleotide |
| | | | variant or indel) in UTR |
| rs4924487 | chr15: 40630717 G > C, T | PCM1 | Structural variant |
| rs5919432 | chrX: 67801708 C > A, G, T | PCM1 | Structural variant |
| rs12824766 | chr12: 12110057 T > C, G | TTC28 | Structural variant |
| rs5763061 | chr22: 29219743 C > T | TTC28 | Structural variant |
| rs111620024 | chr5: 96662687 C > T | TMPRSS2 | TMPRSS2-ERG fusion |
| rs12500426 | chr4: 94593458 A > C | TMPRSS2 | TMPRSS2-ERG fusion |
| rs12653946 | chr5: 1895715 C > A, T | TMPRSS2 | TMPRSS2-ERG fusion |
| rs2837396 | chr21: 40053334 G > A | TMPRSS2 | TMPRSS2-ERG fusion |
| rs2839469 | chr21: 42120315 G > A, C | TMPRSS2 | TMPRSS2-ERG fusion |
| rs7679673 | chr4: 105140377 C > A, T | TMPRSS2 | TMPRSS2-ERG fusion |

### II. Therapeutic Methods

Aspects of the present disclosure comprise therapeutic methods and compositions for use thereof. Compositions of the disclosure may be used for in vivo, in vitro, and/or ex vivo administration.

### A. Cancer Therapy

In some embodiments, the disclosed methods comprise administering a cancer therapy to a subject or patient. The cancer therapy may be chosen based on an expression level measurements, alone or in combination with the clinical risk score calculated for the subject. The cancer therapy may be chosen based on a genotype of a subject. The cancer therapy may be chosen based on the presence or absence of one or more polymorphisms in a subject. In some embodiments, the cancer therapy comprises a local cancer therapy. In some embodiments, the cancer therapy excludes a systemic cancer therapy. In some embodiments, the cancer therapy excludes a local therapy. In some embodiments, the cancer therapy comprises a local cancer therapy without the administration of a system cancer therapy. In some embodiments, the cancer therapy comprises an immunotherapy, which may be a checkpoint inhibitor therapy. Any of these cancer therapies may also be excluded. Combinations of these therapies may also be administered.

The term “cancer,” as used herein, may be used to describe a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus. In some embodiments, the cancer is a Stage I cancer. In some embodiments, the cancer is a Stage II cancer. In some embodiments, the cancer is a Stage III cancer. In some embodiments, the cancer is a Stage IV cancer.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, disclosed are methods for treating cancer originating from the prostate. In some embodiments, the cancer is prostate cancer. In some embodiments, the cancer is breast cancer. In some embodiments, the cancer is a recurrent cancer. In some embodiments, the cancer is an immunotherapy-resistant cancer.

Methods may involve the determination, administration, or selection of an appropriate cancer “management regimen” and predicting the outcome of the same. As used herein the phrase “management regimen” refers to a management plan that specifies the type of examination, screening, diagnosis, surveillance, care, and treatment (such as dosage, schedule and/or duration of a treatment) provided to a subject in need thereof (e.g., a subject diagnosed with cancer).

Biomarkers, like SNPs (e.g., one or more SNPs of Tables 1), can, in some cases, predict the efficacy of certain therapeutic regimens and can be used to identify patients who will receive benefit from a particular therapy.

### B. Radiotherapy

In some embodiments, a radiotherapy, such as ionizing radiation, is administered to a subject. As used herein, “ionizing radiation” means radiation comprising particles or photons that have sufficient energy or can produce sufficient energy via nuclear interactions to produce ionization (gain or loss of electrons). A preferred non-limiting example of ionizing radiation is an x-radiation. Means for delivering x-radiation to a target tissue or cell are well known in the art.

In some embodiments, the radiotherapy can comprise external radiotherapy, internal radiotherapy, radioimmunotherapy, or intraoperative radiation therapy (IORT). In some embodiments, the external radiotherapy comprises three-dimensional conformal radiation therapy (3D-CRT), intensity modulated radiation therapy (IMRT), proton beam therapy, image-guided radiation therapy (IGRT), or stereotactic radiation therapy. In some embodiments, the internal radiotherapy comprises interstitial brachytherapy, intracavitary brachytherapy, or intraluminal radiation therapy. In some embodiments, the radiotherapy is administered to a primary tumor.

In some embodiments, the amount of ionizing radiation is greater than 20 Gy and is administered in one dose. In some embodiments, the amount of ionizing radiation is 18 Gy and is administered in three doses. In some embodiments, the amount of ionizing radiation is at least, at most, or exactly 0.5, 1, 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 Gy (or any derivable range therein). In some embodiments, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some embodiments, the amount of radiotherapy administered to a subject may be presented as a total dose of radiotherapy, which is then administered in fractionated doses. For example, in some embodiments, the total dose is 50 Gy administered in 10 fractionated doses of 5 Gy each. In some embodiments, the total dose is 50-90 Gy, administered in 20-60 fractionated doses of 2-3 Gy each. In some embodiments, the total dose of radiation is at least, at most, or about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 Gy (or any derivable range therein). In some embodiments, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein). In some embodiments, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some embodiments, at least, at most, or exactly 1, 2, 3, 4, 5, 6, 7, 8, 9, 10, 11, or 12 (or any derivable range therein) fractionated doses are administered per day. In some embodiments, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

### C. Cancer Immunotherapy

In some embodiments, the methods comprise administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Various immunotherapies are known in the art, and examples are described below.

### 1. Checkpoint Inhibitors and Combination Treatment

Embodiments of the disclosure may include administration of immune checkpoint inhibitors, examples of which are further described below. As disclosed herein, “checkpoint inhibitor therapy” (also “immune checkpoint blockade therapy”, “immune checkpoint therapy”, “ICT,” “checkpoint blockade immunotherapy,” or “CBI”), refers to cancer therapy comprising providing one or more immune checkpoint inhibitors to a subject suffering from or suspected of having cancer.

a. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 inhibitor is a molecule that inhibits the binding of PD-1 to its ligand binding partners. In a specific aspect, the PD-1 ligand binding partners are PDL1 and/or PDL2. In another embodiment, a PDL1 inhibitor is a molecule that inhibits the binding of PDL1 to its binding partners. In a specific aspect, PDL1 binding partners are PD-1 and/or B7-1. In another embodiment, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the V_{H} region of nivolumab, pembrolizumab, or pidilizumab, and the CDR1, CDR2 and CDR3 domains of the V_{L} region of nivolumab, pembrolizumab, or pidilizumab. In another embodiment, the antibody competes for binding with and/or binds to the same epitope on PD-1, PDL1, or PDL2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

b. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or V_{H} and/or V_{L} domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO 01/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the V_{H} region of tremelimumab or ipilimumab, and the CDR1, CDR2 and CDR3 domains of the V_{L} region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

c. LAG3

Another immune checkpoint that can be targeted in the methods provided herein is the lymphocyte-activation gene 3 (LAG3), also known as CD223 and lymphocyte activating 3. The complete mRNA sequence of human LAG3 has the Genbank accession number NM_002286. LAG3 is a member of the immunoglobulin superfamily that is found on the surface of activated T cells, natural killer cells, B cells, and plasmacytoid dendritic cells. LAG3's main ligand is MHC class II, and it negatively regulates cellular proliferation, activation, and homeostasis of T cells, in a similar fashion to CTLA-4 and PD-1, and has been reported to play a role in Treg suppressive function. LAG3 also helps maintain CD8+ T cells in a tolerogenic state and, working with PD-1, helps maintain CD8 exhaustion during chronic viral infection. LAG3 is also known to be involved in the maturation and activation of dendritic cells. Inhibitors of the disclosure may block one or more functions of LAG3 activity.

In some embodiments, the immune checkpoint inhibitor is an anti-LAG3 antibody (e.g., a human antibody, a humanized antibody, or a chimeric antibody), an antigen binding fragment thereof, an immunoadhesin, a fusion protein, or oligopeptide.

Anti-human-LAG3 antibodies (or V_{H} and/or V_{L} domains derived therefrom) suitable for use in the present methods can be generated using methods well known in the art. Alternatively, art recognized anti-LAG3 antibodies can be used. For example, the anti-LAG3 antibodies can include: GSK2837781, IMP321, FS-118, Sym022, TSR-033, MGD013, BI754111, AVA-017, or GSK2831781. The anti-LAG3 antibodies disclosed in: U.S. Pat. No. 9,505,839 (BMS-986016, also known as relatlimab); U.S. Pat. No. 10,711,060 (IMP-701, also known as LAG525); U.S. Pat. No. 9,244,059 (IMP731, also known as H5L7BW); U.S. Pat. No. 10,344,089 (25F7, also known as LAG3.1); WO 2016/028672 (MK-4280, also known as 28G-10); WO 2017/019894 (BAP050); Burova E., et al., J. ImmunoTherapy Cancer, 2016; 4(Supp. 1):P195 (REGN3767); Yu, X., et al., mAbs, 2019; 11:6 (LBL-007) can be used in the methods disclosed herein. These and other anti-LAG-3 antibodies useful in the claimed invention can be found in, for example: WO 2016/028672, WO 2017/106129, WO 2017062888, WO 2009/044273, WO 2018/069500, WO 2016/126858, WO 2014/179664, WO 2016/200782, WO 2015/200119, WO 2017/019846, WO 2017/198741, WO 2017/220555, WO 2017/220569, WO 2018/071500, WO 2017/015560; WO 2017/025498, WO 2017/087589, WO 2017/087901, WO 2018/083087, WO 2017/149143, WO 2017/219995, US 2017/0260271, WO 2017/086367, WO 2017/086419, WO 2018/034227, and WO 2014/140180. The teachings of each of the aforementioned publications are hereby incorporated by reference. Antibodies that compete with any of these art-recognized antibodies for binding to LAG3 also can be used.

In some embodiments, the inhibitor comprises the heavy and light chain CDRs or VRs of an anti-LAG3 antibody. Accordingly, in one embodiment, the inhibitor comprises the CDR1, CDR2, and CDR3 domains of the V_{H} region of an anti-LAG3 antibody, and the CDR1, CDR2 and CDR3 domains of the V_{L} region of an anti-LAG3 antibody. In another embodiment, the antibody has at least about 70, 75, 80, 85, 90, 95, 97, or 99% (or any derivable range therein) variable region amino acid sequence identity with the above-mentioned antibodies.

d. TIM-3

Another immune checkpoint that can be targeted in the methods provided herein is the T-cell immunoglobulin and mucin-domain containing-3 (TIM-3), also known as hepatitis A virus cellular receptor 2 (HAVCR2) and CD366. The complete mRNA sequence of human TIM-3 has the Genbank accession number NM_032782. TIM-3 is found on the surface IFNγ-producing CD4+Th1 and CD8+ Tcl cells. The extracellular region of TIM-3 consists of a membrane distal single variable immunoglobulin domain (IgV) and a glycosylated mucin domain of variable length located closer to the membrane. TIM-3 is an immune checkpoint and, together with other inhibitory receptors including PD-1 and LAG3, it mediates the T-cell exhaustion. TIM-3 has also been shown as a CD4+Th1-specific cell surface protein that regulates macrophage activation. Inhibitors of the disclosure may block one or more functions of TIM-3 activity.

In some embodiments, the immune checkpoint inhibitor is an anti-TIM-3 antibody (e.g., a human antibody, a humanized antibody, or a chimeric antibody), an antigen binding fragment thereof, an immunoadhesin, a fusion protein, or oligopeptide.

Anti-human-TIM-3 antibodies (or V_{H} and/or V_{L} domains derived therefrom) suitable for use in the present methods can be generated using methods well known in the art. Alternatively, art recognized anti-TIM-3 antibodies can be used. For example, anti-TIM-3 antibodies including: MBG453, TSR-022 (also known as Cobolimab), and LY3321367 can be used in the methods disclosed herein. These and other anti-TIM-3 antibodies useful in the claimed invention can be found in, for example: U.S. Pat. Nos. 9,605,070, 8,841,418, US2015/0218274, and US 2016/0200815. The teachings of each of the aforementioned publications are hereby incorporated by reference. Antibodies that compete with any of these art-recognized antibodies for binding to TIM-3 also can be used.

In some embodiments, the inhibitor comprises the heavy and light chain CDRs or VRs of an anti-TIM-3 antibody. Accordingly, in one embodiment, the inhibitor comprises the CDR1, CDR2, and CDR3 domains of the V_{H} region of an anti-TIM-3 antibody, and the CDR1, CDR2 and CDR3 domains of the V_{L} region of an anti-TIM-3 antibody. In another embodiment, the antibody has at least about 70, 75, 80, 85, 90, 95, 97, or 99% (or any derivable range or value therein) variable region amino acid sequence identity with the above-mentioned antibodies.

### 2. Activator of Co-Stimulatory Molecules

In some embodiments, the immunotherapy comprises an activator (also “agonist”) of a co-stimulatory molecule. In some embodiments, the agonist comprises an agonist of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Agonists include activating antibodies, polypeptides, compounds, and nucleic acids.

### 3. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

### 4. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Example CAR-T therapies include Tisagenlecleucel (Kymriah) and Axicabtagene ciloleucel (Yescarta).

### 5. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an example interleukin cytokine therapy.

### 6. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

It is contemplated that a cancer treatment may exclude any of the cancer treatments described herein. Furthermore, embodiments of the disclosure include patients that have been previously treated for a therapy described herein, are currently being treated for a therapy described herein, or have not been treated for a therapy described herein. In some embodiments, the patient is one that has been determined to be resistant to a therapy described herein. In some embodiments, the patient is one that has been determined to be sensitive to a therapy described herein. For example, the patient may be one that has been determined to be sensitive to an immune checkpoint inhibitor therapy based on a determination that the patient has or previously had pancreatitis.

### D. Oncolytic Virus

In some embodiments, the cancer therapy comprises an oncolytic virus. An oncolytic virus is a virus that preferentially infects and kills cancer cells. As the infected cancer cells are destroyed by oncolysis, they release new infectious virus particles or virions to help destroy the remaining tumor. Oncolytic viruses are thought not only to cause direct destruction of the tumor cells, but also to stimulate host anti-tumor immune responses for long-term immunotherapy

### E. Chemotherapies

In some embodiments, a therapy of the present disclosure comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some embodiments, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m2 to about 20 mg/m2 for 5 days every three weeks for a total of three courses being contemplated in certain embodiments.

Other suitable chemotherapeutic agents include antimicrotubule agents, e.g., Paclitaxel (“Taxol”) and doxorubicin hydrochloride (“doxorubicin”). Doxorubicin is absorbed poorly and is preferably administered intravenously. In certain embodiments, appropriate intravenous doses for an adult include about 60 mg/m² to about 75 mg/m² at about 21-day intervals or about 25 mg/m² to about 30 mg/m² on each of 2 or 3 successive days repeated at about 3 week to about 4 week intervals or about 20 mg/m² once a week.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

The amount of the chemotherapeutic agent delivered to a patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### F. Hormone Therapy

In some embodiments, a cancer therapy of the present disclosure is a hormone therapy. In particular aspects, a prostate cancer therapy is a prostate cancer hormone therapy. Various prostate cancer hormone therapies are known in the art and include, for example, luteinizing hormone-releasing hormone (LHRH) analogs, LHRH antagonists, androgen receptor antagonists, and androgen synthesis inhibitors.

### G. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### III. Sample Preparation

In certain aspects, methods involve obtaining a sample (also “biological sample”) from a subject. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. In certain embodiments the sample is obtained from a biopsy from esophageal tissue by any of the biopsy methods previously mentioned. In other embodiments the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, serum, plasma, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. Alternatively, the biological sample may be a cell-free sample, for example serum or plasma. The biological sample may be obtained using any method known to the art that can provide a sample suitable for the analytical methods described herein. The sample may be obtained by non-invasive methods including but not limited to: scraping of the skin or cervix, swabbing of the cheek, blood collection, saliva collection, urine collection, feces collection, or collection of menses, tears, or semen. In some embodiments, a sample comprises nucleic acids from the subject. In some embodiments, a sample comprises nucleic acids from one or more cancer cells from a subject. In some embodiments, a sample comprises tumor DNA (i.e., DNA from one or more cancer cells). In some embodiments, a sample comprises tumor RNA (i.e., RNA from one or more cancer cells). In some embodiments, a sample is a cell free sample. In some embodiments, a sample comprises cell free DNA (cfDNA). In some embodiments, the sample is a blood sample. In some embodiments, the sample is a saliva sample. In some embodiments, the sample is a urine sample.

The sample may be obtained by methods known in the art. In certain embodiments the samples are obtained by biopsy. In other embodiments the sample is obtained by swabbing, endoscopy, scraping, phlebotomy, or any other methods known in the art. In some cases, the sample may be obtained, stored, or transported using components of a kit of the present methods. In some cases, multiple samples, such as multiple esophageal samples may be obtained for diagnosis by the methods described herein. In other cases, multiple samples, such as one or more samples from one tissue type (for example esophagus) and one or more samples from another specimen (for example serum) may be obtained for diagnosis by the methods. In some cases, multiple samples such as one or more samples from one tissue type (e.g. esophagus) and one or more samples from another specimen (e.g. serum) may be obtained at the same or different times. Samples may be obtained at different times are stored and/or analyzed by different methods. For example, a sample may be obtained and analyzed by routine staining methods or any other cytological analysis methods.

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one embodiment, the sample is a fine needle aspirate of a esophageal or a suspected esophageal tumor or neoplasm. In some cases, the fine needle aspirate sampling procedure may be guided by the use of an ultrasound, X-ray, or other imaging device.

In some embodiments of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some embodiments of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some embodiments, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### IV. Assay Methods

a. Detection of Methylated DNA

Aspects of the methods include assaying nucleic acids to determine expression levels and/or methylation levels of nucleic acids. Assays for the detection of methylated DNA are known in the art. Example methods are described herein.

### 1. HPLC-UV

The technique of HPLC-UV (high performance liquid chromatography-ultraviolet), developed by Kuo and colleagues in 1980 (described further in Kuo K. C. et al., Nucleic Acids Res. 1980; 8:4763-4776, which is herein incorporated by reference) can be used to quantify the amount of deoxycytidine (dC) and methylated cytosines (5 mC) present in a hydrolysed DNA sample. The method includes hydrolyzing the DNA into its constituent nucleoside bases, the 5 mC and dC bases are separated chromatographically and, then, the fractions are measured. Then, the 5 mC/dC ratio can be calculated for each sample, and this can be compared between the experimental and control samples.

### 2. LC-MS/MS

Liquid chromatography coupled with tandem mass spectrometry (LC-MS/MS) is an high-sensitivity approach to HPLC-UV, which requires much smaller quantities of the hydrolysed DNA sample. In the case of mammalian DNA, of which ˜2%-5% of all cytosine residues are methylated, LC-MS/MS has been validated for detecting levels of methylation levels ranging from 0.05%-10%, and it can confidently detect differences between samples as small as ˜0.25% of the total cytosine residues, which corresponds to ˜5% differences in global DNA methylation. The procedure routinely requires 50-100 ng of DNA sample, although much smaller amounts (as low as 5 ng) have been successfully profiled. Another major benefit of this method is that it is not adversely affected by poor-quality DNA (e.g., DNA derived from FFPE samples).

### 3. ELISA-Based Methods

There are several commercially available kits, all enzyme-linked immunosorbent assay (ELISA) based, that enable the quick assessment of DNA methylation status. These assays include Global DNA Methylation ELISA, available from Cell Biolabs; Imprint Methylated DNA Quantification kit (sandwich ELISA), available from Sigma-Aldrich; EpiSeeker methylated DNA Quantification Kit, available from abcam; Global DNA Methylation Assay LINE—1, available from Active Motif; 5-mC DNA ELISA Kit, available from Zymo Research; MethylFlash Methylated DNA5-mC Quantification Kit and MethylFlash Methylated DNA5-mC Quantification Kit, available from Epigentek.

Briefly, the DNA sample is captured on an ELISA plate, and the methylated cytosines are detected through sequential incubations steps with: (1) a primary antibody raised against 5 Mc; (2) a labelled secondary antibody; and then (3) colorimetric/fluorometric detection reagents.

The Global DNA Methylation Assay—LINE-1 specifically determines the methylation levels of LINE-1 (long interspersed nuclear elements-1) retrotransposons, of which ˜17% of the human genome is composed. These are well established as a surrogate for global DNA methylation. Briefly, fragmented DNA is hybridized to biotinylated LINE-1 probes, which are then subsequently immobilized to a streptavidin-coated plate. Following washing and blocking steps, methylated cytosines are quantified using an anti-5 mC antibody, HRP-conjugated secondary antibody and chemiluminescent detection reagents. Samples are quantified against a standard curve generated from standards with known LINE-1 methylation levels. The manufacturers claim the assay can detect DNA methylation levels as low as 0.5%. Thus, by analysing a fraction of the genome, it is possible to achieve better accuracy in quantification.

### 4. LINE-1 Pyrosequencing

Levels of LINE-1 methylation can alternatively be assessed by another method that involves the bisulfite conversion of DNA, followed by the PCR amplification of LINE-1 conservative sequences. The methylation status of the amplified fragments is then quantified by pyrosequencing, which is able to resolve differences between DNA samples as small as ˜5%. Even though the technique assesses LINE-1 elements and therefore relatively few CpG sites, this has been shown to reflect global DNA methylation changes very well. The method is particularly well suited for high throughput analysis of cancer samples, where hypomethylation is very often associated with poor prognosis. This method is particularly suitable for human DNA, but there are also versions adapted to rat and mouse genomes.

### 5. AFLP and RFLP

Detection of fragments that are differentially methylated could be achieved by traditional PCR-based amplification fragment length polymorphism (AFLP), restriction fragment length polymorphism (RFLP) or protocols that employ a combination of both.

### 6. LUMA

The LUMA (luminometric methylation assay) technique utilizes a combination of two DNA restriction digest reactions performed in parallel and subsequent pyrosequencing reactions to fill-in the protruding ends of the digested DNA strands. One digestion reaction is performed with the CpG methylation-sensitive enzyme HpaII; while the parallel reaction uses the methylation-insensitive enzyme MspI, which will cut at all CCGG sites. The enzyme EcoRI is included in both reactions as an internal control. Both MspI and HpaII generate 5′-CG overhangs after DNA cleavage, whereas EcoRI produces 5′-AATT overhangs, which are then filled in with the subsequent pyrosequencing-based extension assay. Essentially, the measured light signal calculated as the HpaII/MspI ratio is proportional to the amount of unmethylated DNA present in the sample. As the sequence of nucleotides that are added in pyrosequencing reaction is known, the specificity of the method is very high and the variability is low, which is essential for the detection of small changes in global methylation. LUMA requires only a relatively small amount of DNA (250-500 ng), demonstrates little variability and has the benefit of an internal control to account for variability in the amount of DNA input.

### 7. Bisulfite Sequencing

The bisulfite treatment of DNA mediates the deamination of cytosine into uracil, and these converted residues will be read as thymine, as determined by PCR-amplification and subsequent Sanger sequencing analysis. However, 5 mC residues are resistant to this conversion and, so, will remain read as cytosine. Thus, comparing the Sanger sequencing read from an untreated DNA sample to the same sample following bisulfite treatment enables the detection of the methylated cytosines. With the advent of next-generation sequencing (NGS) technology, this approach can be extended to DNA methylation analysis across an entire genome. To ensure complete conversion of non-methylated cytosines, controls may be incorporated for bisulfite reactions.

Whole genome bisulfite sequencing (WGBS) is similar to whole genome sequencing, except for the additional step of bisulfite conversion. Sequencing of the 5 mC-enriched fraction of the genome is not only a less expensive approach, but it also allows one to increase the sequencing coverage and, therefore, precision in revealing differentially-methylated regions. Sequencing could be done using any existing NGS platform; Illumina and Life Technologies both offer kits for such analysis.

Bisulfite sequencing methods include reduced representation bisulfite sequencing (RRBS), where only a fraction of the genome is sequenced. In RRBS, enrichment of CpG-rich regions is achieved by isolation of short fragments after MspI digestion that recognizes CCGG sites (and it cut both methylated and unmethylated sites). It ensures isolation of ˜85% of CpG islands in the human genome. Then, the same bisulfite conversion and library preparation is performed as for WGBS. The RRBS procedure normally requires ˜100 ng-1 μg of DNA.

8. Methods that Exclude Bisulfite Conversion

In some aspects, direct detection of modified bases without bisulfite conversion may be used to detect methylation. Pacific Biosciences company has developed a way to detect methylated bases directly by monitoring the kinetics of polymerase during single molecule sequencing and offers a commercial product for such sequencing (further described in Flusberg B. A., et al., Nat. Methods. 2010; 7:461-465, which is herein incorporated by reference). Other methods include nanopore-based single-molecule real-time sequencing technology (SMRT), which is able to detect modified bases directly (described in Laszlo A. H. et al., Proc. Natl. Acad. Sci. USA. 2013 and Schreiber J., et al., Proc. Natl. Acad. Sci. USA. 2013, which are herein incorporated by reference).

### 9. Array or Bead Hybridization

Methylated DNA fractions of the genome, usually obtained by immunoprecipitation, could be used for hybridization with microarrays. Currently available examples of such arrays include: the Human CpG Island Microarray Kit (Agilent), the GeneChip Human Promoter 1.0R Array and the GeneChip Human Tiling 2.0R Array Set (Affymetrix).

The search for differentially-methylated regions using bisulfite-converted DNA could be done with the use of different techniques. Some of them are easier to perform and analyse than others, because only a fraction of the genome is used. The most pronounced functional effect of DNA methylation occurs within gene promoter regions, enhancer regulatory elements and 3′ untranslated regions (3′UTRs). Assays that focus on these specific regions, such as the Infinium HumanMethylation450 Bead Chip array by Illumina, can be used. The arrays can be used to detect methylation status of genes, including miRNA promoters, 5′ UTR, 3′ UTR, coding regions (˜17 CpG per gene) and island shores (regions ˜2 kb upstream of the CpG islands).

Briefly, bisulfite-treated genomic DNA is mixed with assay oligos, one of which is complimentary to uracil (converted from original unmethylated cytosine), and another is complimentary to the cytosine of the methylated (and therefore protected from conversion) site. Following hybridization, primers are extended and ligated to locus-specific oligos to create a template for universal PCR. Finally, labelled PCR primers are used to create detectable products that are immobilized to bar-coded beads, and the signal is measured. The ratio between two types of beads for each locus (individual CpG) is an indicator of its methylation level.

It is possible to purchase kits that utilize the extension of methylation-specific primers for validation studies. In the VeraCode Methylation assay from Illumina, 96 or 384 user-specified CpG loci are analysed with the GoldenGate Assay for Methylation. Differently from the BeadChip assay, the VeraCode assay requires the BeadXpress Reader for scanning.

### 10. Methyl-Sensitive Cut Counting: Endonuclease Digestion Followed by Sequencing

As an alternative to sequencing a substantial amount of methylated (or unmethylated) DNA, one could generate snippets from these regions and map them back to the genome after sequencing. Moreover, coverage in NGS could be good enough to quantify the methylation level for particular loci. The technique of serial analysis of gene expression (SAGE) has been adapted for this purpose and is known as methylation-specific digital karyotyping, as well as a similar technique, called methyl-sensitive cut counting (MSCC).

In summary, in all of these methods, methylation-sensitive endonuclease(s), e.g., HpaII is used for initial digestion of genomic DNA in unmethylated sites followed by adaptor ligation that contains the site for another digestion enzyme that is cut outside of its recognized site, e.g., EcoP15I or MmeI. These ways, small fragments are generated that are located in close proximity to the original HpaII site. Then, NGS and mapping to the genome are performed. The number of reads for each HpaII site correlates with its methylation level.

Recently, a number of restriction enzymes have been discovered that use methylated DNA as a substrate (methylation-dependent endonucleases). Most of them were discovered and are sold by SibEnzyme: BisI, BlsI, GlaI. GluI, KroI, MteI, PcsI, PkrI. The unique ability of these enzymes to cut only methylated sites has been utilized in the method that achieved selective amplification of methylated DNA. Three methylation-dependent endonucleases that are available from New England Biolabs (FspEI, MspJI and LpnPI) are type IIS enzymes that cut outside of the recognition site and, therefore, are able to generate snippets of 32 bp around the fully-methylated recognition site that contains CpG. These short fragments could be sequences and aligned to the reference genome. The number of reads obtained for each specific 32-bp fragment could be an indicator of its methylation level. Similarly, short fragments could be generated from methylated CpG islands with Escherichia coli's methyl-specific endonuclease McrBC, which cuts DNA between two half-sites of (G/A) mC that are lying within 50 bp-3000 bp from each other. This is a very useful tool for isolation of methylated CpG islands that again can be combined with NGS. Being bisulfite-free, these three approaches have a great potential for quick whole genome methylome profiling.

### B. Sequencing

DNA, including bisulfite-converted DNA, may be used for the amplification of a region of interest followed by sequencing. Accordingly, aspects of the disclosure may include sequencing nucleic acids to detect methylation and/or expression levels of nucleic acids and/or biomarkers. In some embodiments, the methods of the disclosure include a sequencing method. In some embodiments, the methods of the disclosure include measuring an expression level of one or more genes using a sequencing method.

In some embodiments, the disclosed methods comprise detectecting a reduced expression level of a gene (e.g., TMPRSS2, CDKN1B), for example as measured by mRNA and/or protein expression. Such methods may comprise comparing an expression level of the gene to a control or reference sample. In one example, an expression level of a gene is measured in a subject suspected of having cancer (e.g., prostate cancer) and the control or reference sample is a sample from a subject who does not have cancer (e.g., prostate cancer). Detecting a reduced expression level may comprise detecting an expression level that is at least, at most, or about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99%, 99.1%, 99.2%, 99.3%, 99.4%, 99.5%, 99.6%, 99.7%, 99.8%, or 99.9%, or any range or value derivable therein, lower than an expression level of the gene in a control or reference sample. In some embodiments, the expression level is reduced by at least 70%, 80%, or 90%. In some embodiments, the expression level is reduced by at least 90%.

In some embodiments, the disclosed methods comprise detectecting an increased expression level of a gene, for example as measured by mRNA and/or protein expression. Such methods may comprise comparing an expression level of the gene to a control or reference sample. In one example, an expression level of a gene is measured in a subject suspected of having cancer (e.g., prostate cancer) and the control or reference sample is a sample from a subject who does not have cancer (e.g., prostate cancer). Detecting an increased expression level may comprise detecting an expression level that is at least, at most, or about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99%, 99.1%, 99.2%, 99.3%, 99.4%, 99.5%, 99.6%, 99.7%, 99.8%, or 99.9%, 100%, 110%, 120%, 130%, 140%, 150%, 200%, 300%, 400%, or 500%, or any range or value derivable therein, higher than an expression level of the gene in a control or reference sample.

Example sequencing methods include those described below.

### 1. Massively Parallel Signature Sequencing (MPSS).

The first of the next-generation sequencing technologies, massively parallel signature sequencing (or MPSS), was developed in the 1990s at Lynx Therapeutics. MPSS was a bead-based method that used a complex approach of adapter ligation followed by adapter decoding, reading the sequence in increments of four nucleotides. This method made it susceptible to sequence-specific bias or loss of specific sequences. Because the technology was so complex, MPSS was only performed ‘in-house’ by Lynx Therapeutics and no DNA sequencing machines were sold to independent laboratories. Lynx Therapeutics merged with Solexa (later acquired by Illumina) in 2004, leading to the development of sequencing-by-synthesis, a simpler approach acquired from Manteia Predictive Medicine, which rendered MPSS obsolete. However, the essential properties of the MPSS output were typical of later “next-generation” data types, including hundreds of thousands of short DNA sequences. In the case of MPSS, these were typically used for sequencing cDNA for measurements of gene expression levels. Indeed, the powerful Illumina HiSeq2000, HiSeq2500 and MiSeq systems are based on MPSS.

### 2. Polony Sequencing.

The Polony sequencing method, developed in the laboratory of George M. Church at Harvard, was among the first next-generation sequencing systems and was used to sequence a full genome in 2005. It combined an in vitro paired-tag library with emulsion PCR, an automated microscope, and ligation-based sequencing chemistry to sequence an E. coli genome at an accuracy of >99.9999% and a cost approximately 1/9 that of Sanger sequencing. The technology was licensed to Agencourt Biosciences, subsequently spun out into Agencourt Personal Genomics, and eventually incorporated into the Applied Biosystems SOLiD platform, which is now owned by Life Technologies.

### 3. 454 Pyrosequencing.

A parallelized version of pyrosequencing was developed by 454 Life Sciences, which has since been acquired by Roche Diagnostics. The method amplifies DNA inside water droplets in an oil solution (emulsion PCR), with each droplet containing a single DNA template attached to a single primer-coated bead that then forms a clonal colony. The sequencing machine contains many picoliter-volume wells each containing a single bead and sequencing enzymes. Pyrosequencing uses luciferase to generate light for detection of the individual nucleotides added to the nascent DNA, and the combined data are used to generate sequence read-outs. This technology provides intermediate read length and price per base compared to Sanger sequencing on one end and Solexa and SOLiD on the other.

### 4. Illumina (Solexa) Sequencing.

Solexa, now part of Illumina, developed a sequencing method based on reversible dye-terminators technology, and engineered polymerases, that it developed internally. The terminated chemistry was developed internally at Solexa and the concept of the Solexa system was invented by Balasubramanian and Klennerman from Cambridge University's chemistry department. In 2004, Solexa acquired the company Manteia Predictive Medicine in order to gain a massivelly parallel sequencing technology based on “DNA Clusters”, which involves the clonal amplification of DNA on a surface. The cluster technology was co-acquired with Lynx Therapeutics of California. Solexa Ltd. later merged with Lynx to form Solexa Inc.

In this method, DNA molecules and primers are first attached on a slide and amplified with polymerase so that local clonal DNA colonies, later coined “DNA clusters”, are formed. To determine the sequence, four types of reversible terminator bases (RT-bases) are added and non-incorporated nucleotides are washed away. A camera takes images of the fluorescently labeled nucleotides, then the dye, along with the terminal 3′ blocker, is chemically removed from the DNA, allowing for the next cycle to begin. Unlike pyrosequencing, the DNA chains are extended one nucleotide at a time and image acquisition can be performed at a delayed moment, allowing for very large arrays of DNA colonies to be captured by sequential images taken from a single camera.

Decoupling the enzymatic reaction and the image capture allows for optimal throughput and theoretically unlimited sequencing capacity. With an optimal configuration, the ultimately reachable instrument throughput is thus dictated solely by the analog-to-digital conversion rate of the camera, multiplied by the number of cameras and divided by the number of pixels per DNA colony required for visualizing them optimally (approximately 10 pixels/colony). In 2012, with cameras operating at more than 10 MHz A/D conversion rates and available optics, fluidics and enzymatics, throughput can be multiples of 1 million nucleotides/second, corresponding roughly to one human genome equivalent at 1× coverage per hour per instrument, and one human genome re-sequenced (at approx. 30×) per day per instrument (equipped with a single camera).

### 5. Solid Sequencing.

Applied Biosystems' (now a Thermo Fisher Scientific brand) SOLiD technology employs sequencing by ligation. Here, a pool of all possible oligonucleotides of a fixed length are labeled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting beads, each containing single copies of the same DNA molecule, are deposited on a glass slide. The result is sequences of quantities and lengths comparable to Illumina sequencing. This sequencing by ligation method has been reported to have some issue sequencing palindromic sequences.

### 6. Ion Torrent Semiconductor Sequencing.

Ion Torrent Systems Inc. (now owned by Thermo Fisher Scientific) developed a system based on using standard sequencing chemistry, but with a novel, semiconductor based detection system. This method of sequencing is based on the detection of hydrogen ions that are released during the polymerization of DNA, as opposed to the optical methods used in other sequencing systems. A microwell containing a template DNA strand to be sequenced is flooded with a single type of nucleotide. If the introduced nucleotide is complementary to the leading template nucleotide it is incorporated into the growing complementary strand. This causes the release of a hydrogen ion that triggers a hypersensitive ion sensor, which indicates that a reaction has occurred. If homopolymer repeats are present in the template sequence multiple nucleotides will be incorporated in a single cycle. This leads to a corresponding number of released hydrogens and a proportionally higher electronic signal.

### 7. DNA Nanoball Sequencing.

DNA nanoball sequencing is a type of high throughput sequencing technology used to determine the entire genomic sequence of an organism. The company Complete Genomics uses this technology to sequence samples submitted by independent researchers. The method uses rolling circle replication to amplify small fragments of genomic DNA into DNA nanoballs. Unchained sequencing by ligation is then used to determine the nucleotide sequence. This method of DNA sequencing allows large numbers of DNA nanoballs to be sequenced per run and at low reagent costs compared to other next generation sequencing platforms. However, only short sequences of DNA are determined from each DNA nanoball which makes mapping the short reads to a reference genome difficult. This technology has been used for multiple genome sequencing projects.

### 8. Heliscope Single Molecule Sequencing.

Heliscope sequencing is a method of single-molecule sequencing developed by Helicos Biosciences. It uses DNA fragments with added poly-A tail adapters which are attached to the flow cell surface. The next steps involve extension-based sequencing with cyclic washes of the flow cell with fluorescently labeled nucleotides (one nucleotide type at a time, as with the Sanger method). The reads are performed by the Heliscope sequencer. The reads are short, up to 55 bases per run, but recent improvements allow for more accurate reads of stretches of one type of nucleotides. This sequencing method and equipment were used to sequence the genome of the M13 bacteriophage.

### 9. Single Molecule Real Time (SMRT) Sequencing.

SMRT sequencing is based on the sequencing by synthesis approach. The DNA is synthesized in zero-mode wave-guides (ZMWs)—small well-like containers with the capturing tools located at the bottom of the well. The sequencing is performed with use of unmodified polymerase (attached to the ZMW bottom) and fluorescently labelled nucleotides flowing freely in the solution. The wells are constructed in a way that only the fluorescence occurring by the bottom of the well is detected. The fluorescent label is detached from the nucleotide at its incorporation into the DNA strand, leaving an unmodified DNA strand. According to Pacific Biosciences, the SMRT technology developer, this methodology allows detection of nucleotide modifications (such as cytosine methylation). This happens through the observation of polymerase kinetics. This approach allows reads of 20,000 nucleotides or more, with average read lengths of 5 kilobases.

### C. Additional Assay Methods

In some embodiments, methods involve amplifying and/or sequencing one or more target genomic regions using at least one pair of primers specific to the target genomic regions. In certain embodiments, the primers are heptamers. In other embodiments, enzymes are added such as primases or primase/polymerase combination enzyme to the amplification step to synthesize primers.

In some embodiments, arrays can be used to detect nucleic acids of the disclosure. An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., 1991), each of which is incorporated by reference in its entirety for all purposes. Techniques for the synthesis of these arrays using mechanical synthesis methods are described in, e.g., U.S. Pat. No. 5,384,261, incorporated herein by reference in its entirety for all purposes. Although a planar array surface is used in certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, which are hereby incorporated in their entirety for all purposes.

In addition to the use of arrays and microarrays, it is contemplated that a number of difference assays could be employed to analyze nucleic acids. Such assays include, but are not limited to, nucleic amplification, polymerase chain reaction, quantitative PCR, RT-PCR, in situ hybridization, digital PCR, dd PCR (digital droplet PCR), nCounter (nanoString), BEAMing (Beads, Emulsions, Amplifications, and Magnetics) (Inostics), ARMS (Amplification Refractory Mutation Systems), RNA-Seq, TAm-Seg (Tagged-Amplicon deep sequencing), PAP (Pyrophosphorolysis-activation polymerization), next generation RNA sequencing, northern hybridization, hybridization protection assay (HPA)(GenProbe), branched DNA (bDNA) assay (Chiron), rolling circle amplification (RCA), single molecule hybridization detection (US Genomics), Invader assay (ThirdWave Technologies), and/or Bridge Litigation Assay (Genaco).

Amplification primers or hybridization probes can be prepared to be complementary to a genomic region, biomarker, probe, or oligo described herein. The term “primer” or “probe” as used herein, is meant to encompass any nucleic acid that is capable of priming the synthesis of a nascent nucleic acid in a template-dependent process and/or pairing with a single strand of an oligo of the disclosure, or portion thereof. Typically, primers are oligonucleotides from ten to twenty and/or thirty nucleic acids in length, but longer sequences can be employed. Primers may be provided in double-stranded and/or single-stranded form, although the single-stranded form is preferred.

The use of a probe or primer of between 13 and 100 nucleotides, particularly between 17 and 100 nucleotides in length, or in some aspects up to 1-2 kilobases or more in length, allows the formation of a duplex molecule that is both stable and selective. Molecules having complementary sequences over contiguous stretches greater than 20 bases in length may be used to increase stability and/or selectivity of the hybrid molecules obtained. One may design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

In one embodiment, each probe/primer comprises at least 15 nucleotides. For instance, each probe can comprise at least or at most 20, 25, 50, 75, 100, 125, 150, 175, 200, 225, 250, 275, 300, 325, 350, 400 or more nucleotides (or any range derivable therein). They may have these lengths and have a sequence that is identical or complementary to a gene described herein. Particularly, each probe/primer has relatively high sequence complexity and does not have any ambiguous residue (undetermined “n” residues). The probes/primers can hybridize to the target gene, including its RNA transcripts, under stringent or highly stringent conditions. It is contemplated that probes or primers may have inosine or other design implementations that accommodate recognition of more than one human sequence for a particular biomarker.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

In one embodiment, quantitative RT-PCR (such as TaqMan, ABI) is used for detecting and comparing the levels or abundance of nucleic acids in samples. The concentration of the target DNA in the linear portion of the PCR process is proportional to the starting concentration of the target before the PCR was begun. By determining the concentration of the PCR products of the target DNA in PCR reactions that have completed the same number of cycles and are in their linear ranges, it is possible to determine the relative concentrations of the specific target sequence in the original DNA mixture. This direct proportionality between the concentration of the PCR products and the relative abundances in the starting material is true in the linear range portion of the PCR reaction. The final concentration of the target DNA in the plateau portion of the curve is determined by the availability of reagents in the reaction mix and is independent of the original concentration of target DNA. Therefore, the sampling and quantifying of the amplified PCR products may be carried out when the PCR reactions are in the linear portion of their curves. In addition, relative concentrations of the amplifiable DNAs may be normalized to some independent standard/control, which may be based on either internally existing DNA species or externally introduced DNA species. The abundance of a particular DNA species may also be determined relative to the average abundance of all DNA species in the sample.

In one embodiment, the PCR amplification utilizes one or more internal PCR standards. The internal standard may be an abundant housekeeping gene in the cell or it can specifically be GAPDH, GUSB and β-2 microglobulin. These standards may be used to normalize expression levels so that the expression levels of different gene products can be compared directly. A person of ordinary skill in the art would know how to use an internal standard to normalize expression levels.

A problem inherent in some samples is that they are of variable quantity and/or quality. This problem can be overcome if the RT-PCR is performed as a relative quantitative RT-PCR with an internal standard in which the internal standard is an amplifiable DNA fragment that is similar or larger than the target DNA fragment and in which the abundance of the DNA representing the internal standard is roughly 5-100 fold higher than the DNA representing the target nucleic acid region.

In another embodiment, the relative quantitative RT-PCR uses an external standard protocol. Under this protocol, the PCR products are sampled in the linear portion of their amplification curves. The number of PCR cycles that are optimal for sampling can be empirically determined for each target DNA fragment. In addition, the nucleic acids isolated from the various samples can be normalized for equal concentrations of amplifiable DNAs.

A nucleic acid array can comprise at least 1, 2, 3, 4, 5, 6, 7, 8, 9, 10, 15, 20, 25, 30, 35, 40, 45, 50, 60, 70, 80, 90, 100, 150, 200, 250 or more different polynucleotide probes, which may hybridize to different and/or the same biomarkers. Multiple probes for the same gene can be used on a single nucleic acid array. Probes for other disease genes can also be included in the nucleic acid array. The probe density on the array can be in any range. In some embodiments, the density may be or may be at least 50, 100, 200, 300, 400, 500 or more probes/cm2 (or any range derivable therein).

Specifically contemplated are chip-based nucleic acid technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see also, Pease et al., 1994; and Fodor et al, 1991). It is contemplated that this technology may be used in conjunction with evaluating the expression level of one or more cancer biomarkers with respect to diagnostic, prognostic, and treatment methods.

Certain embodiments may involve the use of arrays or data generated from an array. Data may be readily available. Moreover, an array may be prepared in order to generate data that may then be used in correlation studies.

### V. Administration of Therapeutic Compositions

The therapy provided herein may comprise administration of a combination of therapeutic agents, such as a first cancer therapy and a second cancer therapy. The therapies may be administered in any suitable manner known in the art. For example, the first and second cancer treatment may be administered sequentially (at different times) or concurrently (at the same time). In some embodiments, the first and second cancer treatments are administered in a separate composition. In some embodiments, the first and second cancer treatments are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 M or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### VI. Kits

Certain aspects of the present invention also concern kits containing compositions of the disclosure or compositions to implement methods of the disclosure. In some embodiments, kits can be used to evaluate one or more biomarkers. In some embodiments, kits can be used to determine the presence of one or more polymorphisms (e.g., SNPs). In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein (e.g., one or more SNPs listed in Table 1), which includes nucleic acid primers/primer sets and probes that are identical to or complementary to all or part of a biomarker.

In certain aspects, negative and/or positive control nucleic acids, probes, and inhibitors are included in some kit embodiments. In addition, a kit may include a sample that is a negative or positive control for one or more biomarkers.

Embodiments of the disclosure include kits for analysis of a pathological sample by assessing biomarker profile for a sample comprising, in suitable container means, two or more biomarker probes, wherein the biomarker probes detect one or more of the biomarkers identified herein. The kit can further comprise reagents for labeling nucleic acids in the sample. The kit may also include labeling reagents, including at least one of amine-modified nucleotide, poly(A) polymerase, and poly(A) polymerase buffer. Labeling reagents can include an amine-reactive dye.

### VII. Detecting a Genetic Signature

Particular embodiments concern the methods of detecting a genetic signature in an individual. In some embodiments, the method for detecting the genetic signature may include selective oligonucleotide probes, arrays, allele-specific hybridization, molecular beacons, restriction fragment length polymorphism analysis, enzymatic chain reaction, flap endonuclease analysis, primer extension, 5′-nuclease analysis, oligonucleotide ligation assay, single strand conformation polymorphism analysis, temperature gradient gel electrophoresis, denaturing high performance liquid chromatography, high-resolution melting, DNA mismatch binding protein analysis, surveyor nuclease assay, sequencing, or a combination thereof, for example. The method for detecting the genetic signature may include fluorescent in situ hybridization, comparative genomic hybridization, arrays, polymerase chain reaction, sequencing, or a combination thereof, for example. The detection of the genetic signature may involve using a particular method to detect one feature of the genetic signature and additionally use the same method or a different method to detect a different feature of the genetic signature. Multiple different methods independently or in combination may be used to detect the same feature or a plurality of features.

### A. Single Nucleotide Polymorphism (SNP) Detection

Particular embodiments of the disclosure concern methods of detecting a SNP in an individual. One may employ any of the known general methods for detecting SNPs for detecting the particular SNP in this disclosure, for example. Such methods include, but are not limited to, selective oligonucleotide probes, arrays, allele-specific hybridization, molecular beacons, restriction fragment length polymorphism analysis, enzymatic chain reaction, flap endonuclease analysis, primer extension, 5′-nuclease analysis, oligonucleotide ligation assay, single strand conformation polymorphism analysis, temperature gradient gel electrophoresis, denaturing high performance liquid chromatography, high-resolution melting, DNA mismatch binding protein analysis, surveyor nuclease assay, sequencing, and a combination thereof.

In some embodiments of the disclosure, the method used to detect the SNP comprises sequencing nucleic acid material from the individual and/or using selective oligonucleotide probes. Sequencing the nucleic acid material from the individual may involve obtaining the nucleic acid material from the individual in the form of genomic DNA, complementary DNA that is reverse transcribed from RNA, or RNA, for example. Any standard sequencing technique may be employed, including Sanger sequencing, chain extension sequencing, Maxam-Gilbert sequencing, shotgun sequencing, bridge PCR sequencing, high-throughput methods for sequencing, next generation sequencing, RNA sequencing, or a combination thereof. After sequencing the nucleic acid from the individual, one may utilize any data processing software or technique to determine which particular nucleotide is present in the individual at the particular SNP.

In some embodiments, the nucleotide at the particular SNP is detected by selective oligonucleotide probes. The probes may be used on nucleic acid material from the individual, including genomic DNA, complementary DNA that is reverse transcribed from RNA, or RNA, for example. Selective oligonucleotide probes preferentially bind to a complementary strand based on the particular nucleotide present at the SNP. For example, one selective oligonucleotide probe binds to a complementary strand that has an A nucleotide at the SNP on the coding strand but not a G nucleotide at the SNP on the coding strand, while a different selective oligonucleotide probe binds to a complementary strand that has a G nucleotide at the SNP on the coding strand but not an A nucleotide at the SNP on the coding strand. Similar methods could be used to design a probe that selectively binds to the coding strand that has a C or a T nucleotide, but not both, at the SNP. Thus, any method to determine binding of one selective oligonucleotide probe over another selective oligonucleotide probe could be used to determine the nucleotide present at the SNP.

One method for detecting SNPs using oligonucleotide probes comprises the steps of analyzing the quality and measuring quantity of the nucleic acid material by a spectrophotometer and/or a gel electrophoresis assay; processing the nucleic acid material into a reaction mixture with at least one selective oligonucleotide probe, PCR primers, and a mixture with components needed to perform a quantitative PCR (qPCR), which could comprise a polymerase, deoxynucleotides, and a suitable buffer for the reaction; and cycling the processed reaction mixture while monitoring the reaction. In one embodiment of the method, the polymerase used for the qPCR will encounter the selective oligonucleotide probe binding to the strand being amplified and, using endonuclease activity, degrade the selective oligonucleotide probe. The detection of the degraded probe determines if the probe was binding to the amplified strand.

Another method for determining binding of the selective oligonucleotide probe to a particular nucleotide comprises using the selective oligonucleotide probe as a PCR primer, wherein the selective oligonucleotide probe binds preferentially to a particular nucleotide at the SNP position. In some embodiments, the probe is generally designed so the 3′ end of the probe pairs with the SNP. Thus, if the probe has the correct complementary base to pair with the particular nucleotide at the SNP, the probe will be extended during the amplification step of the PCR. For example, if there is a T nucleotide at the 3′ position of the probe and there is an A nucleotide at the SNP position, the probe will bind to the SNP and be extended during the amplification step of the PCR. However, if the same probe is used (with a T at the 3′ end) and there is a G nucleotide at the SNP position, the probe will not fully bind and will not be extended during the amplification step of the PCR.

In some embodiments, the SNP position is not at the terminal end of the PCR primer, but rather located within the PCR primer. The PCR primer should be of sufficient length and homology in that the PCR primer can selectively bind to one variant, for example the SNP having an A nucleotide, but not bind to another variant, for example the SNP having a G nucleotide. The PCR primer may also be designed to selectively bind particularly to the SNP having a G nucleotide but not bind to a variant with an A, C, or T nucleotide. Similarly, PCR primers could be designed to bind to the SNP having a C or a T nucleotide, but not both, which then does not bind to a variant with a G, A, or T nucleotide or G, A, or C nucleotide respectively. In particular embodiments, the PCR primer is at least or no more than 10, 11, 12, 13, 14, 15, 16, 17, 18, 19, 20, 21, 22, 23, 24, 25, 26, 27, 28, 29, 30, 31, 32, 33, 34,3 5, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or more nucleotides in length with 100% homology to the template sequence, with the potential exception of non-homology the SNP location. After several rounds of amplifications, if the PCR primers generate the expected band size, the SNP can be determined to have the A nucleotide and not the G nucleotide.

As described herein, a subject may be or have been genotyped as having one or more SNPs. For example, a subject may be or have been genotyped as having one or more SNPs listed in Table 1. SNPs disclosed herein may be described by one or more designations.

In some embodiments, a SNP is designated by a chromosomal location and one or more nucleotides. For example, a subject may be described as having the SNP chr8:127535470 T>A. In this example, such a description identifies the subject as having an A nucleotide (instead of the more common T nucleotide) at the chromosomal location chr8:127535470. In another example, a subject may be described as having the SNP chr13:49282062 C>A,T. In this example, such a description identifies the subject as having either an A nucleotide or a T nucleotide (instead of the more common C nucleotide) at the chromosomal location chr13:49282062.

In some embodiments, a SNP is designated by a Reference SNP (also “RefSNP” or “rs”) identifier. When a subject is described herein as having a particular SNP by use of an rs identifier, such a description is understood to encompass any nucleotide or sequence encompassed by the rs idenfieier. An rs identifier for a SNP may encompass one single nucleotide or may encompass two or more alternative nucleotides (i.e. two or more “alleles”). For example, a subject genotyped as having the SNP rs111620024 describes a subject having a T allele at chromosomal position chr5:96662687, while a subject genotyped as having the SNP rs12653946 describes a subject having either an A or a T at chromosomal location chr5:1895715. Databases harboring information regarding SNPs (and other genomic variants) are known to the skilled artisan and include, for example, the Single Nucleotide Polymorphism Database (dbSNP), available on the World Wide Web at ncbi.nlm.nih.gov/snp, described in Smigielski E M, et al,. dbSNP: a database of single nucleotide polymorphisms. Nucleic Acids Res. 2000 Jan. 1; 28(1):352-5, incorporated herein by reference in its entirety.

### B. Copy Number Variation Detection

Particular embodiments of the disclosure concern methods of detecting a copy number variation (CNV) of a particular allele. One can utilize any known method for detecting CNVs to detect the CNVs. Such methods include fluorescent in situ hybridization, comparative genomic hybridization, arrays, polymerase chain reaction, sequencing, or a combination thereof, for example. In some embodiments, the CNV is detected using an array. Array platforms such as those from Agilent, Illumina, or Affymetrix may be used, or custom arrays could be designed. One example of how an array may be used includes methods that comprise one or more of the steps of isolating nucleic acid material in a suitable manner from an individual suspected of having the CNV and, at least in some cases from an individual or reference genome that does not have the CNV; processing the nucleic acid material by fragmentation, labelling the nucleic acid with, for example, fluorescent labels, and purifying the fragmented and labeled nucleic acid material; hybridizing the nucleic acid material to the array for a sufficient time, such as for at least 24 hours; washing the array after hybridization; scanning the array using an array scanner; and analyzing the array using suitable software. The software may be used to compare the nucleic acid material from the individual suspected of having the CNV to the nucleic acid material of an individual who is known not to have the CNV or a reference genome.

In some embodiments, detection of a CNV is achieved by polymerase chain reaction (PCR). PCR primers can be employed to amplify nucleic acid at or near the CNV wherein an individual with a CNV will result in measurable higher levels of PCR product when compared to a PCR product from a reference genome. The detection of PCR product amounts could be measured by quantitative PCR (qPCR) or could be measured by gel electrophoresis, as examples. Quantification using gel electrophoresis comprises subjecting the resulting PCR product, along with nucleic acid standards of known size, to an electrical current on an agarose gel and measuring the size and intensity of the resulting band. The size of the resulting band can be compared to the known standards to determine the size of the resulting band. In some embodiments, the amplification of the CNV will result in a band that has a larger size than a band that is amplified, using the same primers as were used to detect the CNV, from a reference genome or an individual that does not have the CNV being detected. The resulting band from the CNV amplification may be nearly double, double, or more than double the resulting band from the reference genome or the resulting band from an individual that does not have the CNV being detected. In some embodiments, the CNV can be detected using nucleic acid sequencing. Sequencing techniques that could be used include, but are not limited to, whole genome sequencing, whole exome sequencing, and/or targeted sequencing.

### C. DNA Sequencing

In some embodiments, DNA may be analyzed by sequencing. The DNA may be prepared for sequencing by any method known in the art, such as library preparation, hybrid capture, sample quality control, product-utilized ligation-based library preparation, or a combination thereof. The DNA may be prepared for any sequencing technique. In some embodiments, a unique genetic readout for each sample may be generated by genotyping one or more highly polymorphic SNPs. In some embodiments, sequencing, such as 76 base pair, paired-end sequencing, may be performed to cover approximately 70%, 75%, 80%, 85%, 90%, 95%, 99%, or greater percentage of targets at more than 20×, 25×, 30×, 35×, 40×, 45×, 50×, or greater than 50× coverage. In certain embodiments, mutations, SNPS, INDELS, copy number alterations (somatic and/or germline), or other genetic differences may be identified from the sequencing using at least one bioinformatics tool, including VarScan2, any R package (including CopywriteR) and/or Annovar.

### D. RNA Sequencing

In some embodiments, RNA may be analyzed by sequencing. The RNA may be prepared for sequencing by any method known in the art, such as poly-A selection, cDNA synthesis, stranded or nonstranded library preparation, or a combination thereof. The RNA may be prepared for any type of RNA sequencing technique, including stranded specific RNA sequencing. In some embodiments, sequencing may be performed to generate approximately 10M, 15M, 20M, 25M, 30M, 35M, 40M or more reads, including paired reads. The sequencing may be performed at a read length of approximately 50 bp, 55 bp, 60 bp, 65 bp, 70 bp, 75 bp, 80 bp, 85 bp, 90 bp, 95 bp, 100 bp, 105 bp, 110 bp, or longer. In some embodiments, raw sequencing data may be converted to estimated read counts (RSEM), fragments per kilobase of transcript per million mapped reads (FPKM), and/or reads per kilobase of transcript per million mapped reads (RPKM). In some embodiments, one or more bioinformatics tools may be used to infer stroma content, immune infiltration, and/or tumor immune cell profiles, such as by using upper quartile normalized RSEM data.

### E. Proteomics

In some embodiments, protein may be analyzed by mass spectrometry. The protein may be prepared for mass spectrometry using any method known in the art. Protein, including any isolated protein encompassed herein, may be treated with DTT followed by iodoacetamide. The protein may be incubated with at least one peptidase, including an endopeptidase, proteinase, protease, or any enzyme that cleaves proteins. In some embodiments, protein is incubated with the endopeptidase, LysC and/or trypsin. The protein may be incubated with one or more protein cleaving enzymes at any ratio, including a ratio of μg of enzyme to μg protein at approximately 1:1000, 1:100, 1:90, 1:80, 1:70, 1:60, 1:50, 1:40, 1:30, 1:20, 1:10, 1:1, or any range between. In some embodiments, the cleaved proteins may be purified, such as by column purification. In certain embodiments, purified peptides may be snap-frozen and/or dried, such as dried under vacuum. In some embodiments, the purified peptides may be fractionated, such as by reverse phase chromatography or basic reverse phase chromatography. Fractions may be combined for practice of the methods of the disclosure. In some embodiments, one or more fractions, including the combined fractions, are subject to phosphopeptide enrichment, including phospho-enrichment by affinity chromatography and/or binding, ion exchange chromatography, chemical derivatization, immunoprecipitation, co-precipitation, or a combination thereof. The entirety or a portion of one or more fractions, including the combined fractions and/or phospho-enriched fractions, may be subject to mass spectrometry. In some embodiments, the raw mass spectrometry data may be processed and normalized using at least one relevant bioinformatics tool.

### F. Detection Kits and Systems

One can recognize that based on the methods described herein, detection reagents, kits, and/or systems can be utilized to detect a SNP and/or CNV related to the genetic signature for diagnosing or prognosing an individual (the detection either individually or in combination). The reagents can be combined into at least one of the established formats for kits and/or systems as known in the art. As used herein, the terms “kits” and “systems” refer to embodiments such as combinations of at least one SNP detection reagent, for example at least one selective oligonucleotide probe, and at least one CNV detection reagent, for example at least one PCR primer. The kits could also contain other reagents, chemicals, buffers, enzymes, packages, containers, electronic hardware components, etc. The kits/systems could also contain packaged sets of PCR primers, oligonucleotides, arrays, beads, or other detection reagents. Any number of probes could be implemented for a detection array. In some embodiments, the detection reagents and/or the kits/systems are paired with chemiluminescent or fluorescent detection reagents. Particular embodiments of kits/systems include the use of electronic hardware components, such as DNA chips or arrays, or microfluidic systems, for example. In specific embodiments, the kit also comprises one or more therapeutic or prophylactic interventions in the event the individual is determined to be in need of.

### EXAMPLES

The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute certain modes for its practice. However, those of skill in the art should, in light of the present disclosure, appreciate that many changes can be made in the specific embodiments which are disclosed and still obtain a like or similar result without departing from the spirit and scope of the invention.

### Example 1—Germline Determinants of the Prostate Tumor Genome

The inventors quantified the relationships between germline SNPs and somatic mutational profiles in prostate cancer. Tumors with elevated genetic risk harbor fewer somatic mutations and fewer driver mutations, analogous to a “polygenic two-hit” model²¹, where higher-risk genetic background permits tumor initiation with fewer mutations. Individual SNPs, termed driver quantitative trait loci (dQTL), influence acquisition of specific prostate cancer driver mutations. Integrating linear and three-dimensional analysis of DNA structure, the inventors identify 62 dQTLs affecting 20 driver genes. These dQTLs influence prostate cancer methylation, chromatin structure, mRNA abundance, protein abundance, grade at diagnosis and risk of relapse after definitive local therapy. Some dQTLs were active in multiple cancer types. Specific dQTLs associated with somatic TMPRSS2-ERG fusion and FOXA1 point mutations explain large fractions of observed differences in mutation frequencies across ancestry groups. To show how the molecular features of a cancer may be predicted decades prior to diagnosis, the inventors built a driver polygenic risk score (dPRS) that accurately predicted whether a tumor will harbor a TMPRSS2-ERG fusion.

### Experimental and Cohort Design

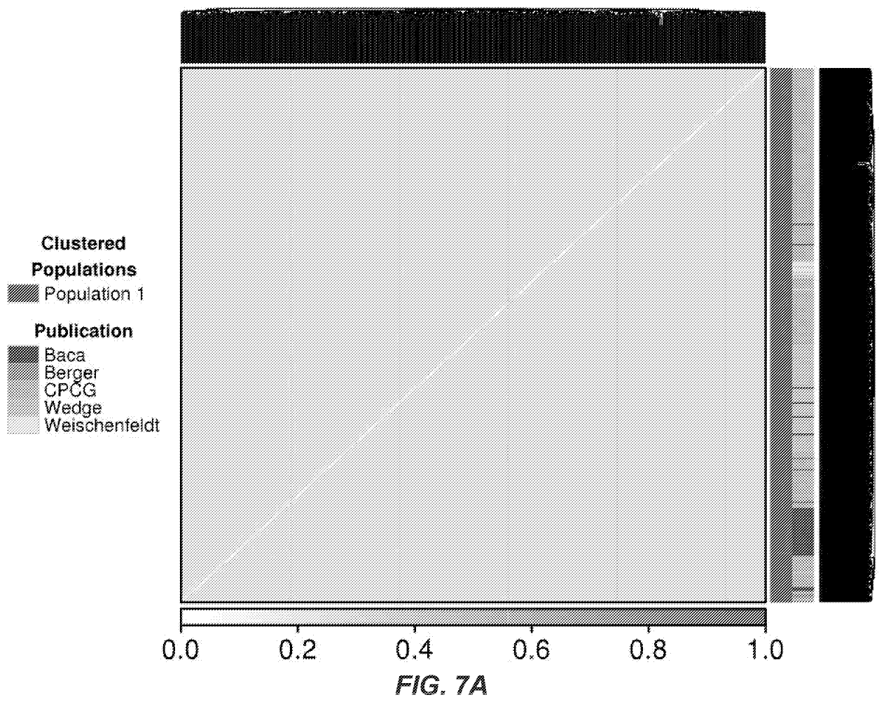

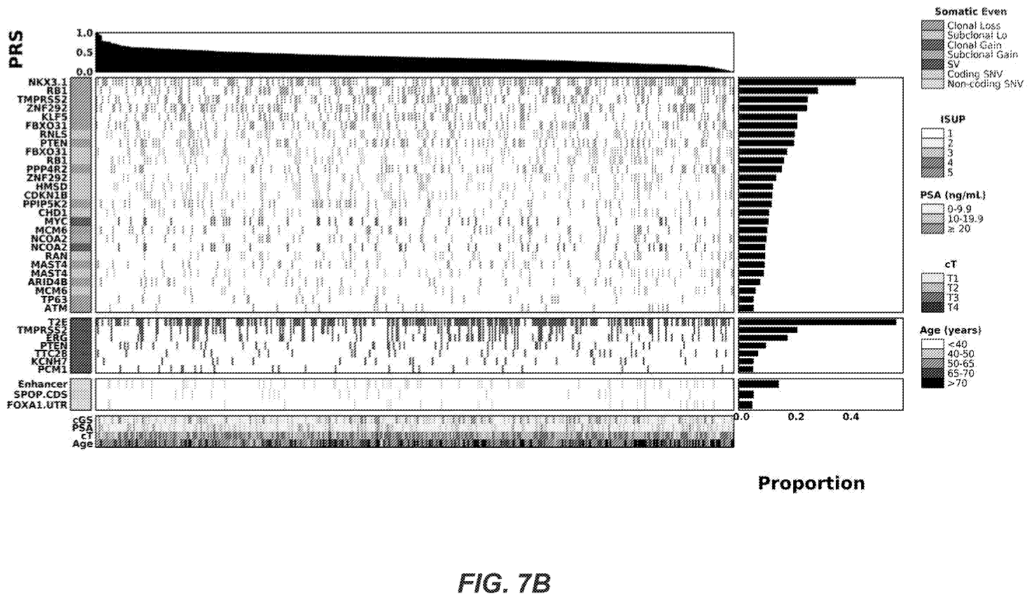

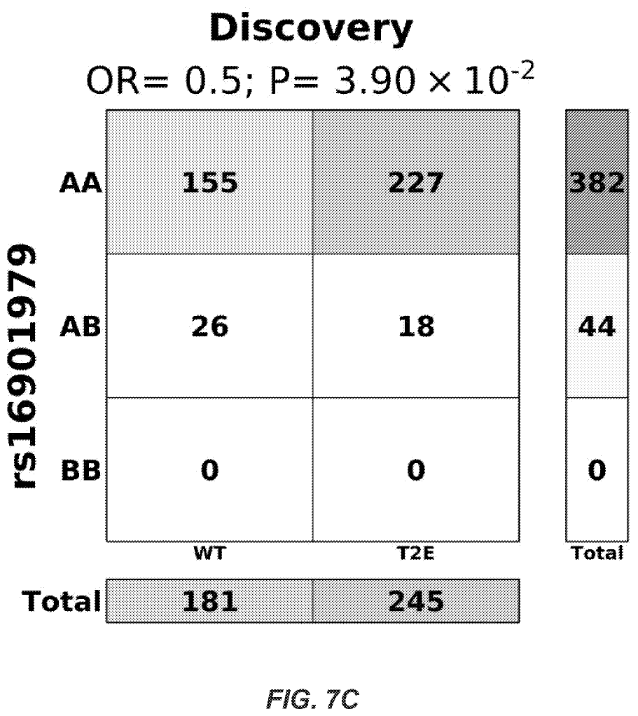

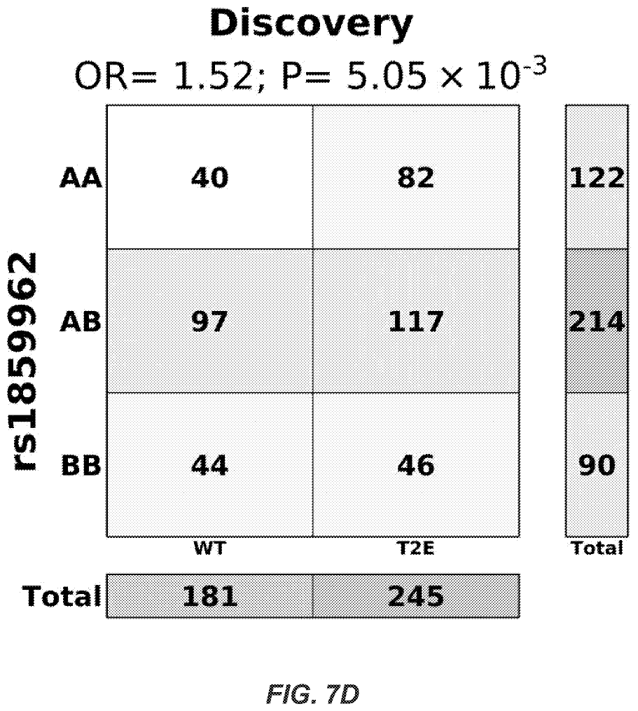

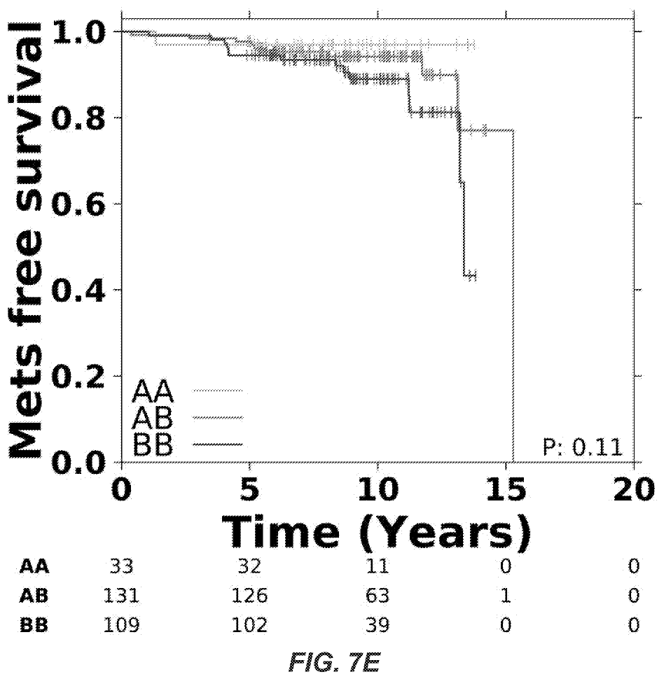

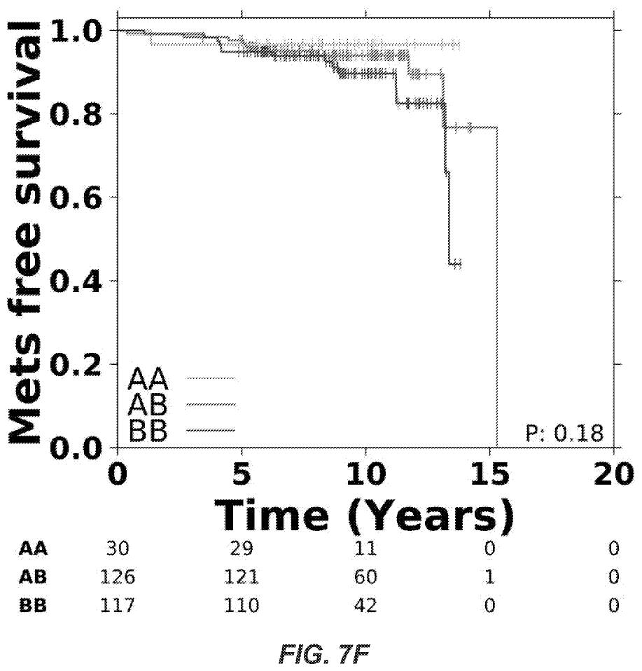

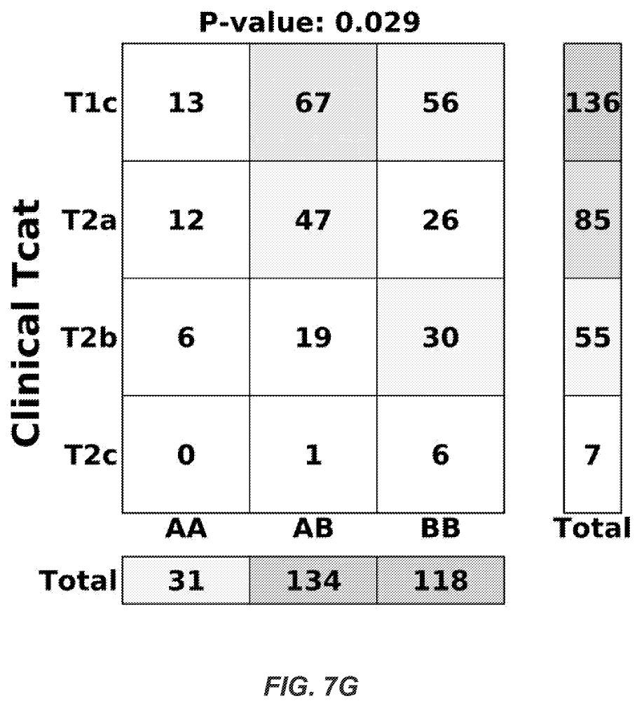

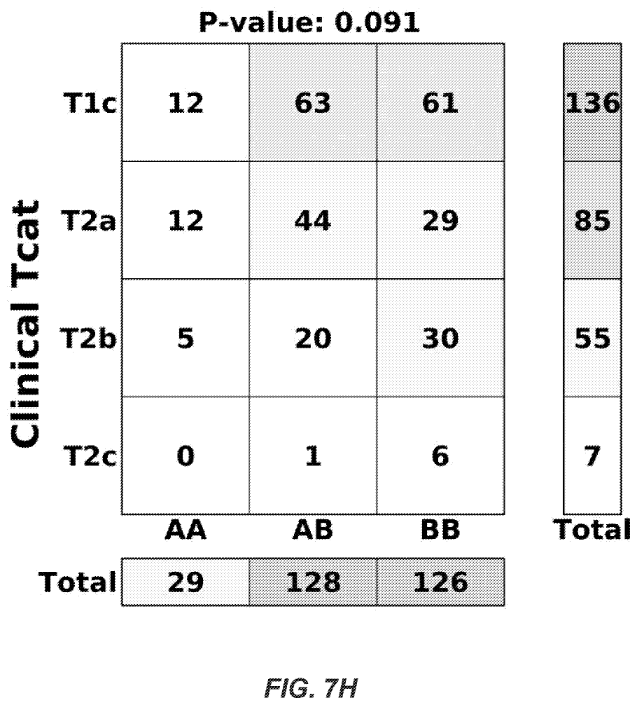

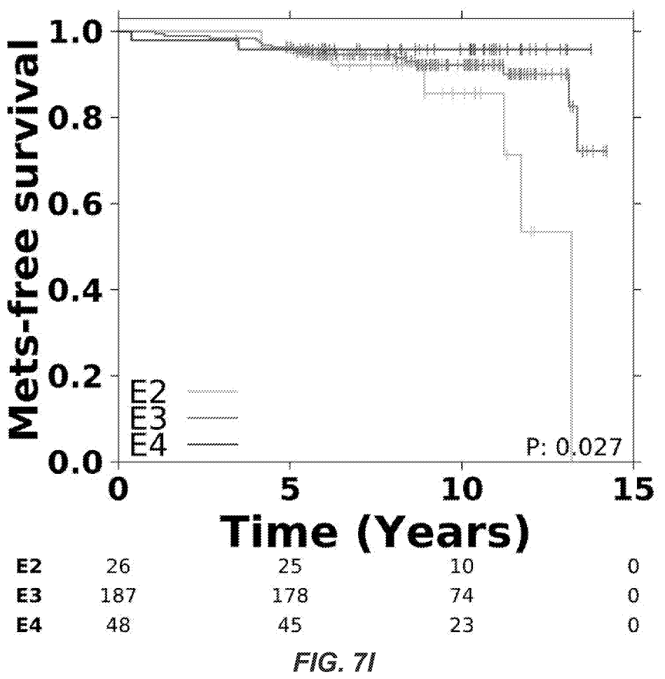

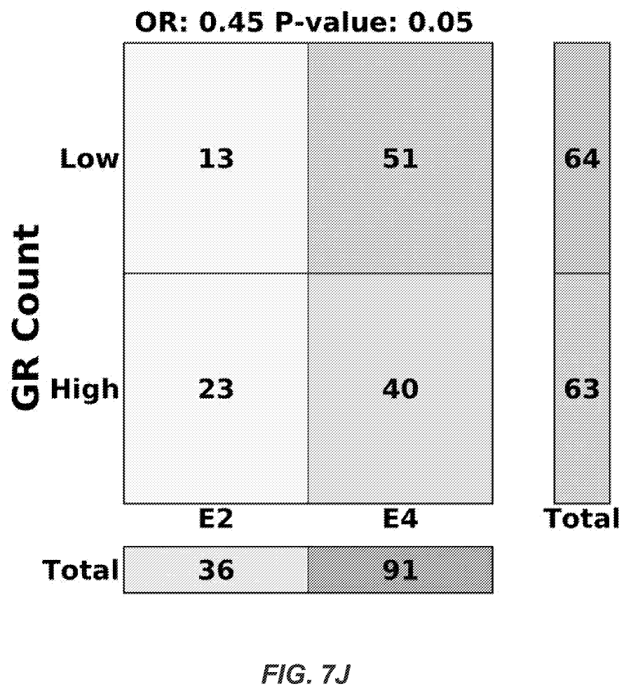

The inventors assembled a discovery cohort of 427 patients with localized prostate cancer, each with whole-genome sequencing (WGS) of blood (mean 39× coverage) and tumor (mean 64× coverage)²²⁻²⁶. All patients had localized disease at diagnosis and were treated by image-guided radiotherapy or surgery with curative intent. All samples were treatment-naïve, and were reviewed by a genitourinary pathologist and macro-dissected to obtain 70% tumor cellularity, as verified computationally²⁶. Median follow-up was 7.67 years. Patients were of European ancestry and identity-by-state clustering did not reveal population substructure (FIG. 7A). Sequencing data were uniformly processed from read-level using benchmarked pipelines^{27,28}. The inventors identified 37 somatic drivers occurring in at least 5% of patients based on enrichment over the local background mutational rate and support for the literature (range: 5.1-57.1%; FIG. 7B). These comprised 27 copy number aberrations (CNAs), 3 single nucleotide variants (SNVs) and 7 genomic rearrangements (GRs)²⁶. CNAs were sub-categorized by presence in all tumor cells (i.e. clonal) vs. a subset (i.e. subclonal).

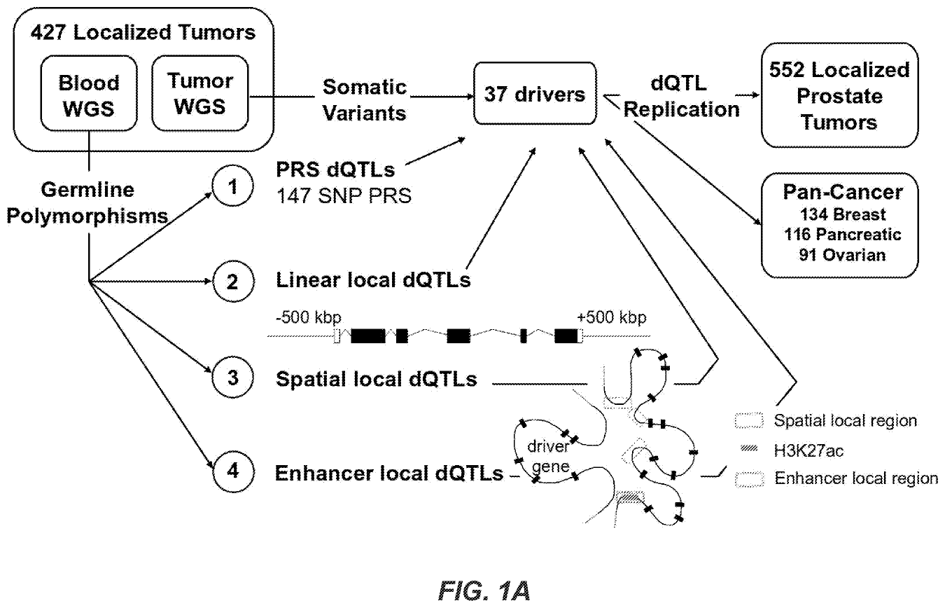

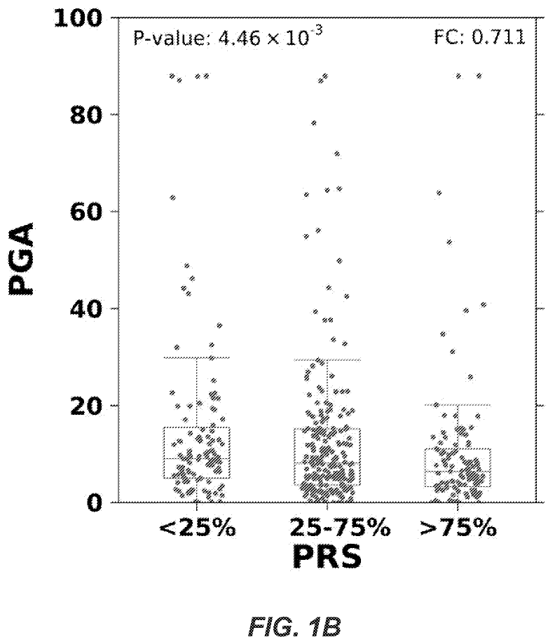

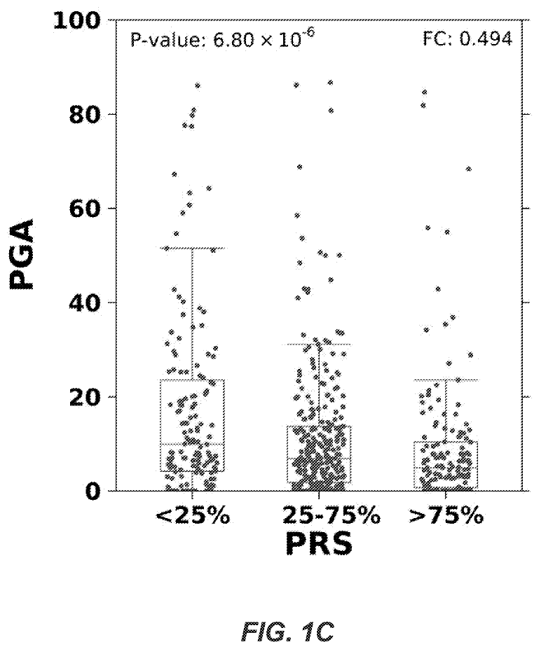

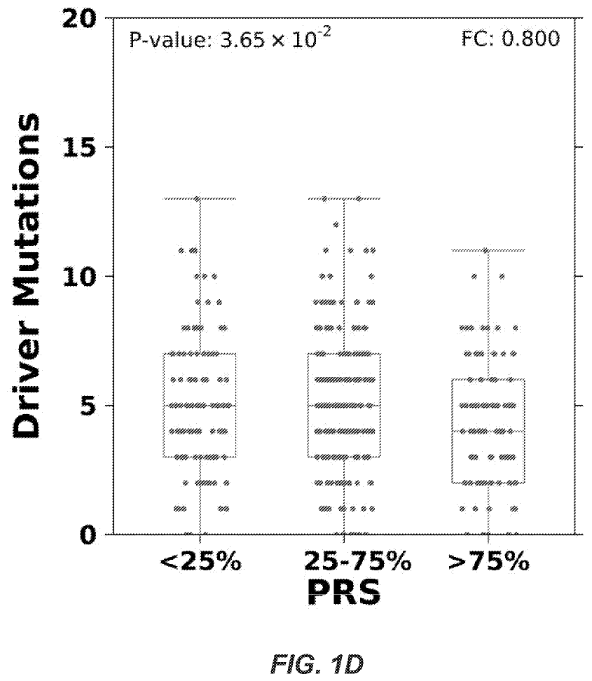

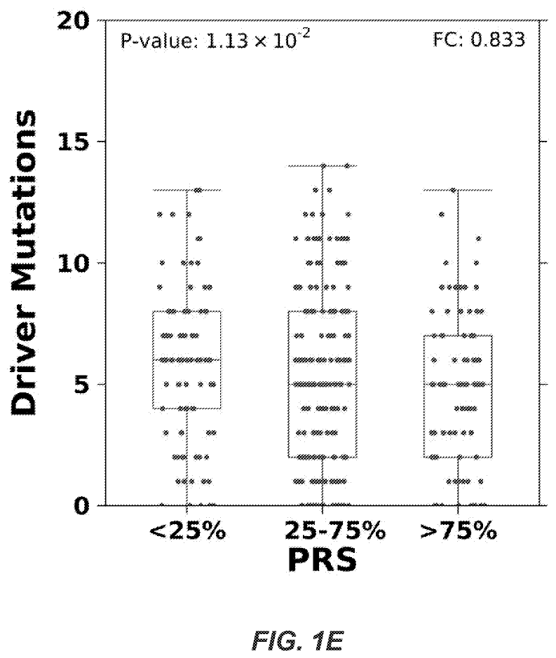

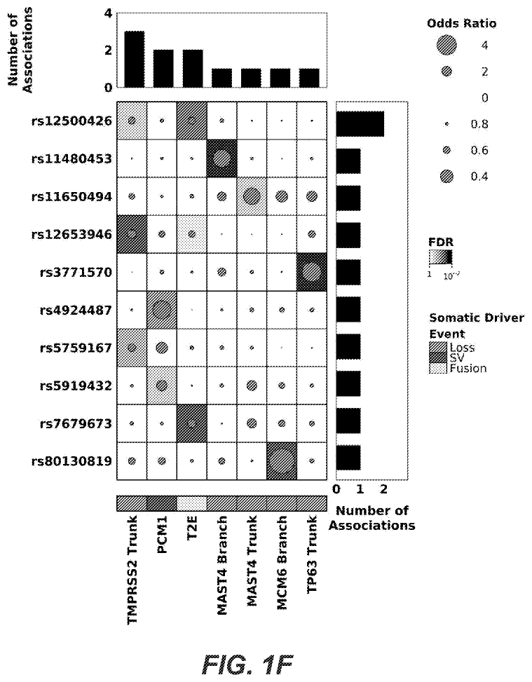

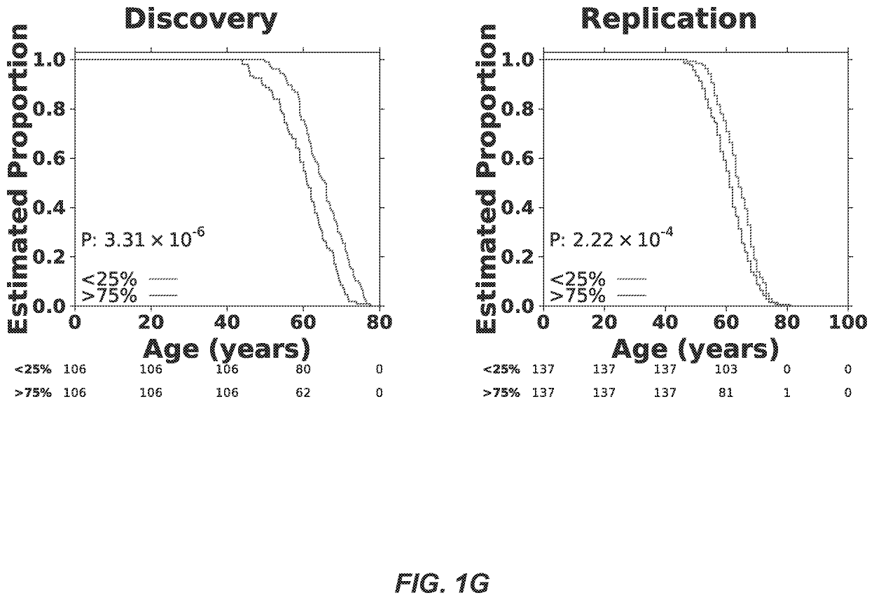

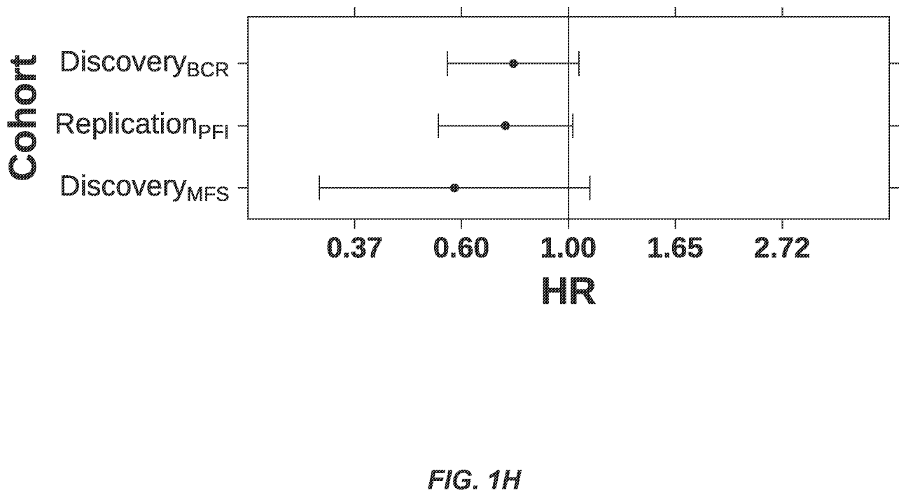

The inventors sought to determine if individual germline SNPs are associated with specific driver mutations; described herein as driver quantitative trait loci (dQTLs). A fully-powered genome-wide discovery will require many thousands of patients with tumor whole-genome sequencing. The inventors therefore sought to enrich for dQTLs with three complementary biologically-motivated approaches (FIG. 1A). First, the inventors tested if germline SNPs associated with risk of diagnosis in GWAS studies are dQTLs. Second, the inventors identified local dQTLs: regions in close proximity to each somatic driver based on linear DNA sequence. Third, the inventors exploited knowledge of three-dimensional DNA structure to identify spatial local dQTLs: regions proximal to somatic drivers through chromatin structure. Altogether, 18,414 independent SNPs were evaluated across all 37 somatic drivers.

For replication, a 552-patient cohort of tumors arising in men of European descent was compiled from The Cancer Genome Atlas (TCGA)²⁹ supplemented by 140 primary prostate cancers with blood and tumor whole genome sequencing²⁶. The replication cohort was analyzed identically to the discovery cohort (see Methods below). Finally, to assess the dQTL generalizability, cancer types in the Pan-Cancer Analysis of Whole Genomes (PCAWG) cohort with ≥90 individuals of European descent (i.e. breast, ovarian, pancreatic) were analyzed.

As a positive control, two previously reported SNP associations with TMPRSS2-ERG fusion (T2E) were replicated, rs16901979 (OR=0.50; P=3.90×10⁻²; FIG. 7C) and rs1859962 (OR=1.52; P=5.05×10⁻³; FIG. 7D)³⁰. From a clinical perspective, two SNPs in HSD3B1 associated with overall survival in advanced prostate cancer³¹ showed trend associations with clinical relapse (Pᵣₛ₁₈₅₆₈₈₈=0.11; Pᵣₛ₁₀₄₇₃₀₃=0.18; FIGS. 7E-7F) and tumor extent at diagnosis (Pᵣₛ₁₈₅₆₈₈₈=0.029; Pᵣₛ₁₀₄₇₃₀₃=0.091; FIGS. 7G-7H). Reported associations of SNPs with PTEN loss¹⁷ and SPOP point mutations were not replicated in this cohort¹⁸. Finally, the inventors discovered an association between SNPs in the APOE gene and metastasis-free survival, extending an observation in melanoma (P=0.027; FIG. 7I)³²: tumors with the APOE2 genotype had a significantly higher GR burden (OR=0.45; P=0.05; FIG. 7J). These positive controls confirm the inventors' patient cohorts replicate known germline-somatic associations, but highlight the potential for false negatives at this level of statistical power.

Germline Risk Inversely Associated with Somatic Mutation Burden

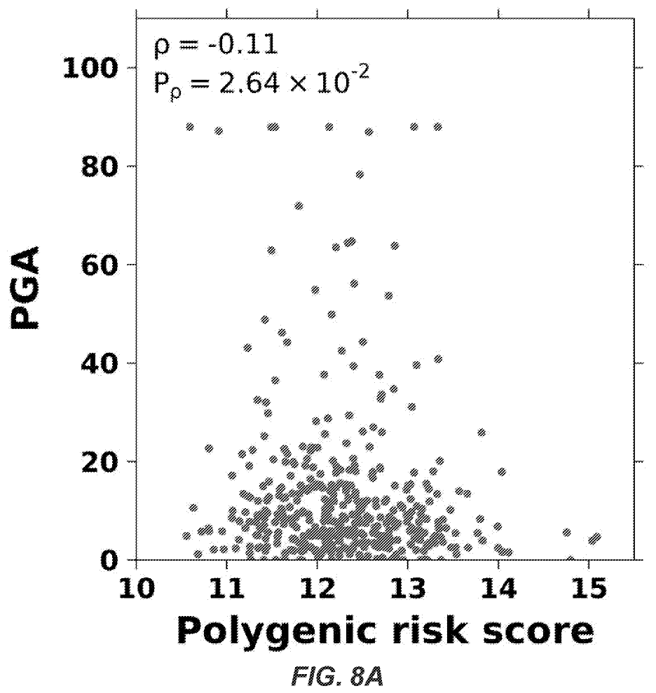

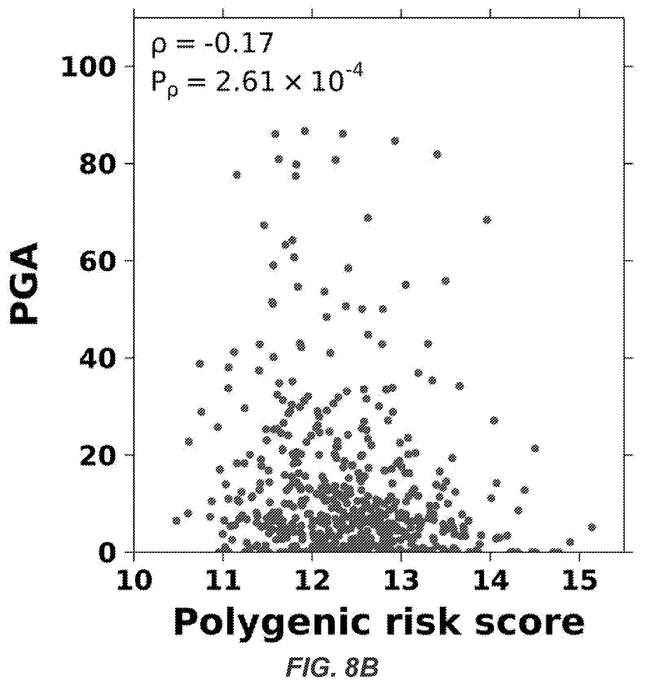

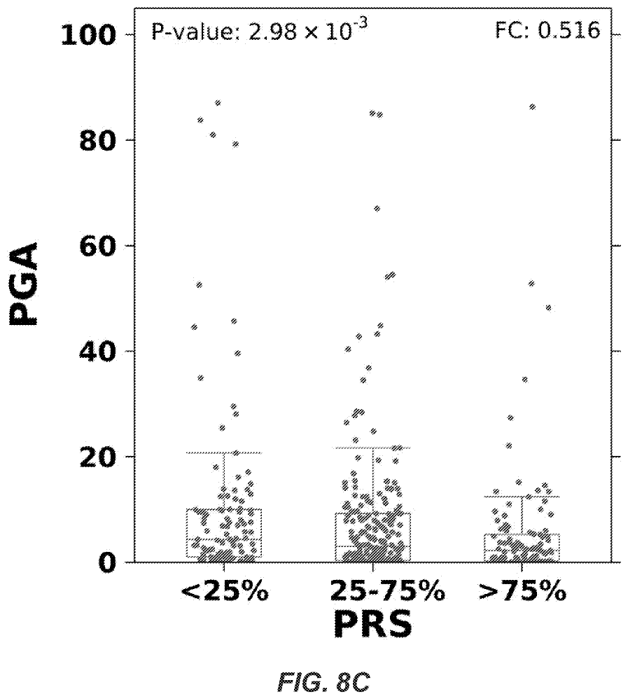

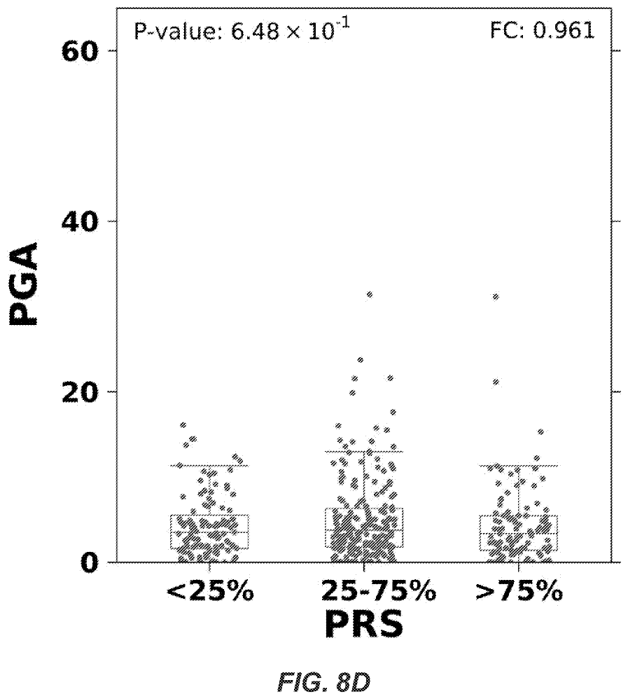

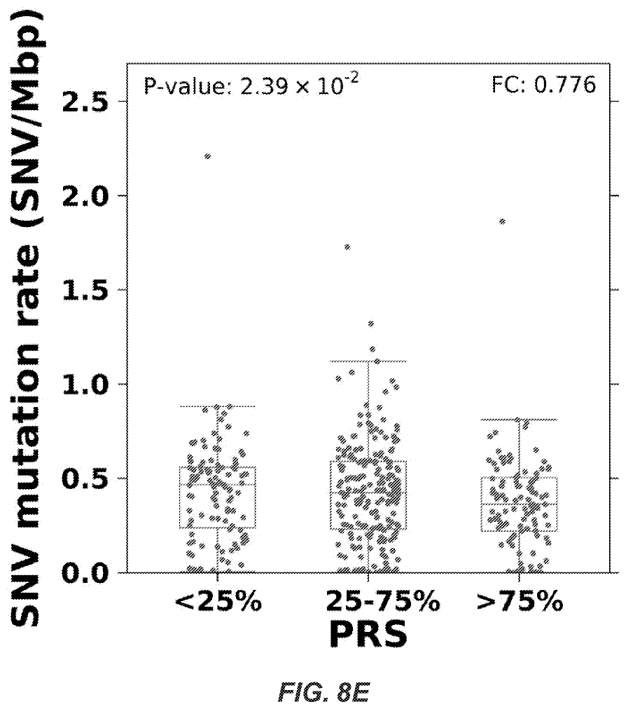

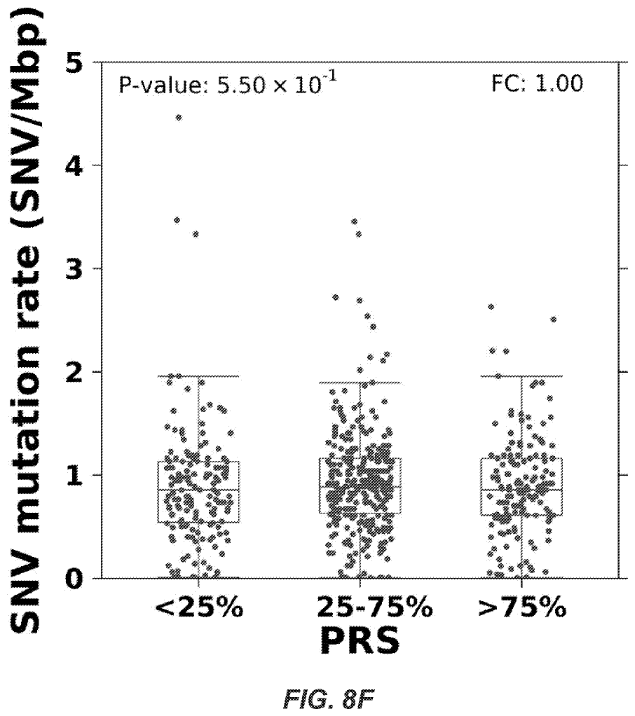

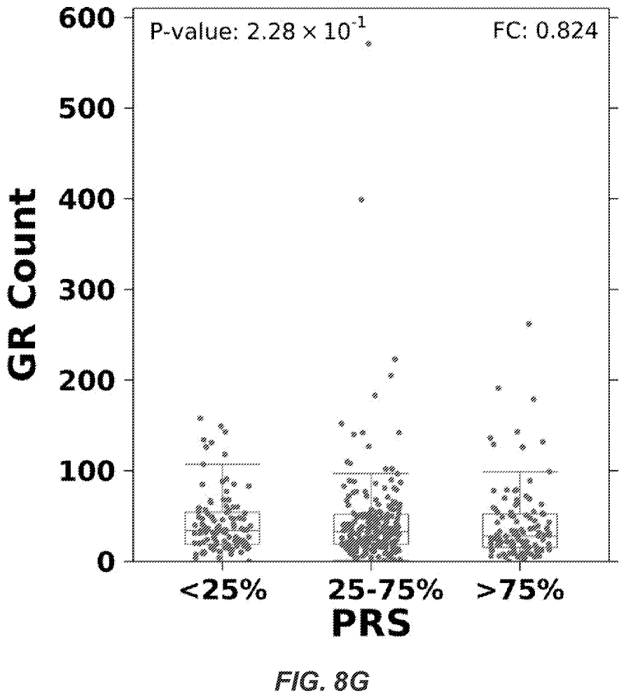

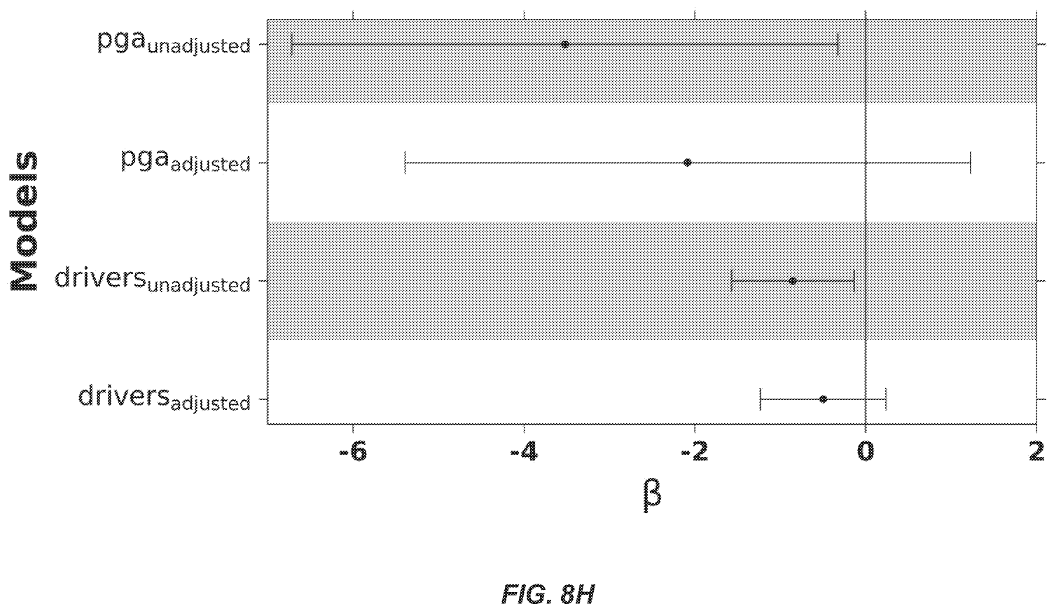

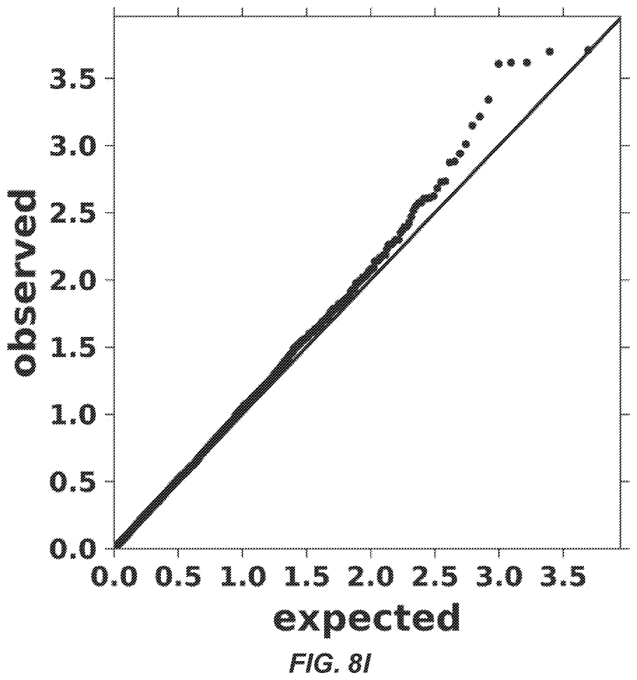

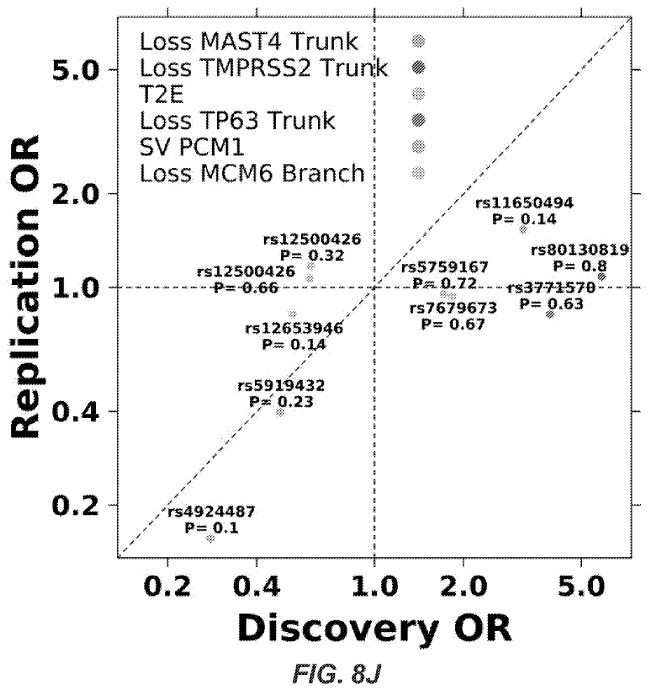

To identify germline SNPs associated with somatic drivers, the inventors considered a prostate cancer polygenic risk score (PRS) derived from 147 variants⁹. This aggregated measure of genetic risk was not significantly associated with any individual somatic driver (FDR>0.1; Table 2). Instead, genetic risk was significantly inversely associated with tumor ploidy, as measured by the proportion of the genome copy number altered (PGA). This relationship was consistent in both the discovery and replication cohorts (Fold Change between >75% and <25% (FC_{discovery})=0.71; P=4.46×10⁻³; FC_{replication}=0.49; P_{replication}=6.80×10⁻⁶; Mann-Whitney test; FIGS. 1B-1C; FIGS. 8A-8B). This association was stronger for subclonal (FC=0.52; P=2.98×10⁻³; FIG. 8C) than clonal CNAs (FC=0.96; P=0.65; FIG. 8D). Somatic point mutation burden was also inversely correlated with genetic risk, but this association could not be replicated in the exome-sequencing data comprising most of the inventors' replication cohort (FC_{SNV|discovery}=0.78; P_{SNV|discovery}=2.39×10⁻²; FIGS. 8E-8F). GR counts were not associated with genetic risk (FIG. 8G).

Tumors arising in individuals with higher genetic risk showed fewer somatic driver mutations (FC_{discovery}=0.80; P_{discovery}=3.65×10⁻²; FC_{replication}=0.83; P_{replication}=1.13×10⁻²; FIGS. 1D-1E). The association between PRS and somatic mutation burden may be mediated at least in part by patients with higher genetic risk being diagnosed at younger ages (FIG. 8H). This suggests baseline germline risk influences the number of somatic alterations required for tumorigenesis³³, a polygenic analog to Knudsen's “two-hit” hypothesis of retinoblastoma²¹.

Of the 134 individual risk SNPs with a minor allele frequency (MAF)>0.05 in the discovery cohort, ten were associated with one or more somatic driver mutations (logistic regression; FDR<0.1; FIG. 1F; FIG. 8I; Table 2 and Table 3). rs12500426 was associated with both loss of TMPRSS2 and the gene fusion between TMPRSS2 and ERG (OR=0.60 & 0.61; FDR=0.093 & 0.048, respectively; FIG. 1F). The inventors replicated previous reports of rs7679673 (OR=1.83; FDR=0.032) and rs12653946 (OR=0.53; FDR=0.032) association with ERG status³³ in the inventors' discovery cohort (FIG. 8J).

TABLE 2
Summary statistics from PRS associated with somatic drivers. β
and p-value from logistic regression correcting for two genetic principal
components and somatic mutation burden. FDR = false discovery rate.

| Driver | P value | Beta | FDR |
|---|---|---|---|
| T2E (TMPRSS2-ERG fusion) | 0.200136094 | −0.176754462 | 0.569618115 |
| Simple somatic mutation - | 0.91736276 | −0.019741223 | 0.969783489 |
| chr14.38037521.38073317 (Enhancer) | | | |
| Simple somatic mutation - FOXA1 (UTR) | 0.193057915 | −0.441989655 | 0.569618115 |
| Simple somatic mutation -SPOP (CDS) | 0.773212115 | 0.084833389 | 0.868589724 |
| Structural Variant - ERG | 0.15072739 | −0.259736515 | 0.569618115 |
| Structural Variant - KCNH7 | 0.731830511 | −0.114593745 | 0.868589724 |
| Structural Variant - PCM1 | 0.266366847 | 0.322069224 | 0.615973333 |
| Structural Variant - PTEN | 0.372289002 | 0.196931849 | 0.724983847 |
| Structural Variant - TMPRSS2 | 0.999743938 | 5.24885E−05 | 0.999743938 |
| Structural Variant - TTC28 | 0.411114477 | −0.22143473 | 0.747093225 |
| Reduced expression and/or deletion - | 0.262738932 | 0.271023194 | 0.615973333 |
| ARID4B (Trunk) | | | |
| Reduced expression and/or deletion - | 0.130600627 | 0.3294092 | 0.569618115 |
| MCM6 (Trunk) | | | |
| Reduced expression and/or deletion - | 0.023888096 | −0.44199963 | 0.294619851 |
| PPP4R2 (Trunk) | | | |
| Reduced expression and/or deletion - | 0.263445169 | 0.327164149 | 0.615973333 |
| TP63 (Trunk) | | | |
| Reduced expression and/or deletion - | 0.524984428 | 0.144244718 | 0.747093225 |
| MAST4 (Trunk) | | | |
| Reduced expression and/or deletion - | 0.01209424 | 0.508688971 | 0.294619851 |
| PPIP5K2 (Trunk) | | | |
| Reduced expression and/or deletion - | 0.158709208 | 0.217030023 | 0.569618115 |
| ZNF292 (Trunk) | | | |
| Reduced expression and/or deletion - | 0.14874877 | −0.197980807 | 0.569618115 |
| NKX3.1 (Trunk) | | | |
| Reduced expression and/or deletion - | 0.70435464 | −0.064575487 | 0.868589724 |
| PTEN (Trunk) | | | |
| Reduced expression and/or deletion - | 0.524497771 | −0.19820191 | 0.747093225 |
| ATM (Trunk) | | | |
| Reduced expression and/or deletion - | 0.113183767 | −0.241968208 | 0.569618115 |
| RB1 (Trunk) | | | |
| Reduced expression and/or deletion - | 0.971622295 | 0.005799175 | 0.998611803 |
| KLF5 (Trunk) | | | |
| Reduced expression and/or deletion - | 0.449644758 | −0.125655192 | 0.747093225 |
| FBXO31 (Trunk) | | | |
| Reduced expression and/or deletion - | 0.80596432 | −0.038095355 | 0.877078819 |
| TMPRSS2 (Trunk) | | | |
| Increased copy number - NCOA2 (Trunk) | 0.607522119 | −0.120464317 | 0.832530311 |
| Increased copy number - MYC (Trunk) | 0.369386223 | 0.198121843 | 0.724983847 |
| Reduced expression and/or deletion - | 0.091703315 | −0.657985525 | 0.569618115 |
| MCM6 (Branch) | | | |
| Reduced expression and/or deletion - | 0.192895484 | −0.350411713 | 0.569618115 |
| MAST4 (Branch) | | | |
| Reduced expression and/or deletion - | 0.715802418 | 0.087595977 | 0.868589724 |
| CHD1 (Branch) | | | |
| Reduced expression and/or deletion - | 0.122080219 | −0.355765443 | 0.569618115 |
| ZNF292 (Branch) | | | |
| Reduced expression and/or deletion - | 0.511052058 | 0.119695307 | 0.747093225 |
| RNLS (Branch) | | | |
| Reduced expression and/or deletion - | 0.774688132 | −0.064469585 | 0.868589724 |
| CDKN1B (Branch) | | | |
| Reduced expression and/or deletion - | 0.019618587 | −0.653985363 | 0.294619851 |
| RAN (Branch) | | | |
| Reduced expression and/or deletion - | 0.70213195 | −0.076219125 | 0.868589724 |
| RB1 (Branch) | | | |
| Reduced expression and/or deletion - | 0.487764982 | −0.135781827 | 0.747093225 |
| FBXO31 (Branch) | | | |
| Reduced expression and/or deletion - | 0.315613957 | 0.230922652 | 0.686924494 |
| HMSD (Branch) | | | |
| Reduced expression and/or deletion - | 0.454047718 | −0.201846154 | 0.747093225 |
| NCOA2 (Branch) | | | |

TABLE 3
Number of dQTLs identified for each somatic driver in each analysis strategy.

| Driver | Trans | Spatial | Enhancer | Linear |
|---|---|---|---|---|
| T2E (TMPRSS2-ERG fusion) | 2 | 0 | 1 | 1 |
| Simple somatic mutation - FOXA1 UTR | 0 | 0 | 3 | 0 |
| Structural Variant - PCM1 | 0 | 0 | 0 | 0 |
| Structural Variant - TTC28 | 1 | 0 | 1 | 0 |
| Reduced expression and/or deletion - | 1 | 0 | 0 | 0 |
| ARID4B (Trunk) | | | | |
| Reduced expression and/or deletion - | 0 | 0 | 1 | 1 |
| PPP4R2 (Trunk) | | | | |
| Reduced expression and/or deletion - | 0 | 0 | 0 | 0 |
| TP63 (Trunk) | | | | |
| Reduced expression and/or deletion - | 2 | 0 | 0 | 0 |
| MAST4 (Trunk) | | | | |
| Reduced expression and/or deletion - | 3 | 0 | 0 | 2 |
| ZNF292 (Trunk) | | | | |
| Reduced expression and/or deletion - | 0 | 2 | 1 | 0 |
| RB1 (Trunk) | | | | |
| Reduced expression and/or deletion - | 0 | 0 | 4 | 0 |
| FBXO31 (Trunk) | | | | |
| Reduced expression and/or deletion - | 1 | 0 | 0 | 2 |
| TMPRSS2 (Trunk) | | | | |
| Increased copy number - NCOA2 | 2 | 0 | 0 | 1 |
| (Trunk) | | | | |
| Increased copy number - MYC | 1 | 0 | 0 | 2 |
| (Trunk) | | | | |
| Reduced expression and/or deletion - | 3 | 0 | 0 | 0 |
| MCM6 (Branch) | | | | |
| Reduced expression and/or deletion - | 0 | 0 | 0 | 0 |
| MAST4 (Branch) | | | | |
| Reduced expression and/or deletion - | 0 | 0 | 1 | 0 |
| CHD1 (Branch) | | | | |
| Reduced expression and/or deletion - | 2 | 0 | 1 | 3 |
| CDKN1B (Branch) | | | | |
| Reduced expression and/or deletion - | 0 | 2 | 0 | 0 |
| RAN (Branch) | | | | |
| Increased copy number - NCOA2 | 0 | 0 | 0 | 4 |
| (Branch) | | | | |
| Simple somatic mutation - chr14 | 0 | 0 | 0 | 0 |
| 38037521 38073317 Enhancer | | | | |
| Simple somatic mutation - SPOP CDS | 0 | 0 | 0 | 0 |
| Structural Variant - ERG | 0 | 0 | 0 | 0 |
| Structural Variant - KCNH7 | 0 | 0 | 0 | 0 |
| Structural Variant - PTEN | 0 | 0 | 0 | 0 |
| Structural Variant - TMPRSS2 | 0 | 0 | 0 | 0 |
| Reduced expression and/or deletion - | 0 | 0 | 0 | 0 |
| MCM6 (Trunk) | | | | |
| Reduced expression and/or deletion - | 0 | 0 | 0 | 0 |
| PPIP5K2 (Trunk) | | | | |
| Reduced expression and/or deletion - | 0 | 0 | 0 | 0 |
| NKX3 1 (Trunk) | | | | |
| Reduced expression and/or deletion - | 0 | 0 | 0 | 0 |
| PTEN (Trunk) | | | | |
| Reduced expression and/or deletion - | 0 | 0 | 0 | 0 |
| ATM (Trunk) | | | | |
| Reduced expression and/or deletion - | 0 | 0 | 0 | 0 |
| KLF5 (Trunk) | | | | |
| Reduced expression and/or deletion - | 0 | 0 | 0 | 0 |
| ZNF292 (Branch) | | | | |
| Reduced expression and/or deletion - | 0 | 0 | 0 | 0 |
| RNLS (Branch) | | | | |
| Reduced expression and/or deletion - | 0 | 0 | 0 | 0 |
| RB1 (Branch) | | | | |
| Reduced expression and/or deletion - | 0 | 0 | 0 | 0 |
| FBXO31 (Branch) | | | | |
| Reduced expression and/or deletion - | 0 | 0 | 0 | 0 |
| HMSD (Branch) | | | | |

Linear Local dQTLs Bias Somatic Drivers in Prostate Cancer

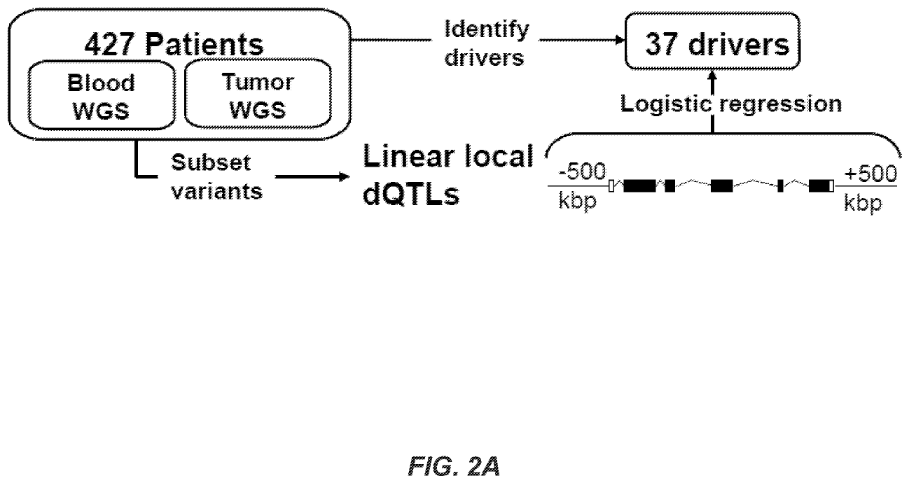

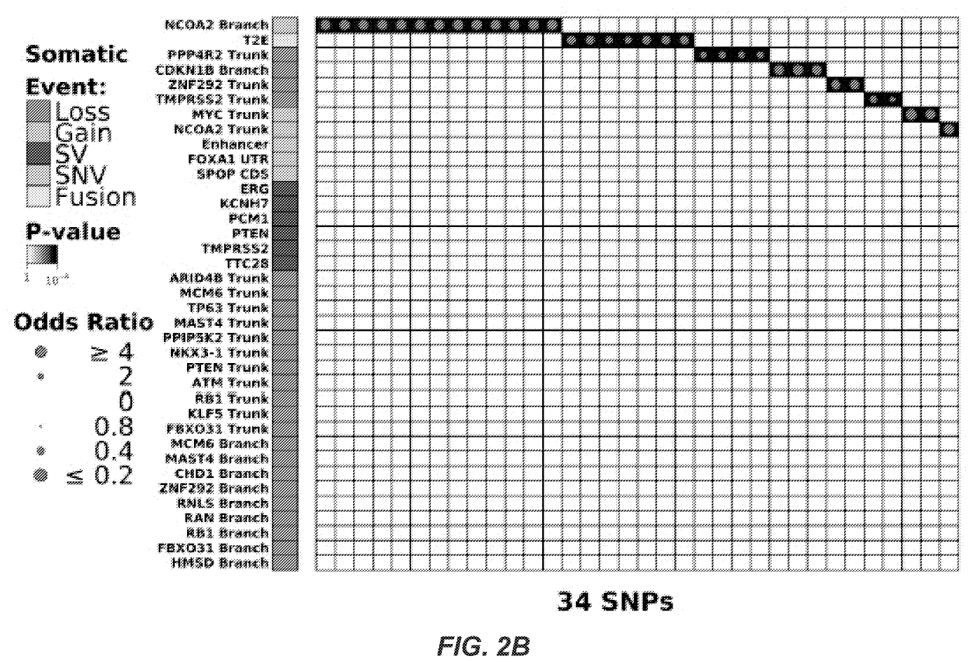

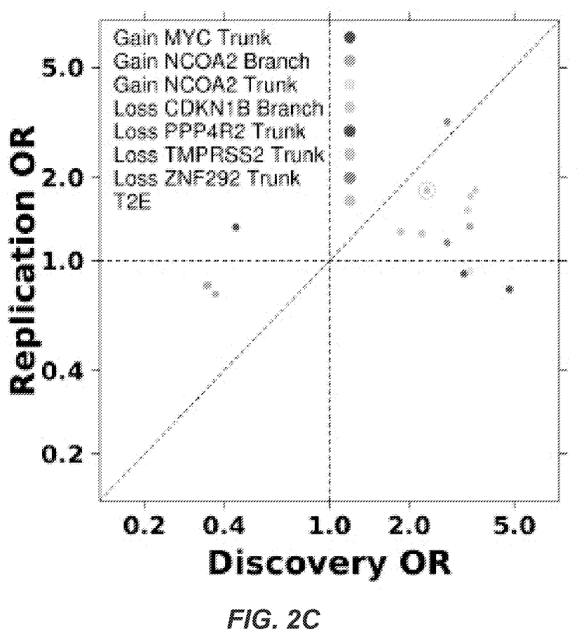

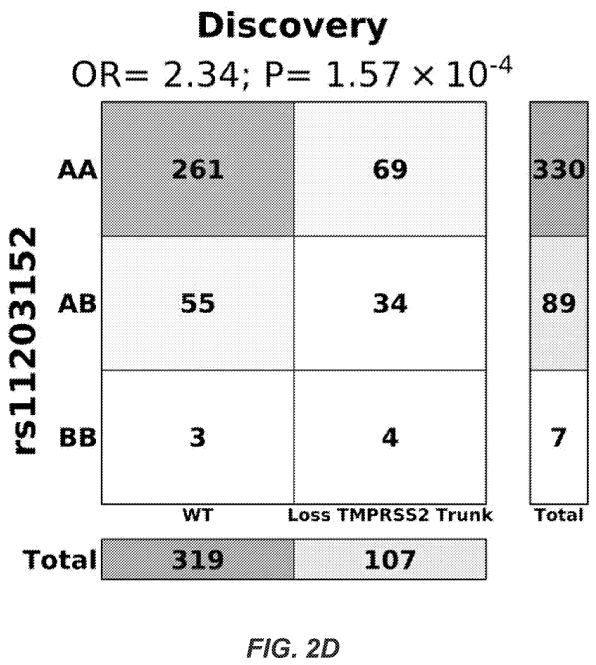

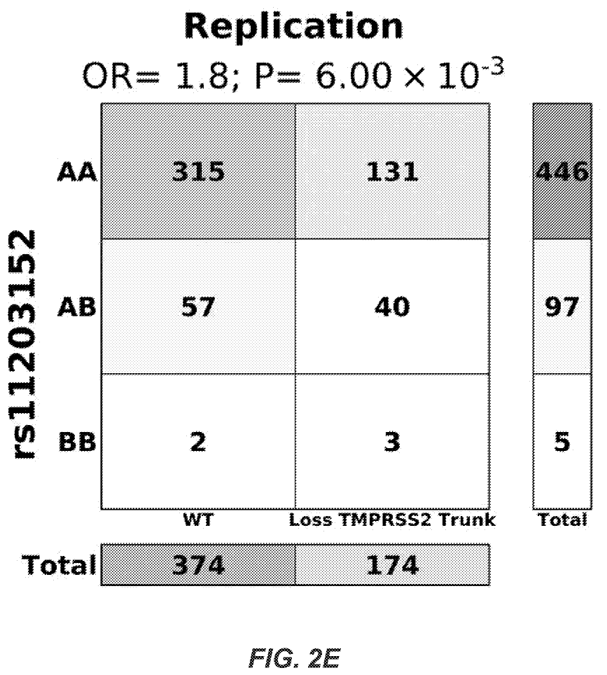

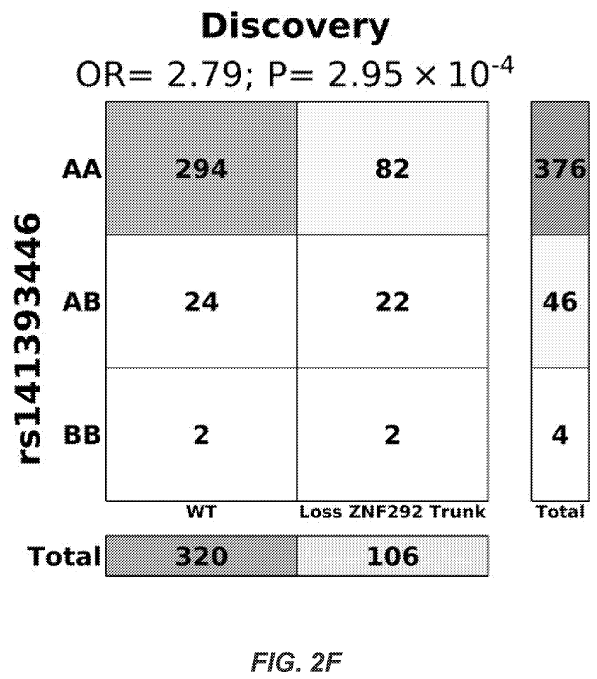

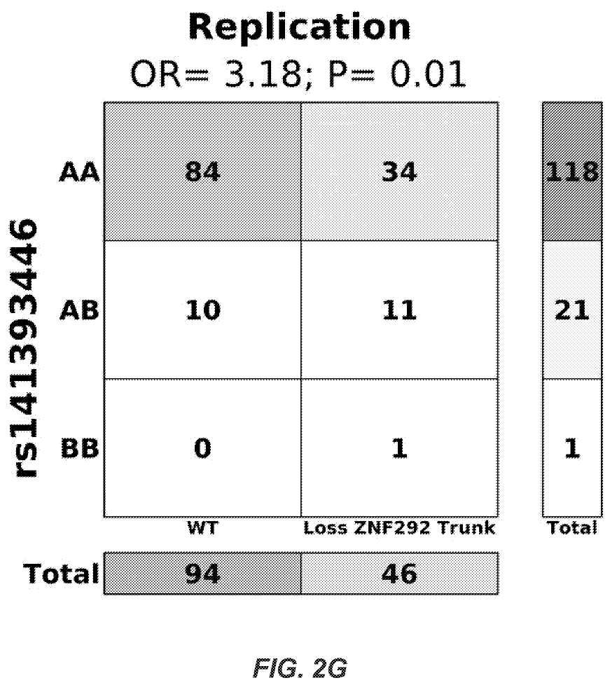

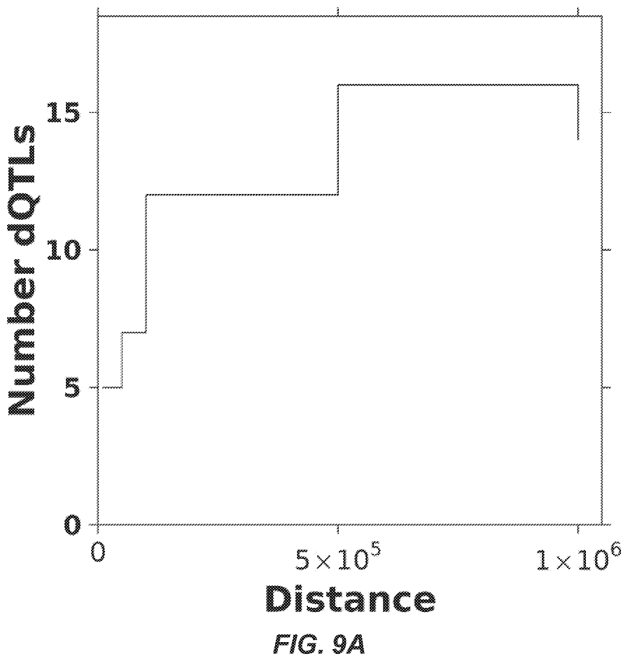

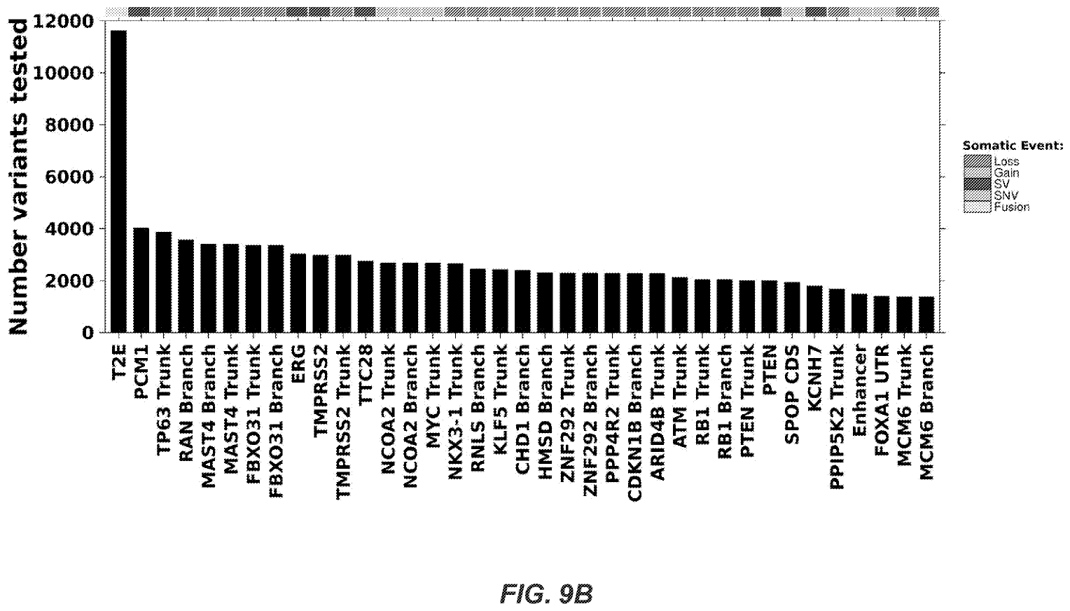

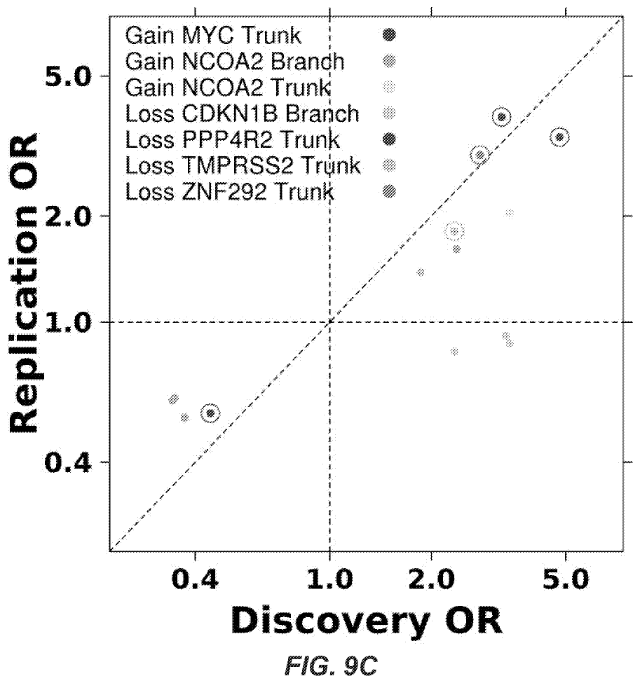

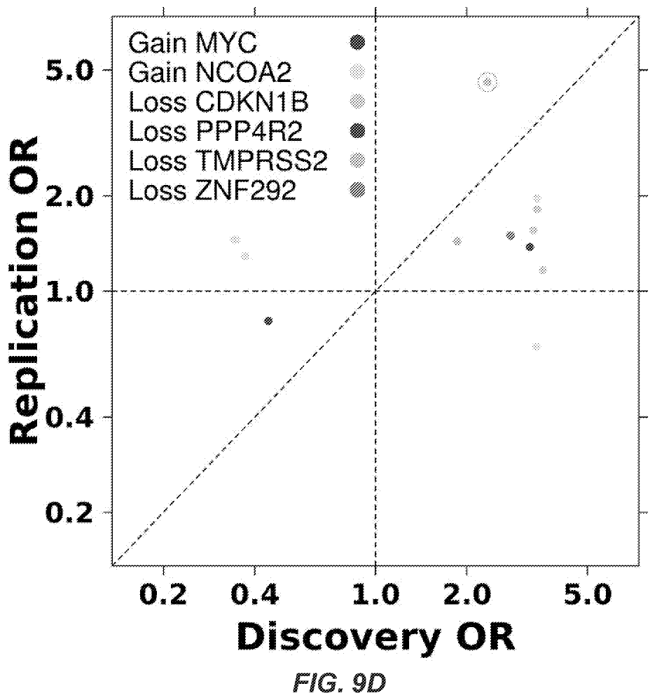

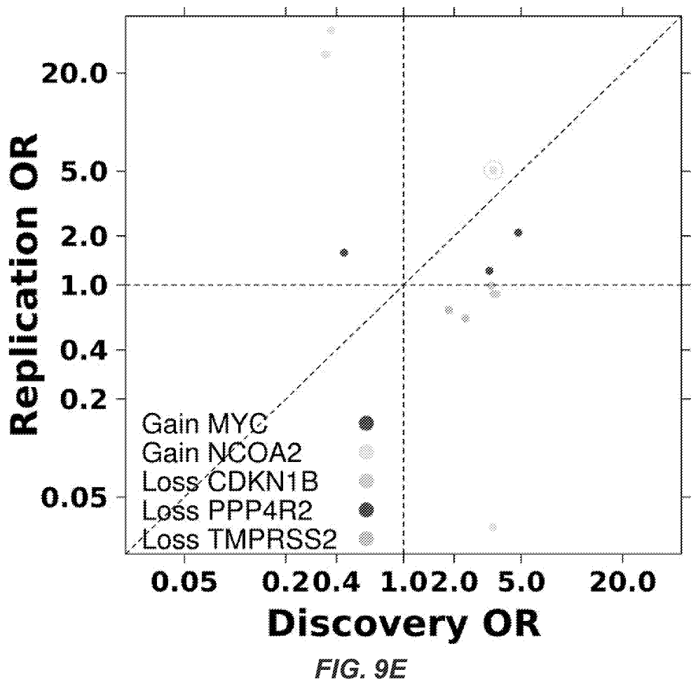

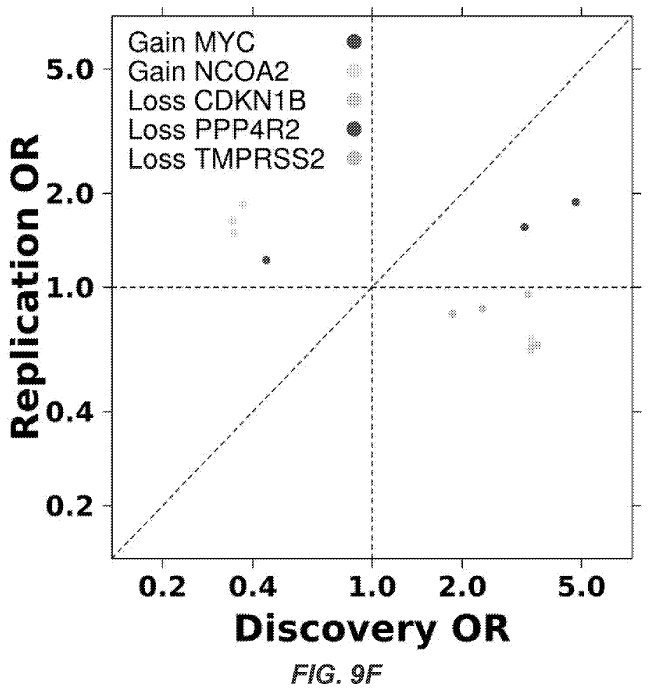

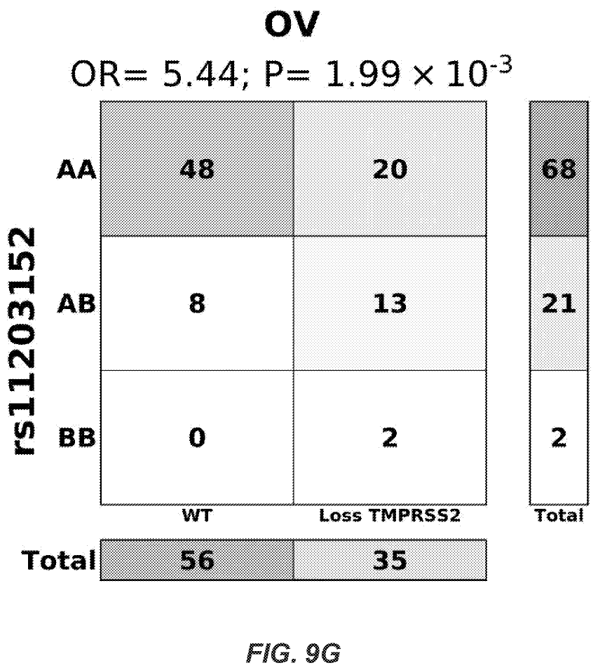

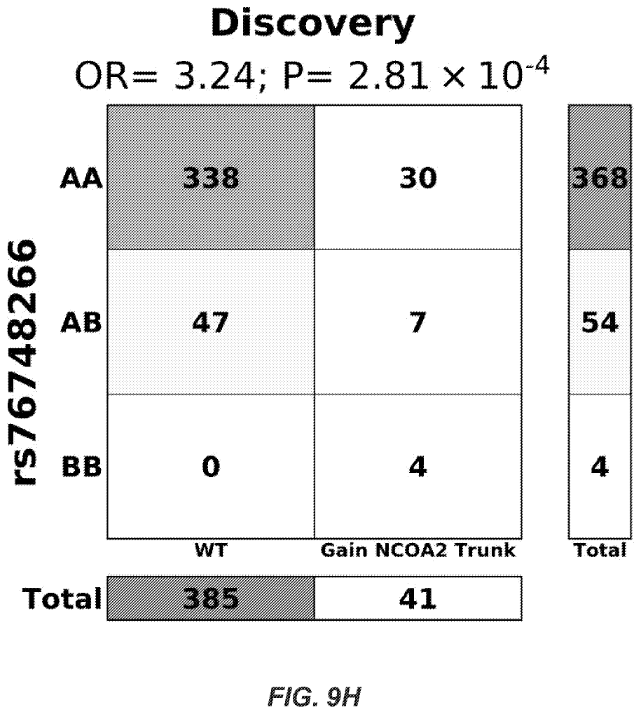

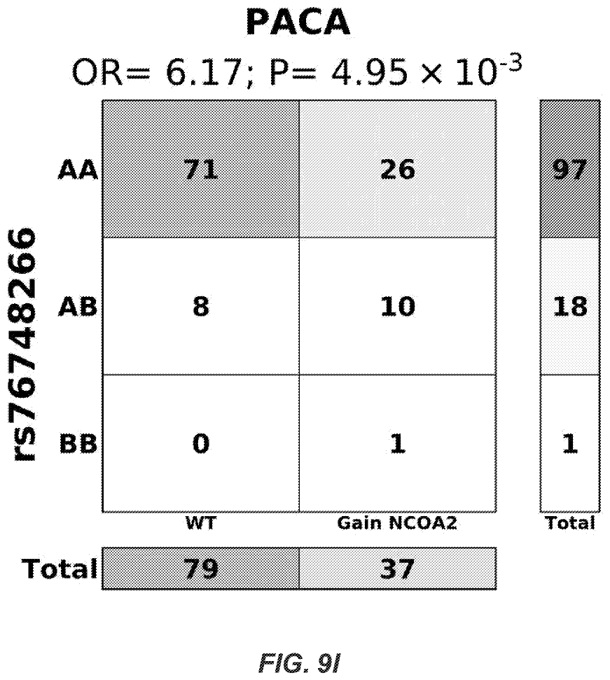

The association of PRS with somatic mutational burden suggested that specific dQTLs might play a role in guiding the mutational and evolutionary diversity of localized prostate cancer³⁵. The inventors evaluated common SNPs (MAF>0.05) in close proximity with somatic driver mutations, defined as being within ±500 kbp of the event boundaries (FIG. 2A) based on sensitivity analysis (FIG. 9A). The inventors associated 37 somatic drivers with 1,332-11,618 germline SNPs (median=2,398, haplotype blocks=80-1,379; FIG. 9B). After controlling for population structure and somatic mutation burden, 34 local dQTLs were identified in 16 haplotype blocks, involving eight drivers (logistic regression; Bonferroni a=0.1; P_{unadjusted}<3.60×10⁻⁴; OR>1.86; FIG. 2B; Table 3). The inventors selected a tag dQTL—i.e. one SNP to represent each haplotype block—based on minimum p-value. A subset of the patients in the inventors' discovery cohort (n=325/427) had additional CNA profiling using orthogonal array-based platforms, and 12/15 CNA tag SNPs were verified by this independent technology (FIG. 9C).

Of the 16 tag dQTLs in the replication cohort, 12 exhibited consistent effect-sizes in the replication cohort (FIG. 2C), and one replicated directly (rs11203152 with loss of TMPRSS2; FIGS. 2D-2E). Association of rs141393446 with loss of ZNF292 was nominally replicated despite only being genotyped in 140 replication cohort samples with WGS (FIGS. 2F-2G). Of the 16 tag dQTLs, 14 showed consistent effect-sizes in ovarian, breast or pancreatic cancers³⁶ (FIGS. 9D-9F). Most notably, the association of rs11203152 with loss of TMPRSS2 replicated in ovarian cancer (ORₒᵥₐᵣᵢₐₙ=5.43; FDRₒᵥₐᵣᵢₐₙ=2.78×10⁻²; FIG. 9G) and the association of rs76748266 with gain of NCOA2 replicated in pancreatic cancer (OR_{pancreatic}=6.17; FDR_{pancreatic}=1.85×10⁻²; FIG. 9H-9I). Thus local dQTLs appear to influence multiple cancer types.

Spatial Local dQTLs Bias Somatic Drivers in Prostate Cancer

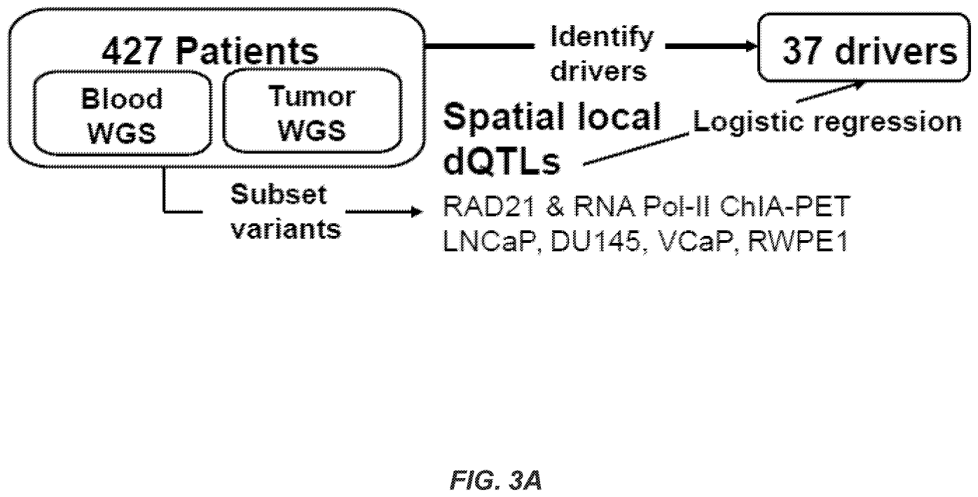

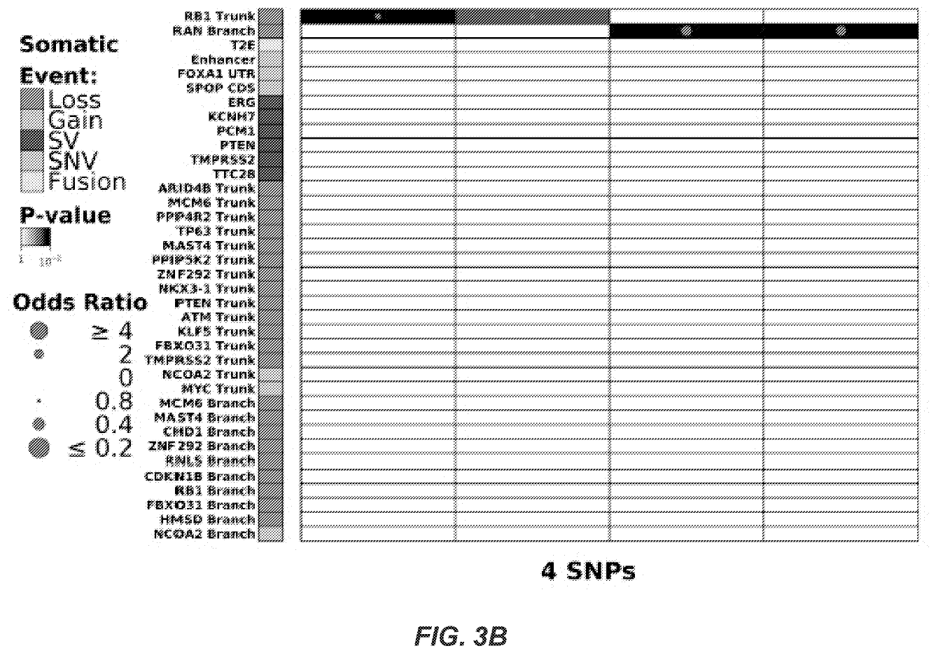

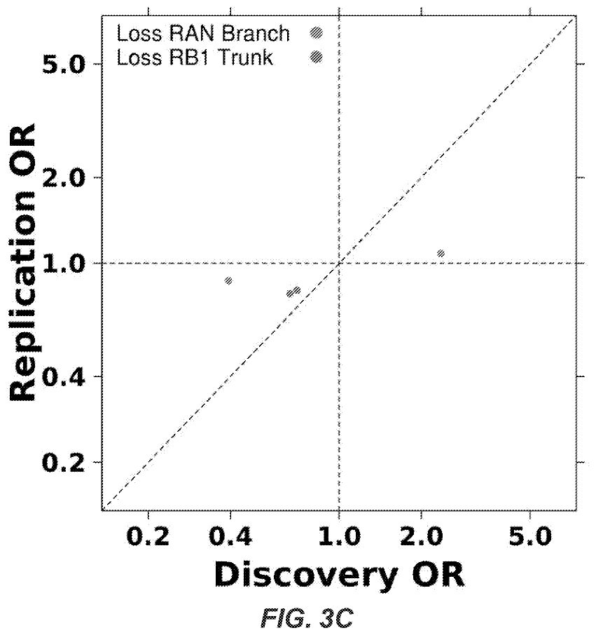

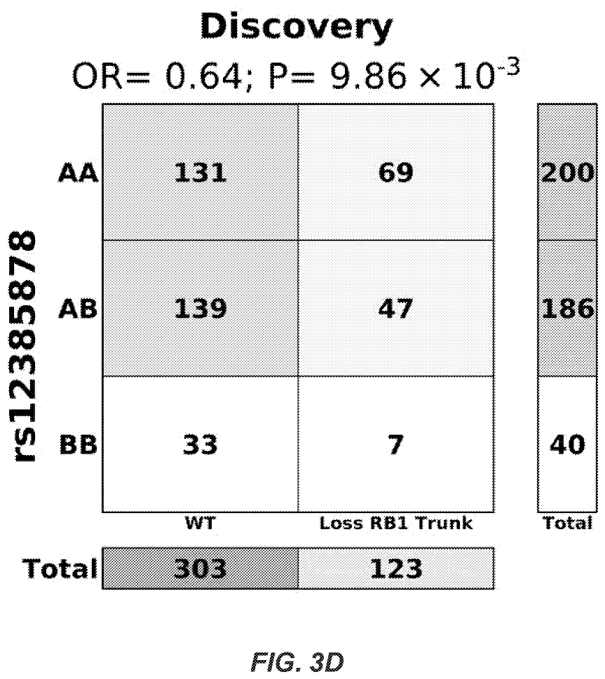

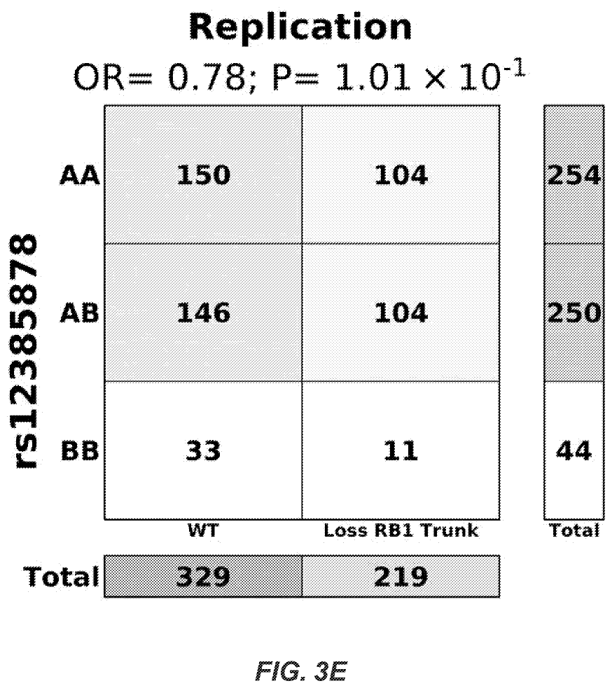

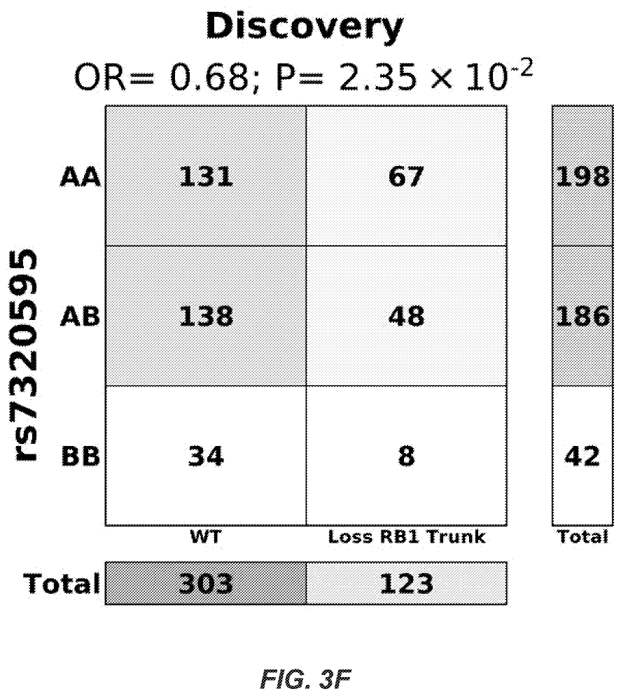

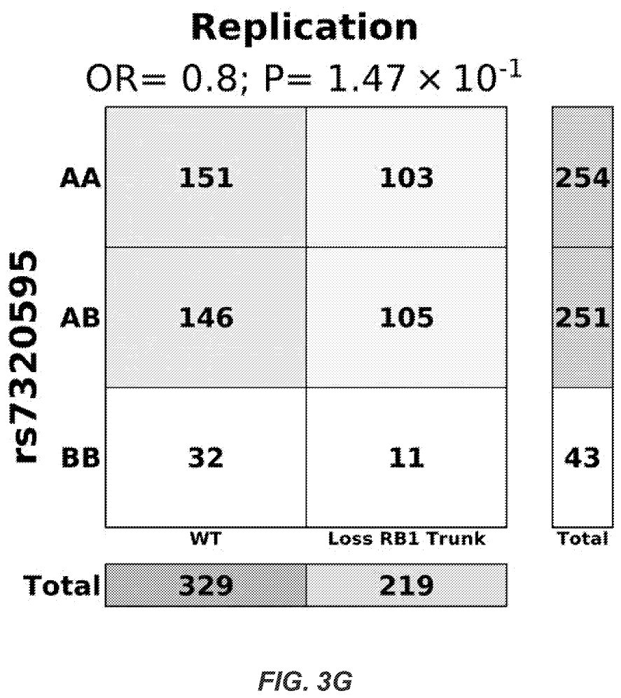

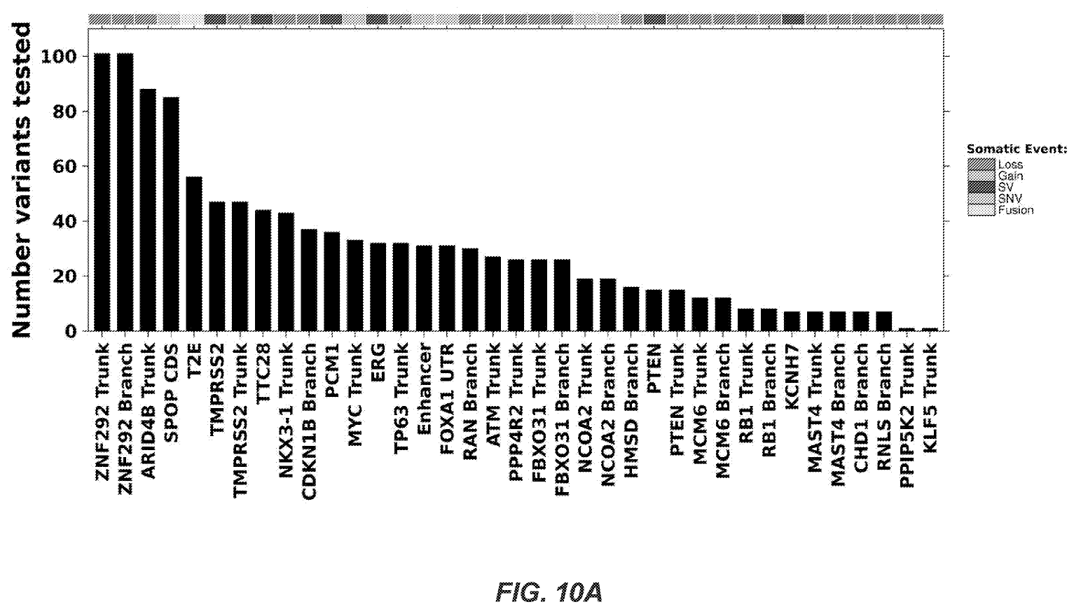

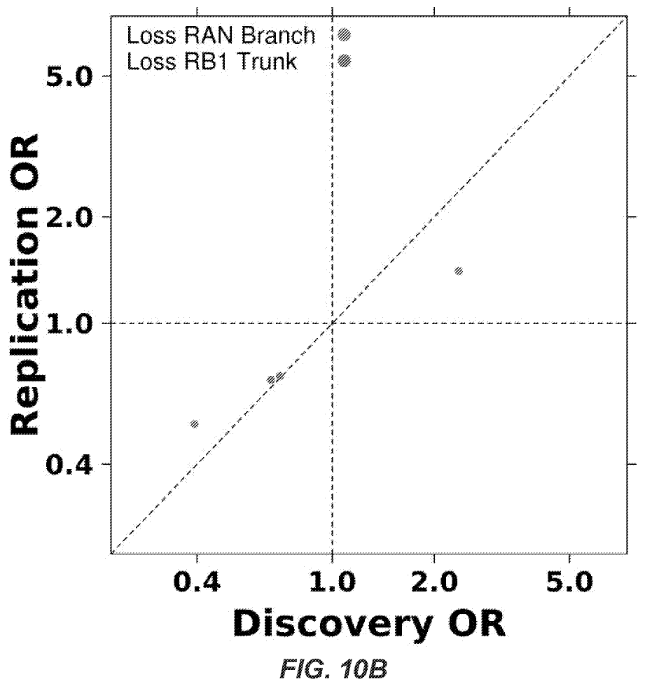

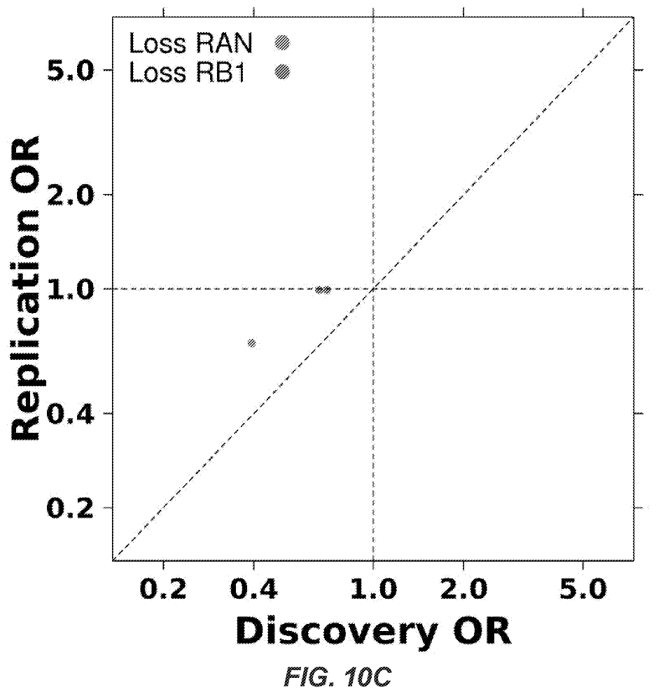

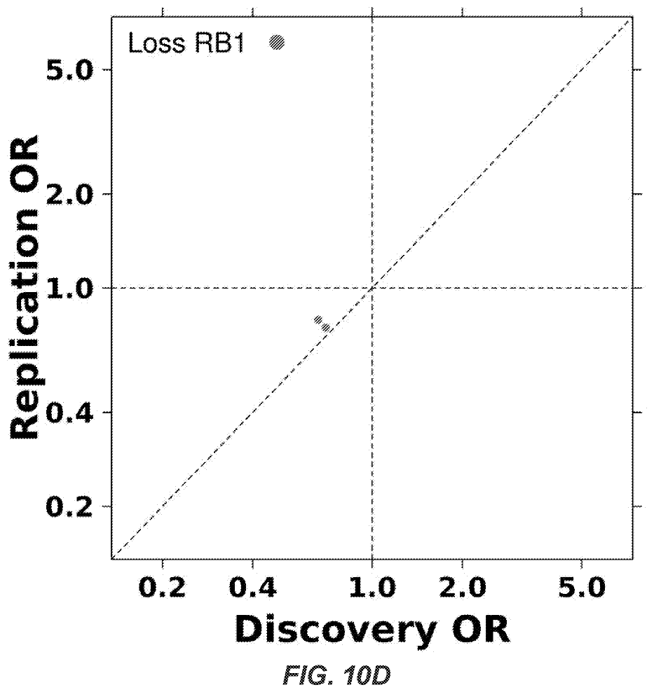

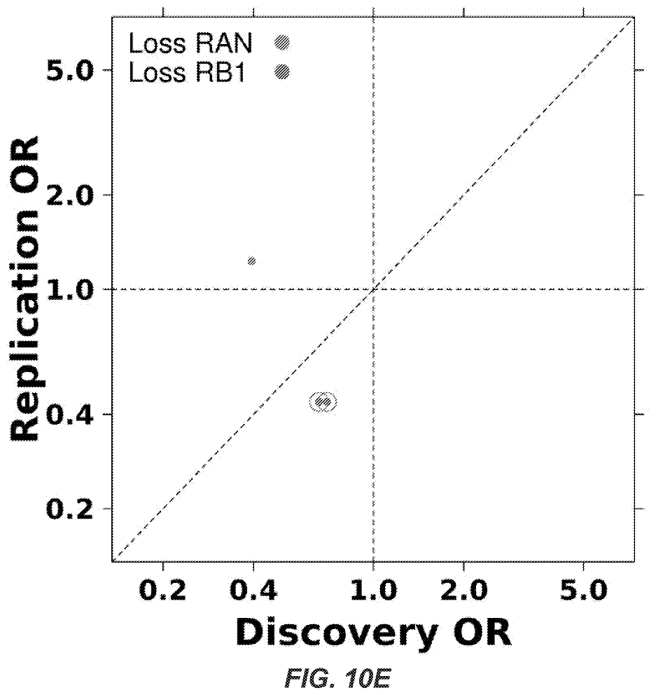

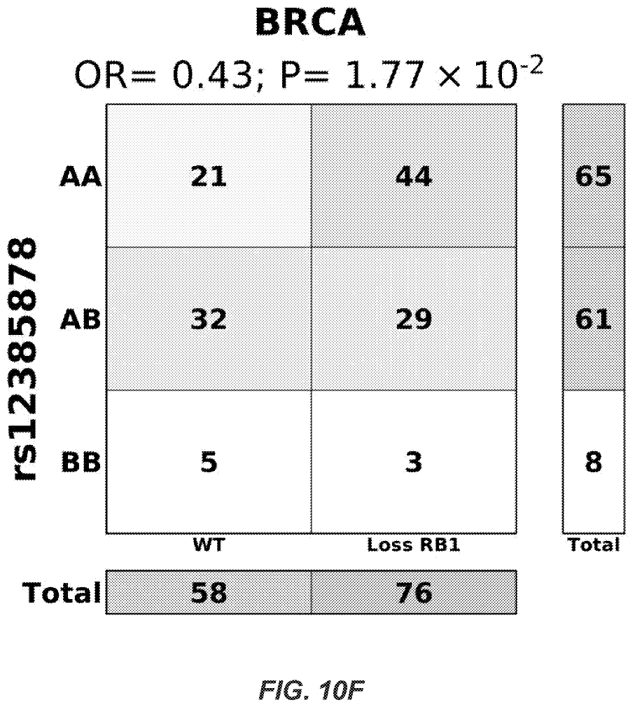

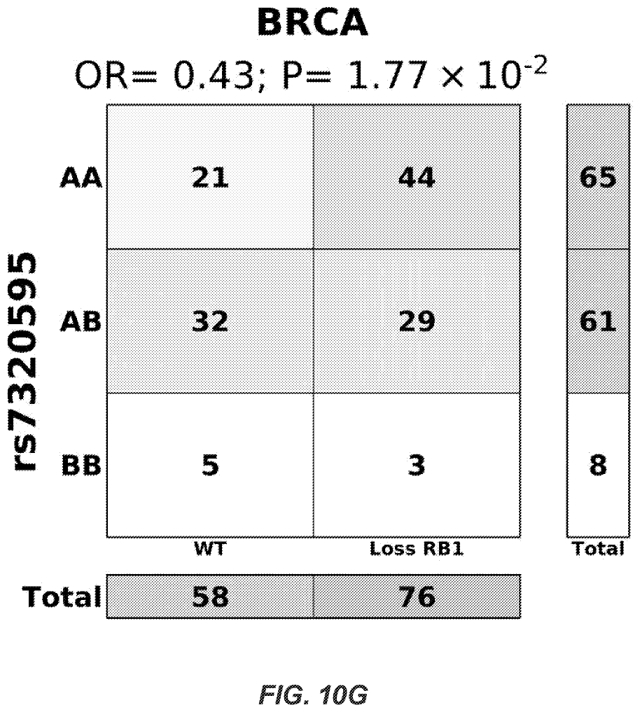

To expand the inventors' definition of local germline SNPs, the inventors considered SNPs in close proximity to the somatic event as defined by three-dimensional proximity based on DNA secondary structure. Spatial local dQTLs were defined based on three-dimensional proximity via RNA polymerase II and RAD21 ChIA-PET in LNCaP, DU145 and VCaP prostate cancer cells and RWPE-1 prostate epithelial cells³⁷ (FIG. 3A). The inventors identified regions outside the linear local boundaries that interacted with the event region in at least two of four cell lines (see Methods below). The 37 somatic drivers were evaluated for associations with 1-101 SNPs (median=26; haplotype blocks=1-20; FIG. 10A). The inventors discovered four dQTLs affecting two somatic drivers: subclonal loss of RAN and clonal loss of RB1 (logistic regression; Bonferroni α=0.1; P_{unadjusted}<2.35×10⁻²; OR>1.47; FIG. 3B; Table 3) All four spatial local dQTLs were verified using array-based CNA detection (FIG. 10B).

No spatial local dQTL reached global significance in the inventors' replication cohort (FIG. 3C). Two SNPs in strong linkage disequilibrium (r²=0.97) associated with loss of RB1 showed strong trend effects (FIGS. 3D-3G). All four spatial local dQTLs were tested in ovarian, pancreatic and breast cancer³⁶ (FIGS. 10C-10E). Both dQTLs associated with loss of RB1 replicated in breast cancer (FIGS. 10F-10G), showing spatial local dQTLs can exert pan-cancer effects and highlighting the value of pan-cancer studies in maximizing power for dQTL discovery.

Enhancer Local dQTLs Bias Somatic Drivers in Prostate Cancer

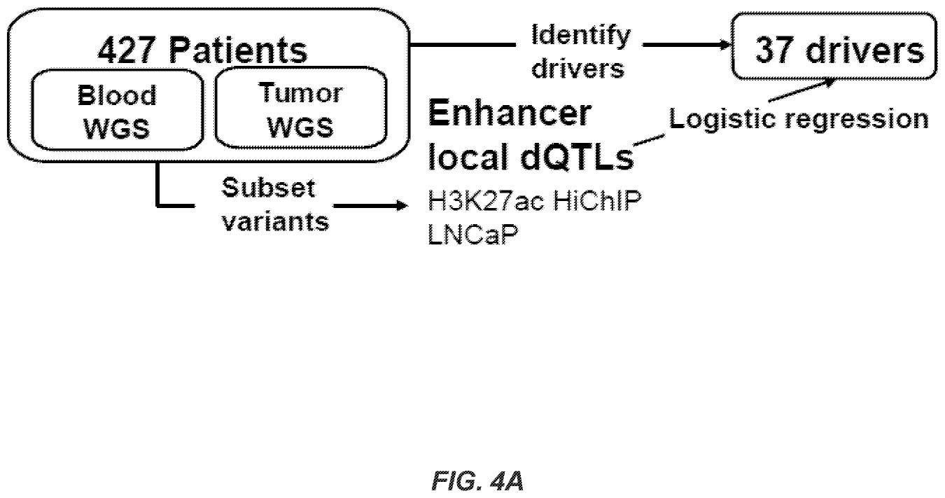

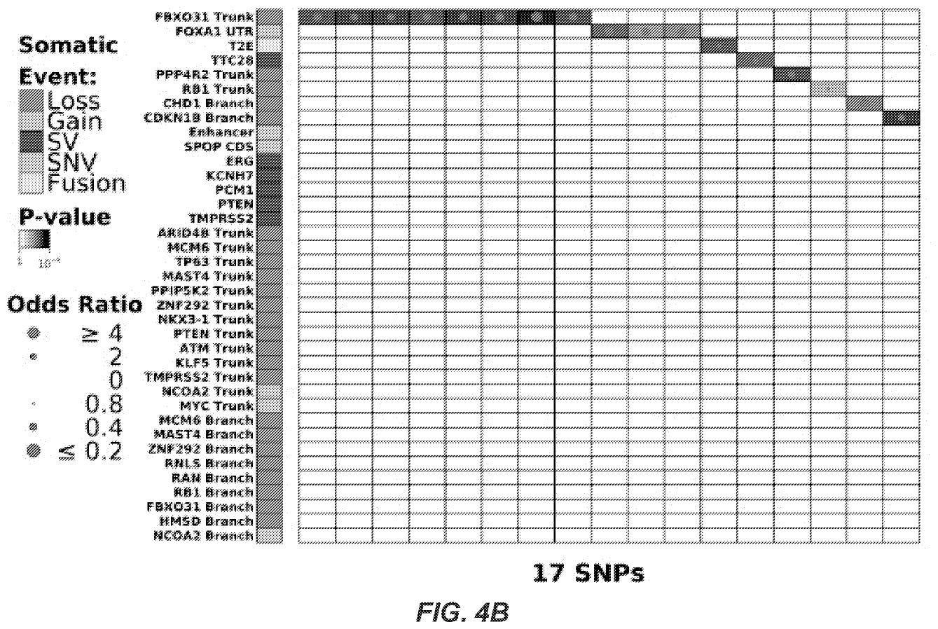

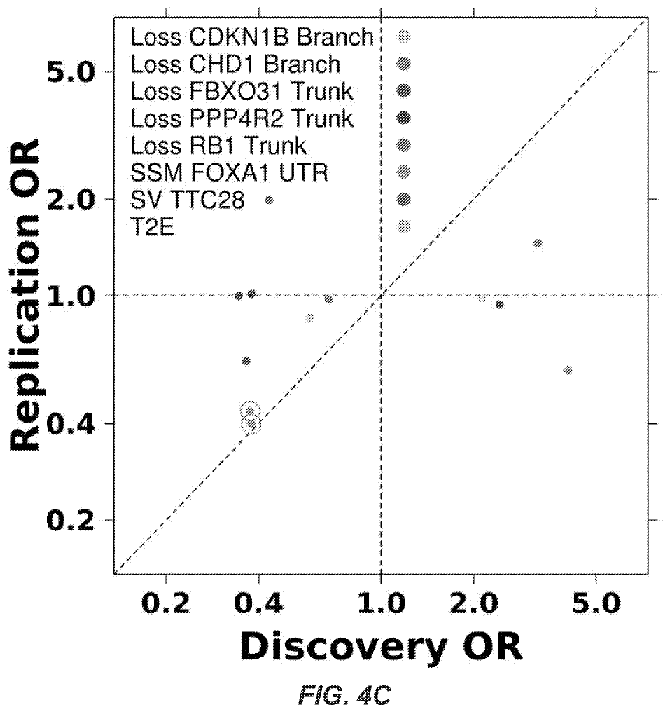

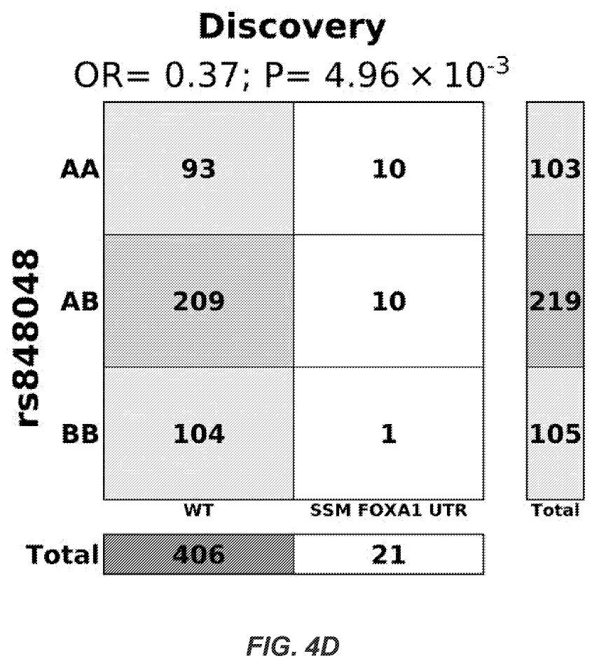

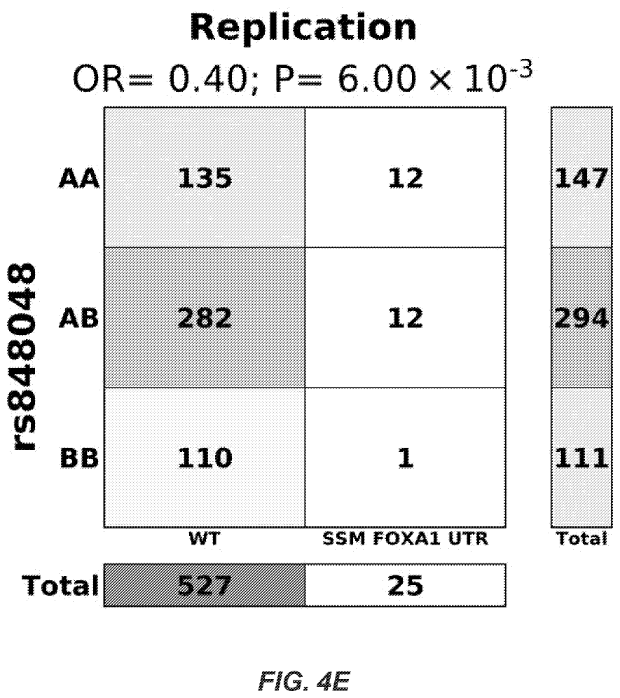

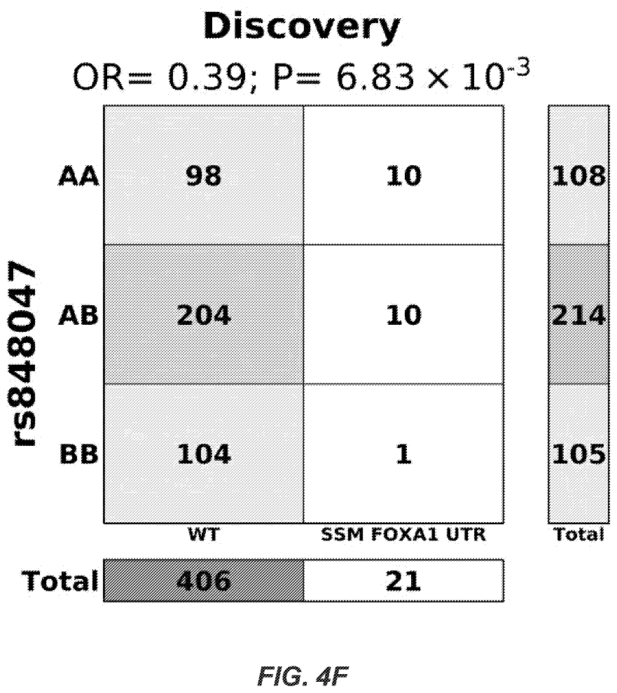

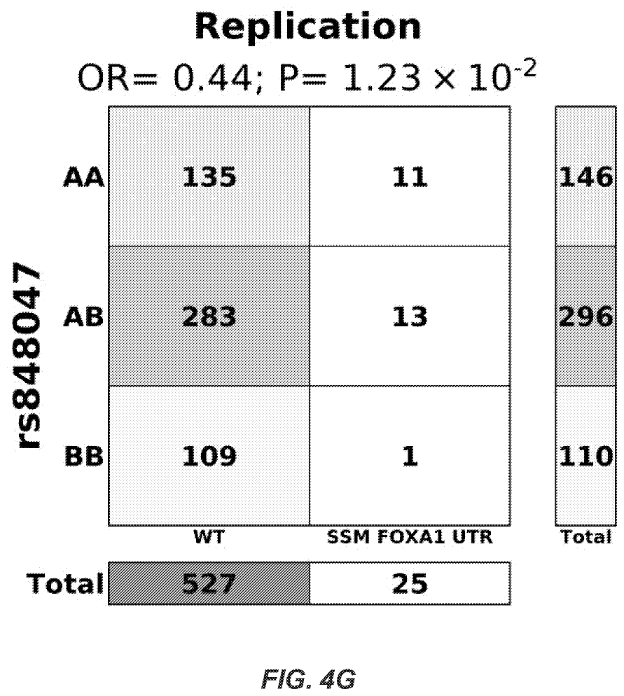

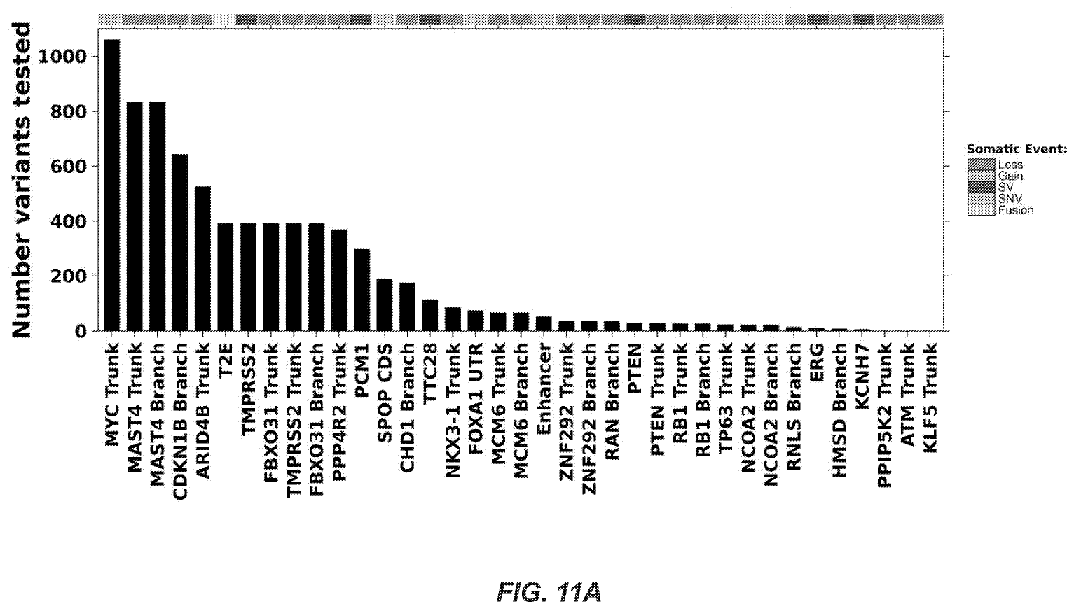

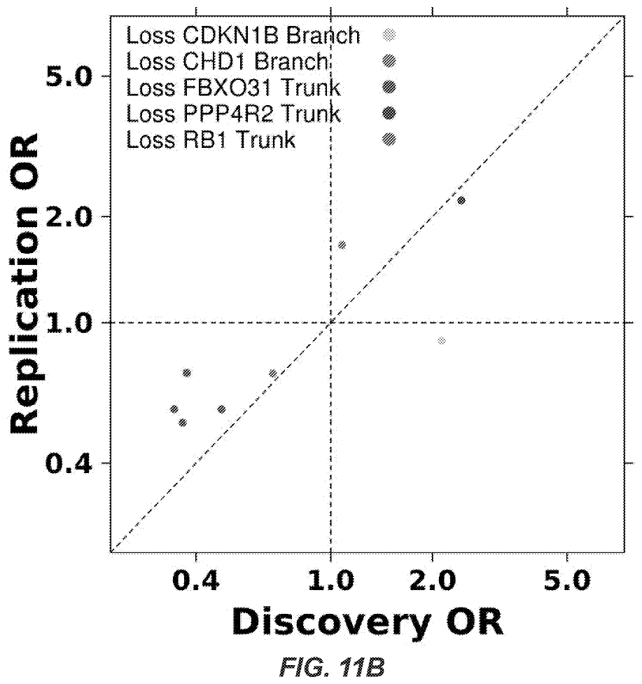

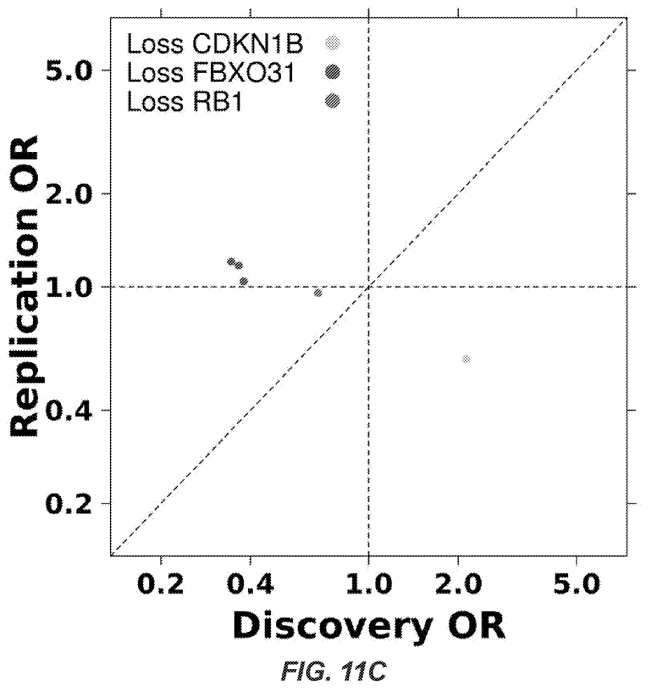

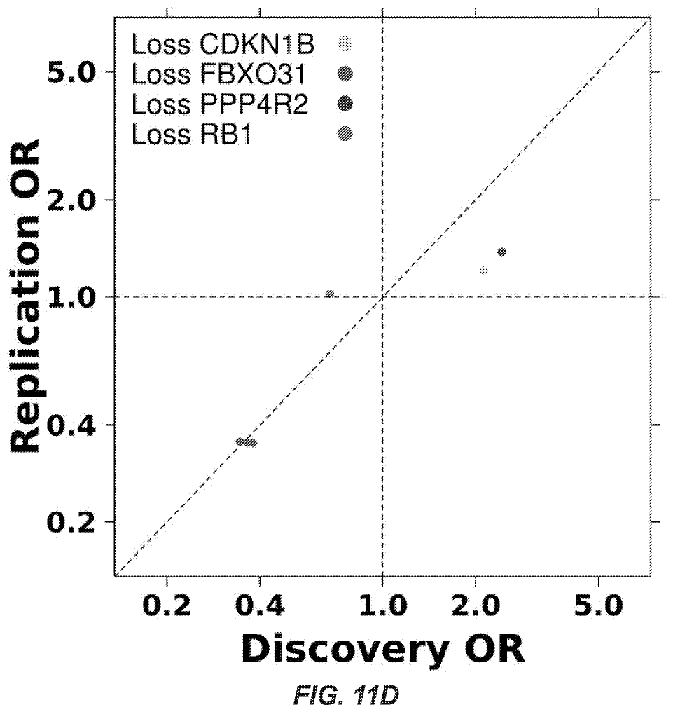

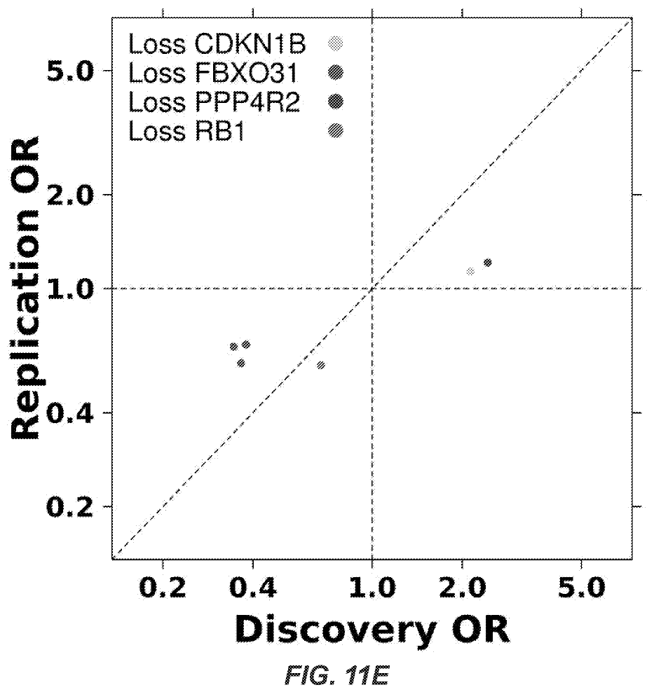

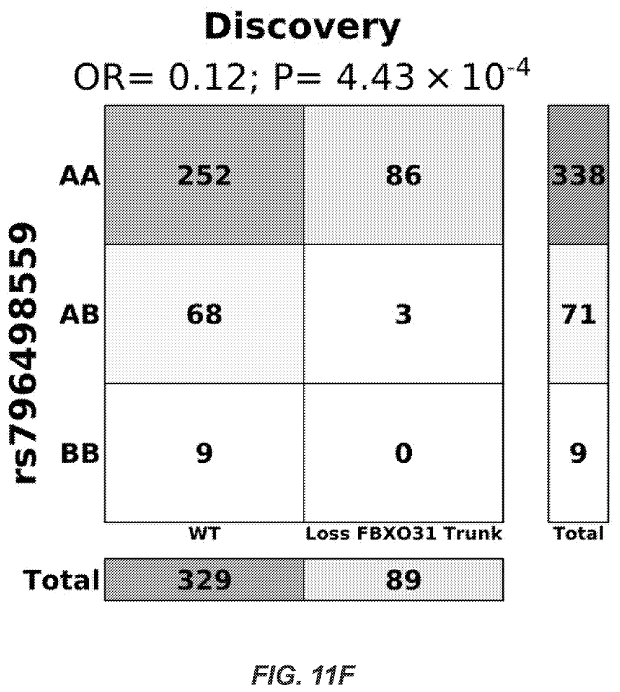

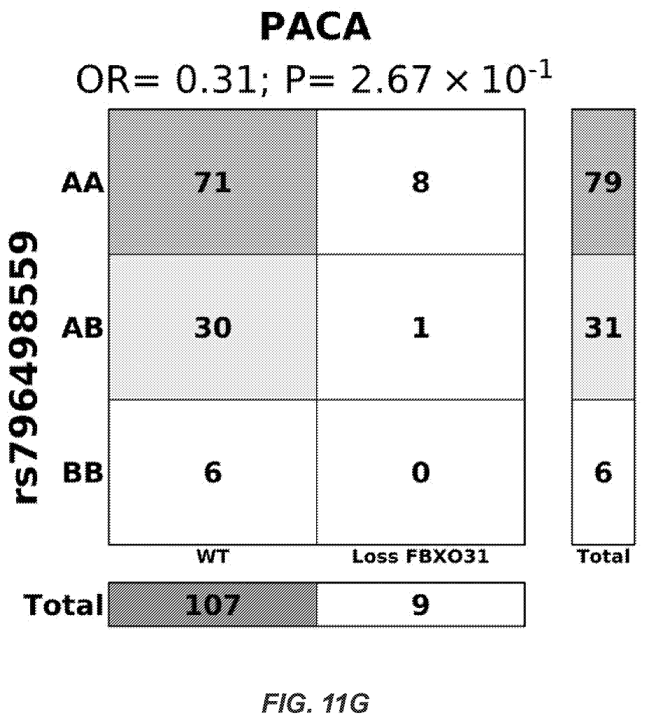

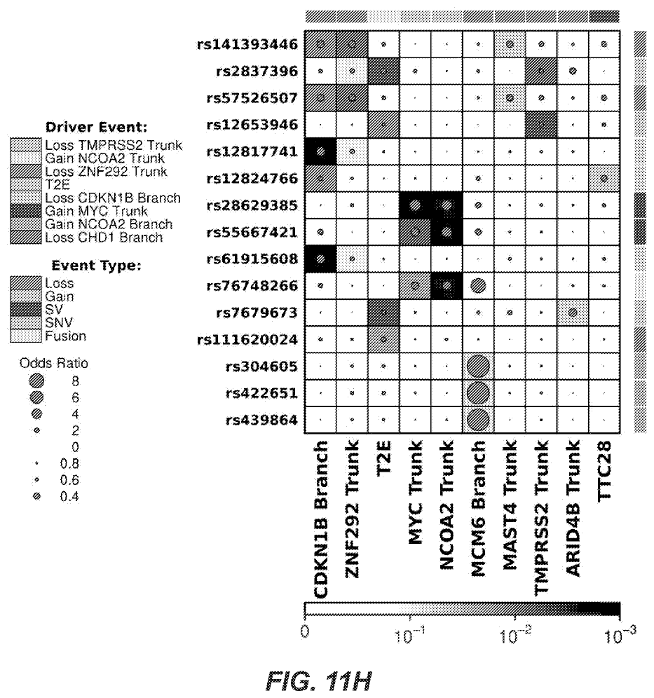

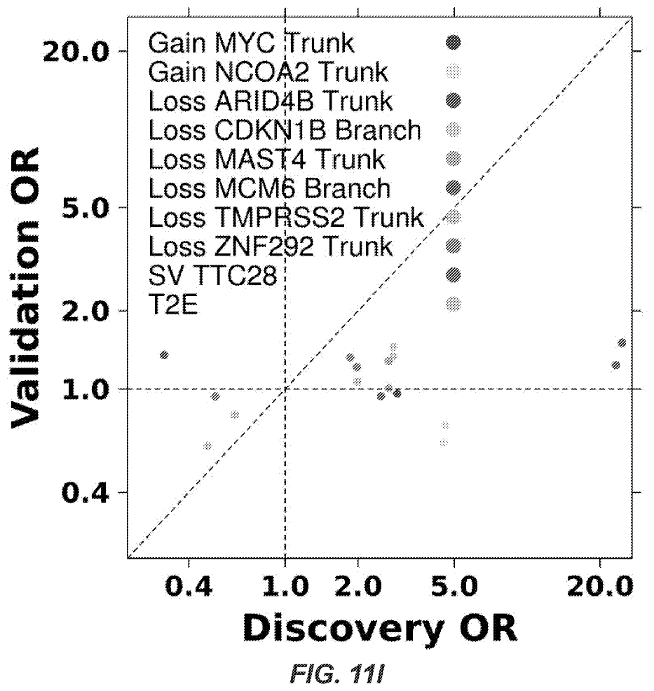

To further explore dQTLs in three-dimensional space, the inventors next considered regions defined by interacting enhancers identified via Hi-ChIP H3K27ac profiling in LNCaP cell lines (FIG. 4A). Briefly, the inventors identified anchor regions outside of the gene boundaries, whose associated anchor fell within the driver gene of interest (see Methods). The 37 somatic drivers were evaluated for associations with 0-1,070 SNPs (median=66; haplotype blocks=0-81; FIG. 11A). The inventors identified 17 dQTLs involving 13 haplotype blocks and eight somatic drivers (logistic regression; Bonferroni α=0.1; P_{unadjusted}<1.27×10⁻²; OR>1.50; FIG. 4B; Table 3). The inventors again successfully verified candidate local dQTLs using array-based data (FIG. 11B).

The inventors defined a tag SNP for each enhancer local dQTL based on the minimum p-value. Of the 13 tag SNPs, eight were profiled in all replication samples, while the other five could only be profiled in the 140 samples with WGS. Nevertheless, 8/13 enhancer local dQTLs showed concordant ORs in both the discovery and replication cohort (FIG. 4C). Two SNPs in strong LD (r²=0.86) associated with FOXA1 3′ UTR somatic SNVs replicated (FIGS. 4D-4G). Unlike for other types of dQTLs, enhancer spatial local dQTLs did not reach statistical significance in ovarian, pancreatic or breast cancer (FIGS. 11C-11E), although most showed consistent ORs (FIGS. 11F-11G). These data suggest that the differing enhancer architecture of different cancer types may make enhancer local dQTLs more likely to be cancer-type specific than linear- or spatial-local dQTLs.

Candidate Distal dQTLs

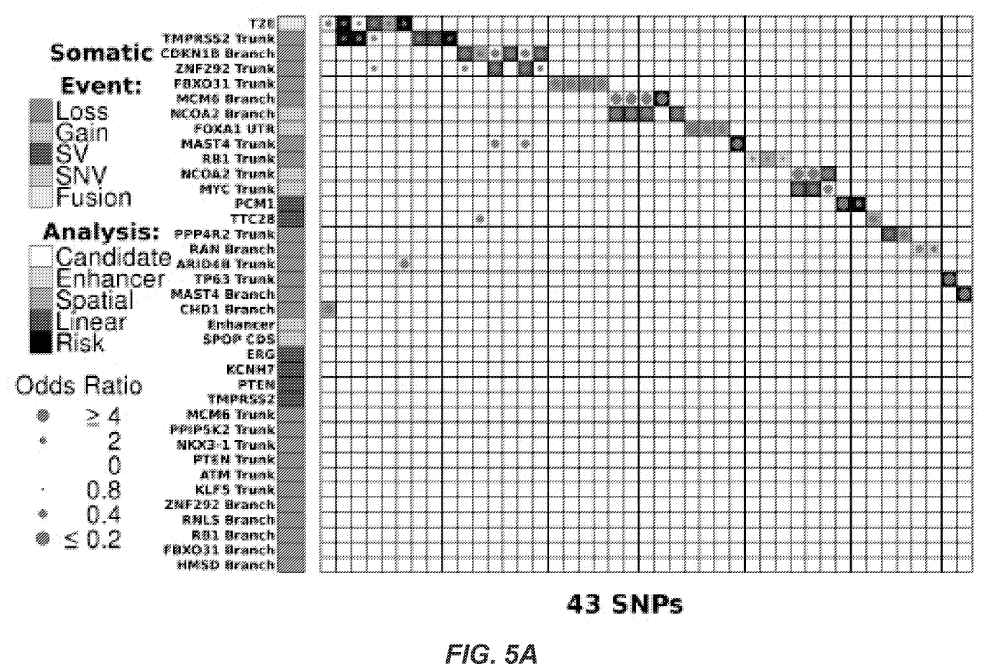

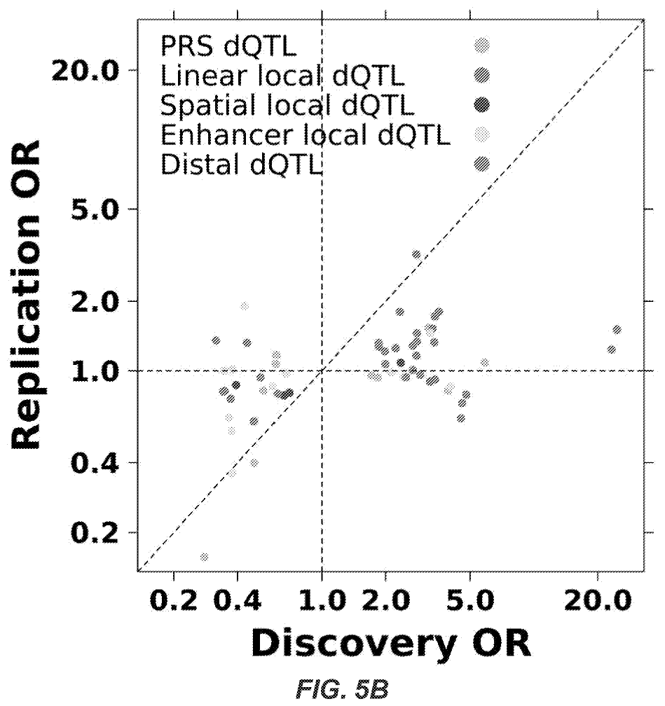

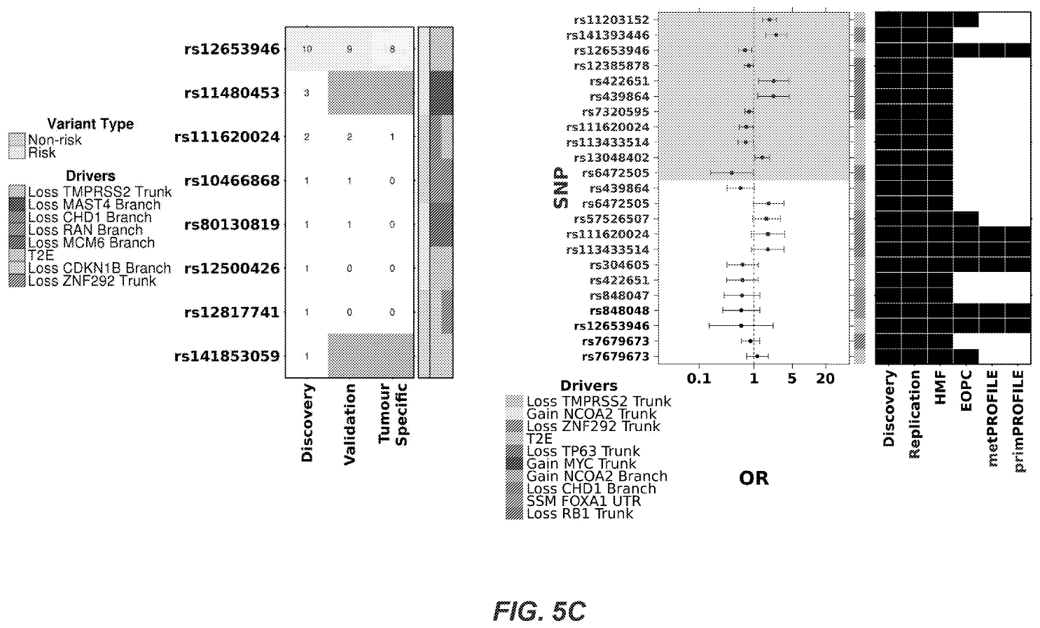

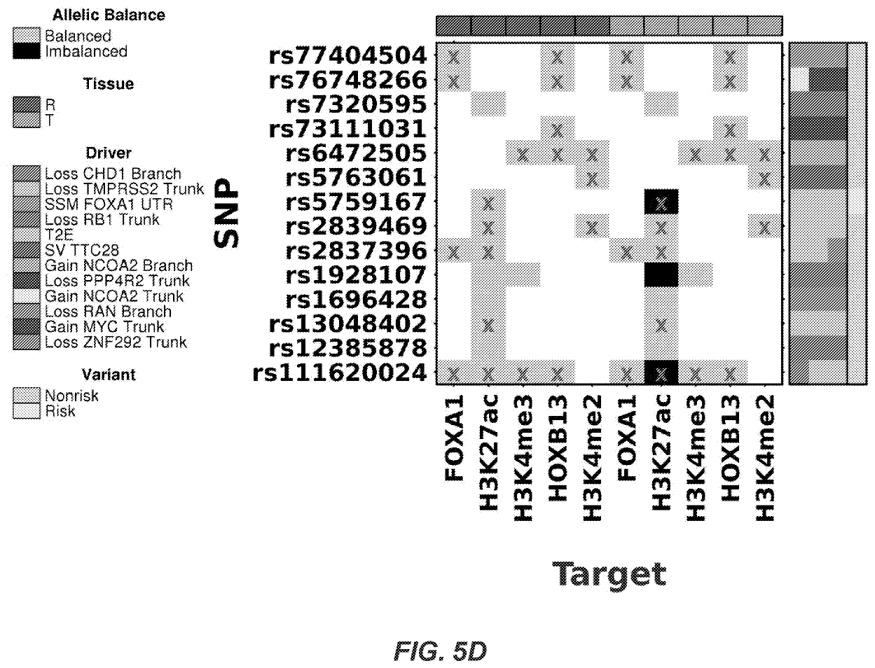

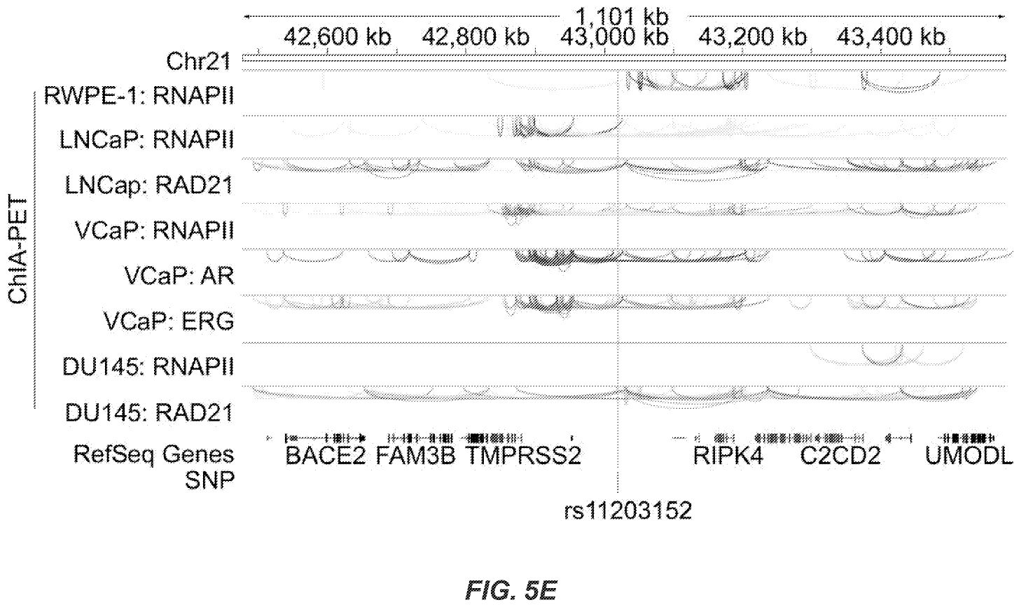

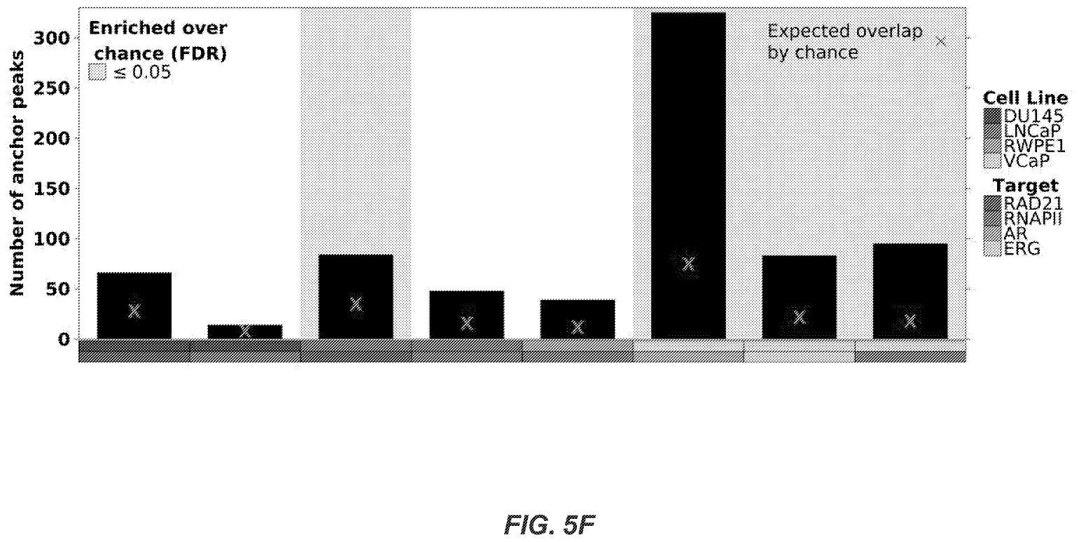

Prostate cancer genomic studies have identified mutually exclusive and co-occurring somatic mutations²⁶. The inventors therefore suspected some local dQTLs might show distal effects for other driver genes. All tag dQTL SNPs from the PRS, linear-local, spatial-local and enhancer-local analyses were merged to yield 43 dQTL tag SNPs. Each of these were screened against the 37 somatic drivers in a candidate analysis. This identified 18 candidate distal dQTLs (FDR<0.1; FIG. 11H; Table 3), 13 of which showed concordant ORs in the inventors' replication cohorts (FIG. 11I). Integrating these results, the inventors discovered 62 dQTLs involving 43 tag SNPs and 19 somatic drivers (FIG. 5A). A majority (69%) showed consistent effect-sizes in the inventors' replication cohort, higher than expected by chance both overall and for tag SNPs (FC=1.32, P=0.038; n=10,000; FIG. 5B). Of these 62 dQTLs, seven were directly replicated in one or more other cancer types in the PCAWG cohort.

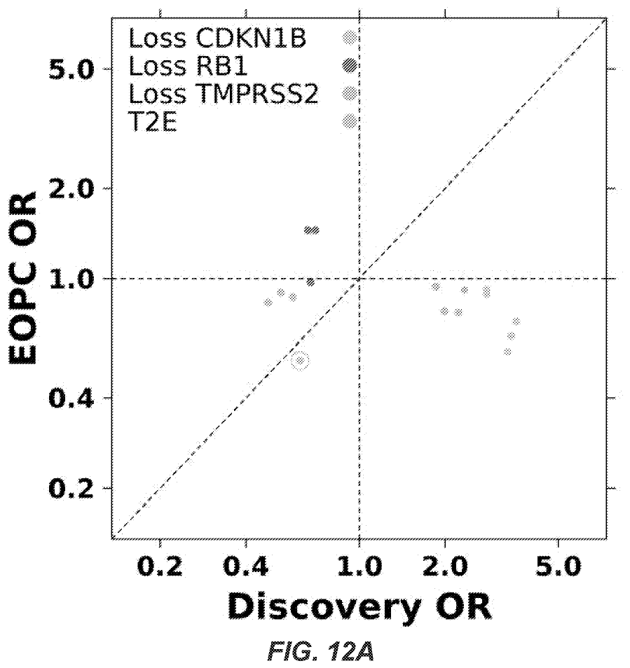

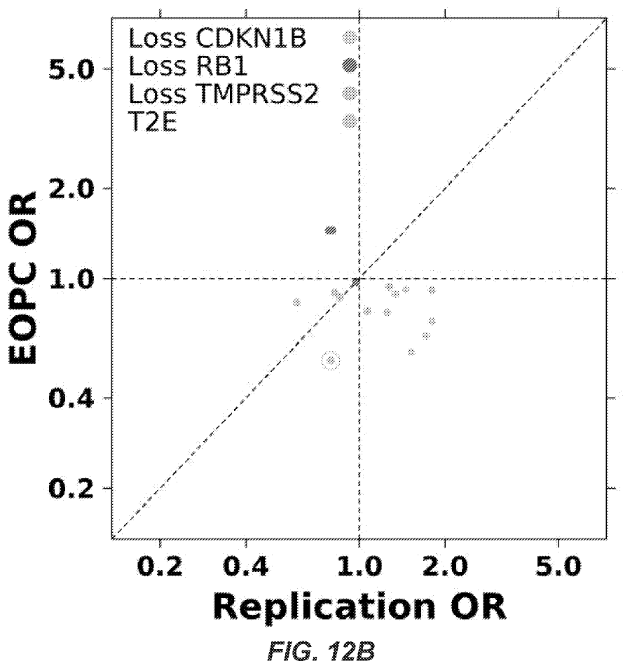

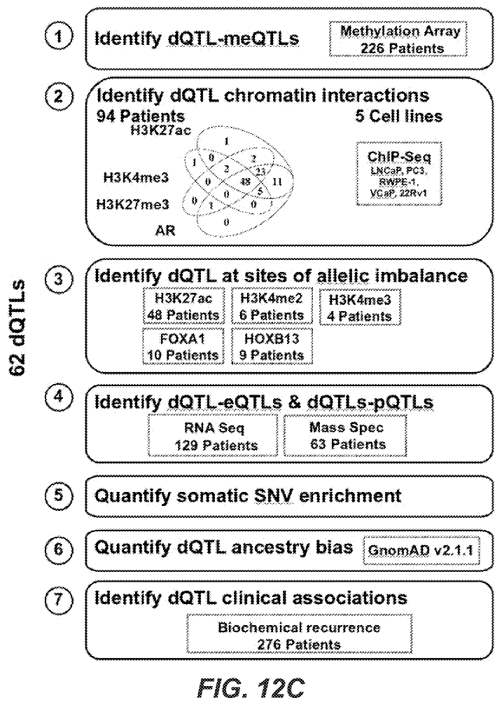

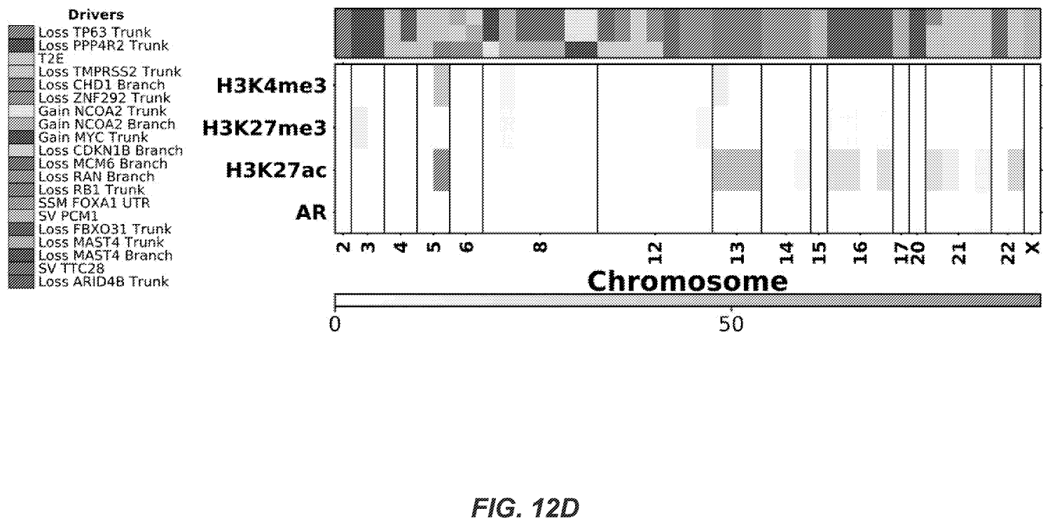

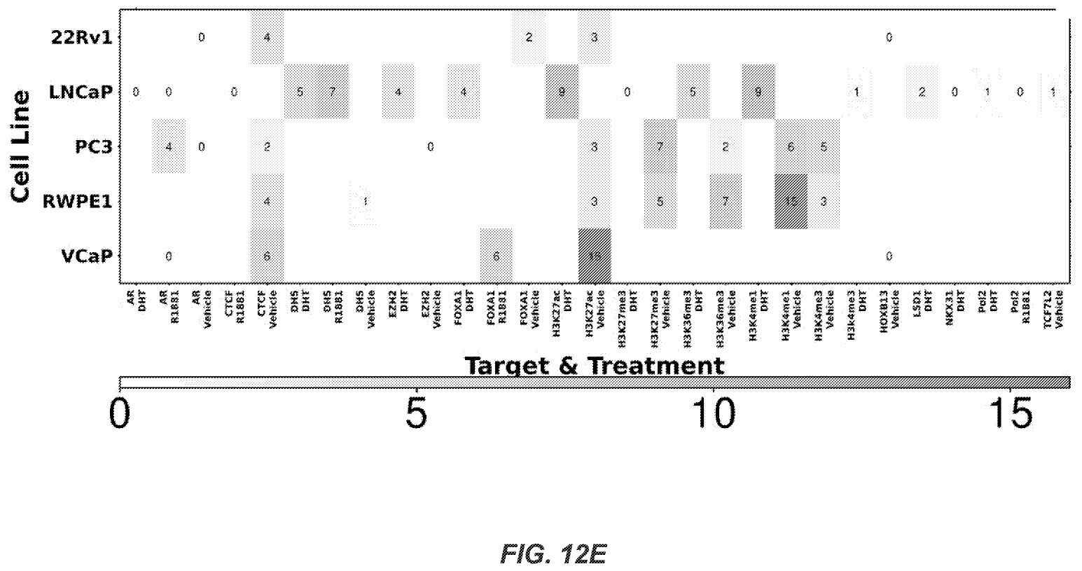

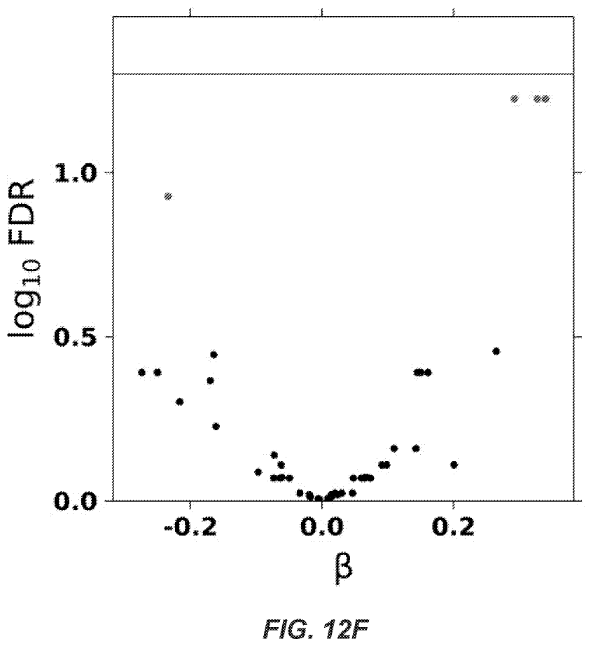

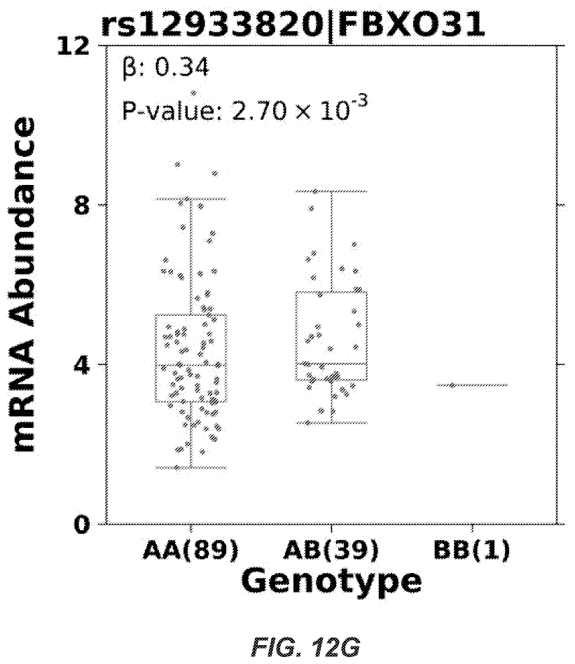

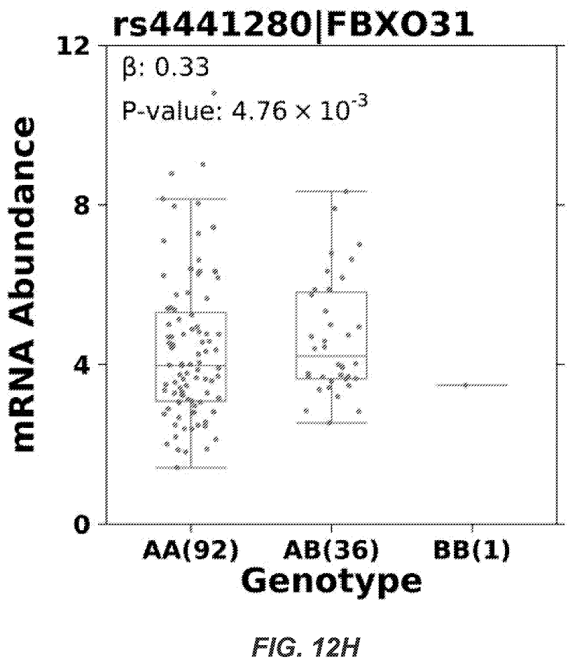

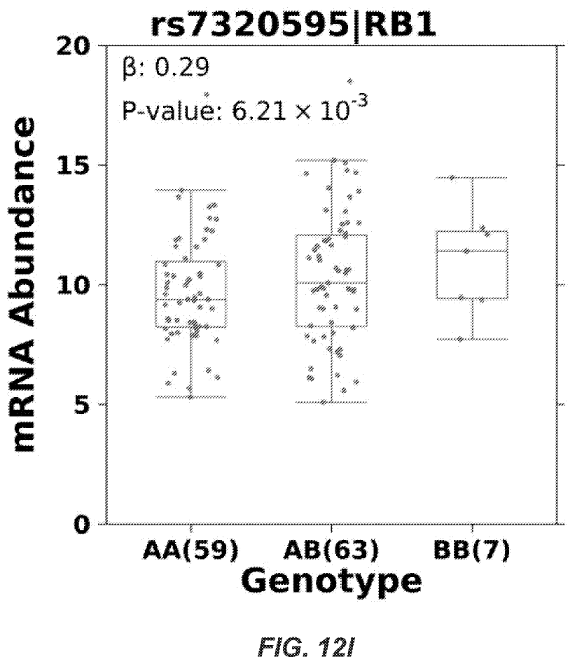

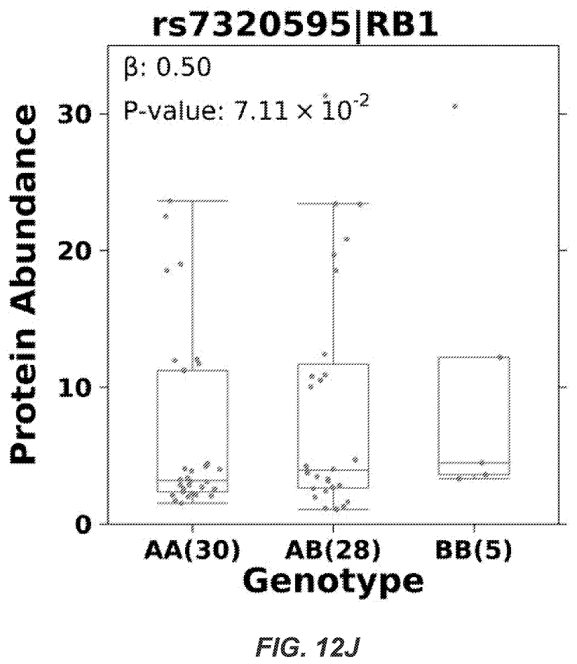

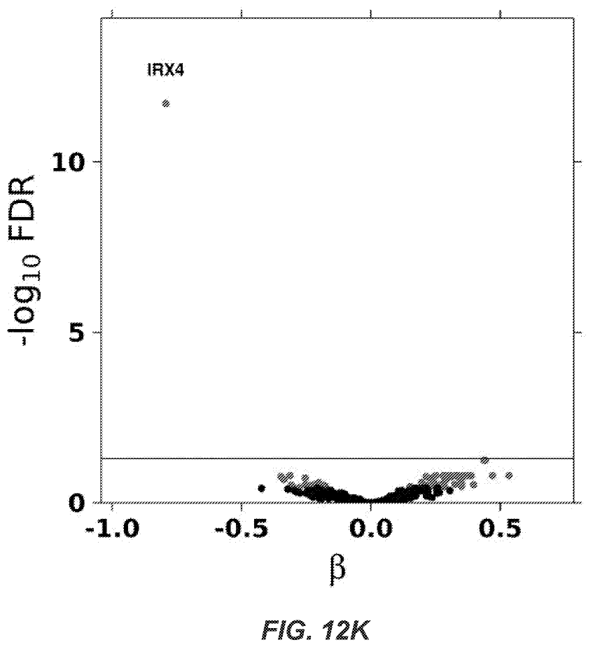

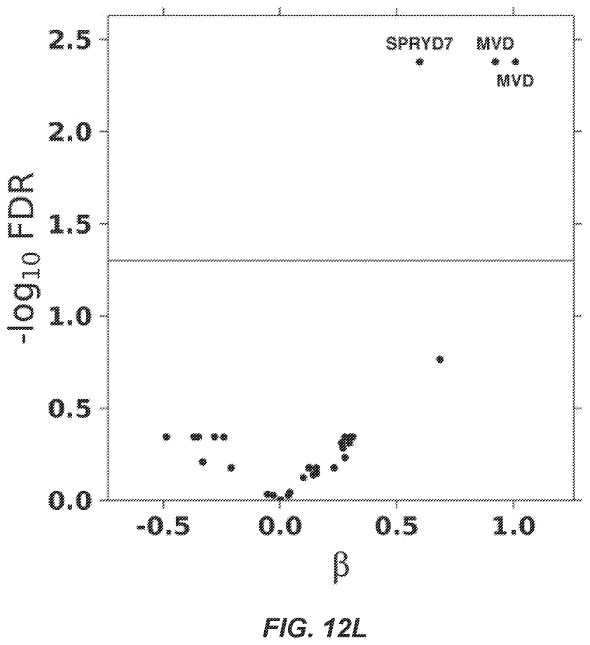

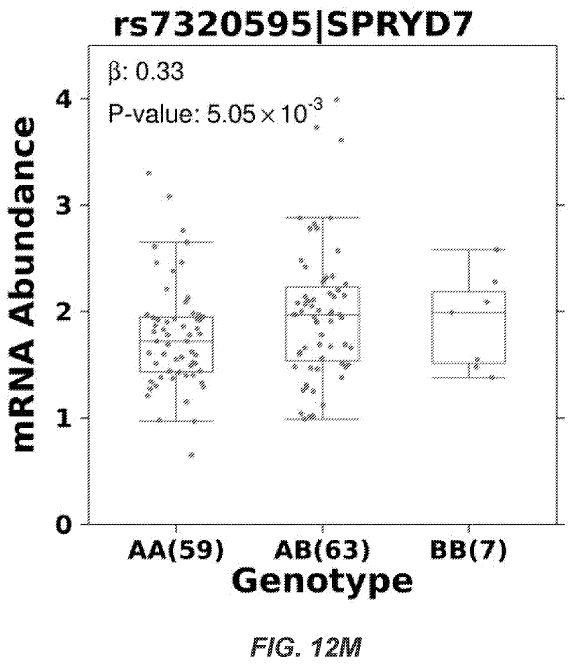

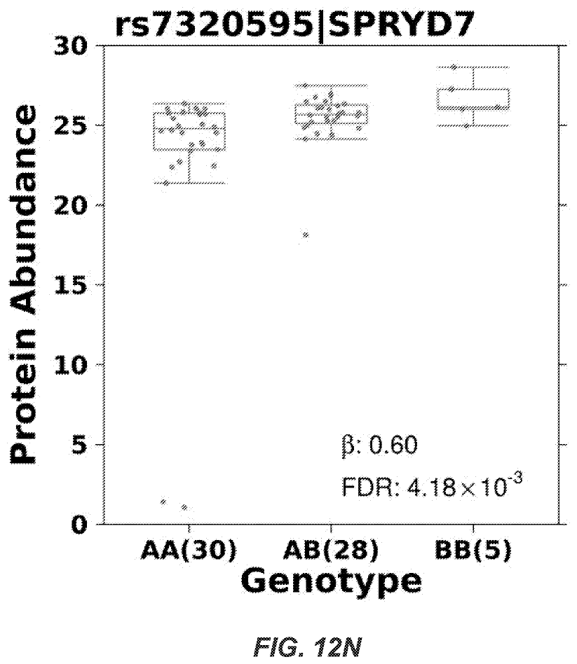

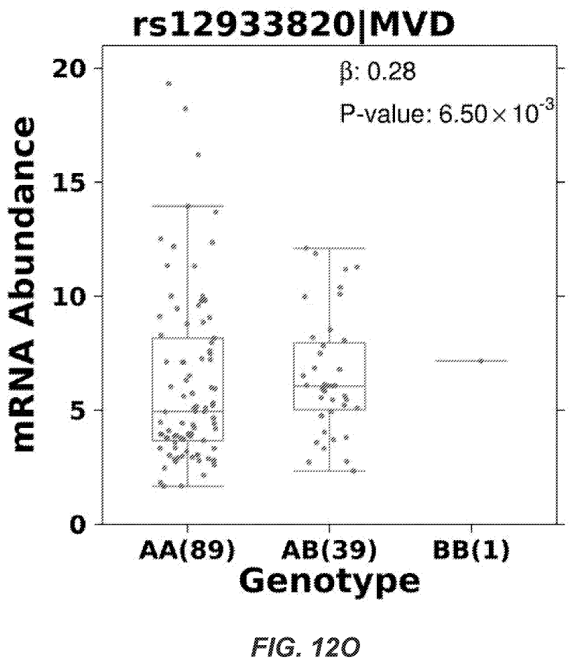

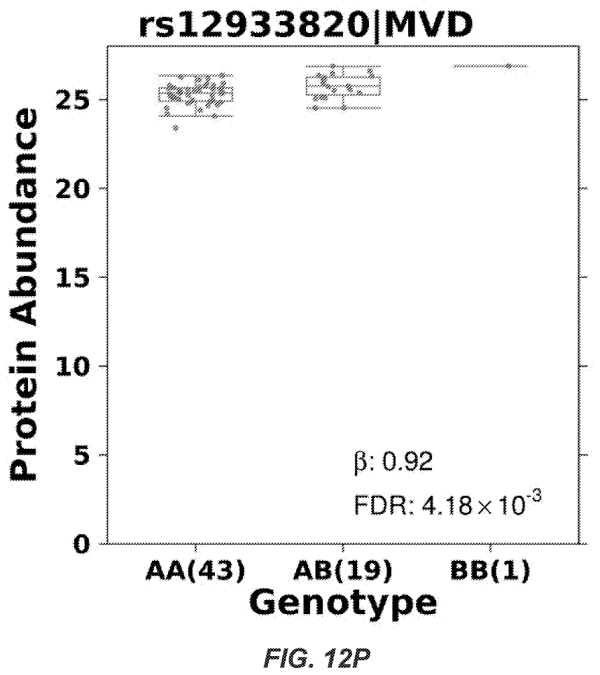

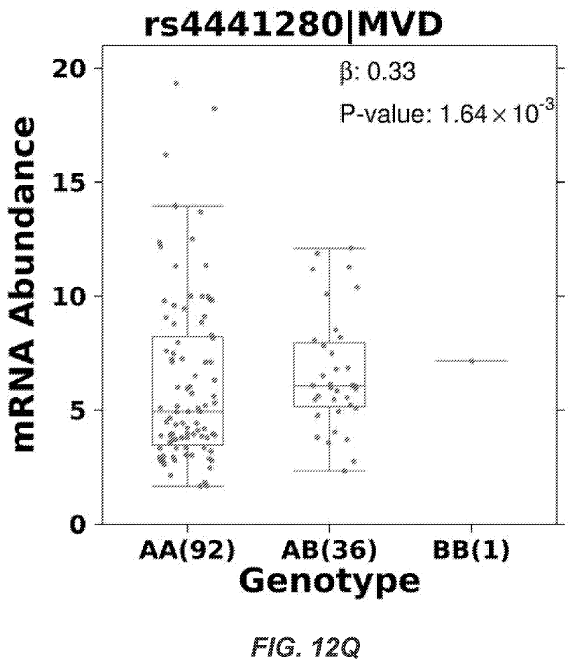

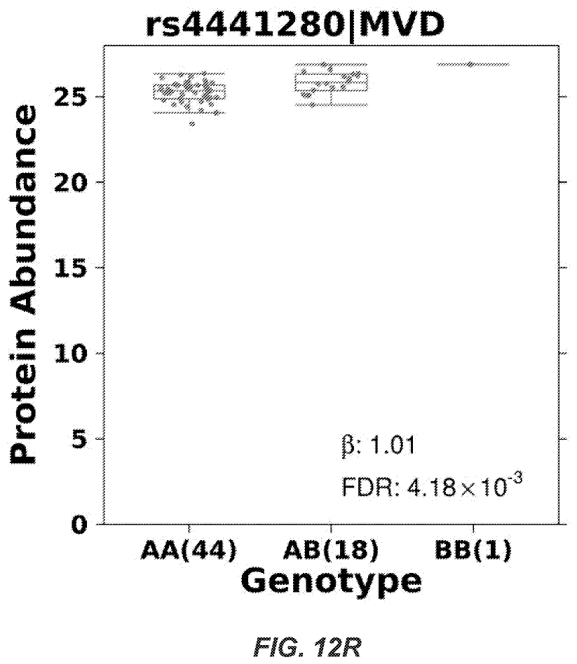

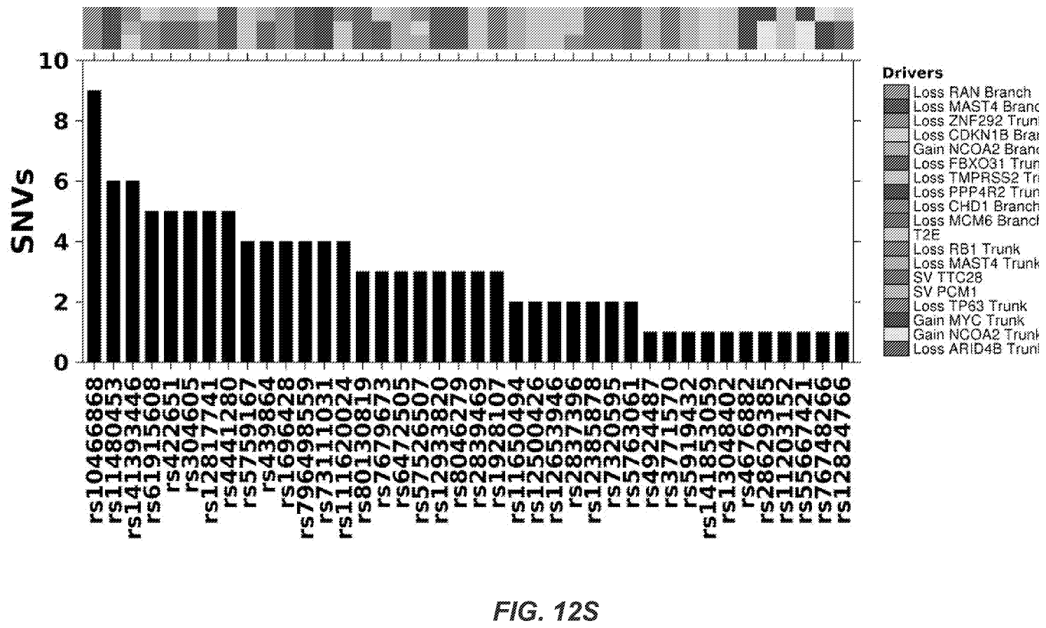

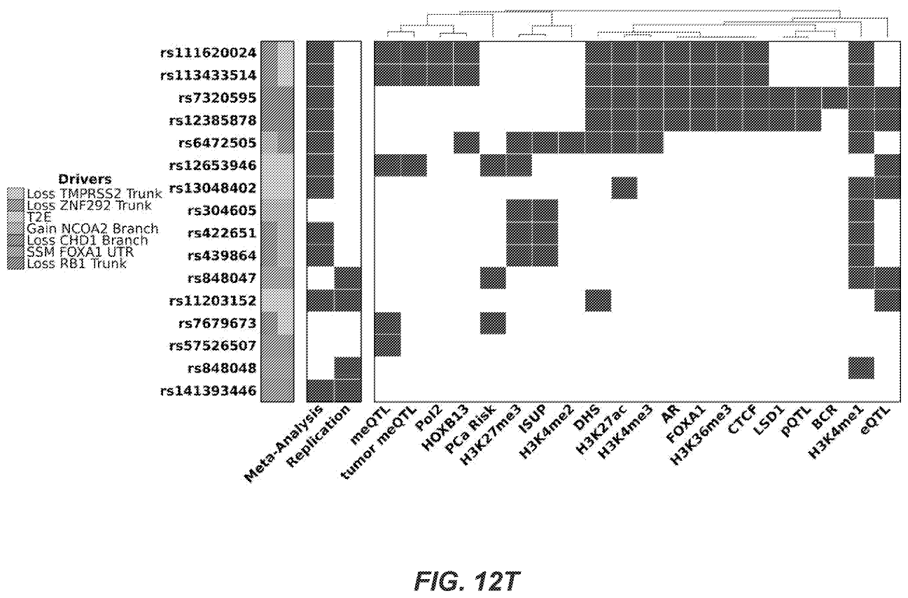

Finally, to extend these results to other forms of prostate cancer, the inventors considered early-onset prostate tumors (EOPC; diagnosis <55 years). The inventors evaluated the 16 dQTLs associated with somatic drivers occurring in at least 5% of this disease in 238 patients with tumour and reference WGS³⁸. Only 5/16 showed consistent effect directions in EOPC as in late-onset prostate cancer. The previously identified genetic³⁴ association between rs12653946 and T2E was replicated in EOPC (OR_{discovery}=0.62; FDR_{discovery}=0.032; OR_{EOPC}=0.53; P_{EOPC}=3.57×10⁻³; FIGS. 12A-12B). These data suggest dQTLs may be as divergent between influencing different diseases at a single histologic site as between cancer types, although additional replication studies and larger cohorts will be needed to understand this relationship systematically.

Local dQTLs Modulate the Tumor Epigenome

Deregulation of tumor methylation is one mechanism by which the germline genome influences cancer risk^{19,20}, so the inventors investigated if any of the 43 dQTL tag SNPs were associated with methylation changes in tumor tissue (FIG. 12C). The inventors conducted a candidate local meQTL analysis and evaluated associations with methylation±10 kbp around the dQTL tag SNPs. The inventors leveraged array-based methylation profiling for 226 patients from the discovery cohort and 412 patients from the replication cohort, along with 47 additional matched profiles of histologically non-malignant reference prostate tissue. This candidate analysis identified 20 local meQTLs involving eight dQTLs (|β_{discovery}|>0.087; FDR_{discovery}<0.05). Of these, 18 local meQTLs were also genotyped in the inventors' replication cohort, and 13 replicated (|β_{replication}|>0.14; FDR_{replication}<4.65×10⁻³; FIG. 5C; Table 4). Two SNPs, rs12653946 associated with T2E and clonal loss of TMPRSS2 and rs111620024 associated with T2E and subclonal loss of CHD1, were involved in tumor-specific meQTLs, meaning these SNPs were associated with methylation changes in tumor tissue but not reference tissue²⁰ (|βₜᵤₘₒᵣ|>0.21; FDRₜᵤₘₒᵣ<3.42×10⁻²; |β_{reference}|<0.62; FDR_{reference}>0.074).

To explore if dQTLs are associated with broader changes in the tumor epigenome, the inventors evaluated their association with histone marks in primary prostate tumors for H3K27ac (n=92 patients), H3K27me3 (n=76) and H3K4me3 (n=56) and androgen receptor (AR; n=88) binding³⁹ (FIG. 12C). A subset of dQTLs target active regulatory regions: 15 dQTLs overlap H3K27ac modification sites (2-89 patients) and six overlap H3K4me3 (1-47 patients) of which four also overlap H3K27ac sites (FIG. 12D; Table 4). Five enhancer dQTLs overlap H3K27ac modification sites in primary prostate tumours supporting the inventors' use of cell-line H3K27ac HiChIP profiling to identify interacting enhancers. Out of the 13 dQTLs that overlap H3K27me3, three overlapped H3K27ac sites in other patients indicative of bivalent chromatin regions. Finally, dQTLs also overlap master transcriptional regulators of prostate cancer—eight dQTLs overlapped AR binding sites (2-55 patients). The inventors replicated these findings in a second cohort of 48 primary prostate cancer tumors profiled via ChIP-Seq for H3K27ac (n=48 patients), H3K4me2 (n=6 patients), H3K4me3 (n=4 patients), FOXA1 (n=10 patients) and HOXB13 (n=9 patients; FIG. 5D; FIG. 12C; Table 4). Further, of the 43 dQTL tag SNPs, 35 overlapped with active regulatory regions and master transcription factor binding sites in four cancer cell lines and one prostate epithelial cell line (FIGS. 12C and 12E; Table 4)⁴⁰⁻⁵³. While the overlap of dQTLs at transcription-factor bindings sites or regulatory chromatin domains was not more than expected by chance (P>0.57), these results suggest a subset of dQTLs may modulate local determinants of somatic mutations⁵⁴. These data are consistent with increased prostate cancer risk heritability and somatic mutation density in these regions⁵⁵.

To quantify regulatory potential of dQTLs, the inventors identified sites of allelic imbalance in ChIP-Seq peaks for the 48 primary prostate cancer tumors introduced above. Three out of nine dQTLs at H3K27ac modification sites demonstrated allelic imbalance (FIG. 5D; Table 4). This imbalance was specifically detected in tumor tissue and absent in normal tissue, supporting tumor-specific dQTL regulatory roles.

Finally, to begin to elucidate a mechanism of dQTLs the inventors focused on the impact of rs11203152—associated with loss of TMPRSS2—on local chromatin structure. This SNP is in close proximity to multiple chromatin looping sites anchored by RNA Polymerase II (RNAPII), RAD21, AR and ERG in prostate cancer cell lines (FIG. 5E). There was a clear enrichment of RAD21 chromatin loop anchors around rs11203152 in LNCaP cells (FDR=0.04; observed number of anchors=84; expected=35) but not DU145 cells (FDR=0.19; observed=66; expected=28; FIG. 5F). Further, VCaP cells, which have a T2E fusion, showed an enrichment of RNA Polymerase II (FDR=0.04; observed=95; expected=18), AR (FDR=0.04; observed=325; expected=75) and ERG (FDR=0.04; observed=83; expected=22) anchored chromatin loops around rs11203152 (FIG. 5F). Altogether, these data suggest rs11203152 may interact with AR regulation to influence loss of TMPRSS2.

TABLE 4
Summary of characterization of all 62 dQTLs.

| SNP | Gene | Phenotype (Genetic abnormality) | chromosome |
|---|---|---|---|
| rs28629385 | MYC | Increased copy number | 8 |
| rs55667421 | MYC | Increased copy number | 8 |
| rs76748266 | MYC | Increased copy number | 8 |
| rs304605 | NCOA2 | Increased copy number | 8 |
| rs422651 | NCOA2 | Increased copy number | 8 |
| rs439864 | NCOA2 | Increased copy number | 8 |
| rs6472505 | NCOA2 | Increased copy number | 8 |
| rs28629385 | NCOA2 | Increased copy number | 8 |
| rs55667421 | NCOA2 | Increased copy number | 8 |
| rs76748266 | NCOA2 | Increased copy number | 8 |
| rs7679673 | ARID4B | Reduced expression and/or deletion | 4 |
| rs12817741 | CDKN1B | Reduced expression and/or deletion | 12 |
| rs12824766 | CDKN1B | Reduced expression and/or deletion | 12 |
| rs141393446 | CDKN1B | Reduced expression and/or deletion | 6 |
| rs141853059 | CDKN1B | Reduced expression and/or deletion | 12 |
| rs57526507 | CDKN1B | Reduced expression and/or deletion | 6 |
| rs61915608 | CDKN1B | Reduced expression and/or deletion | 12 |
| rs111620024 | CHD1 | Reduced expression and/or deletion | 5 |
| rs12933820 | FBXO31 | Reduced expression and/or deletion | 16 |
| rs4441280 | FBXO31 | Reduced expression and/or deletion | 16 |
| rs796498559 | FBXO31 | Reduced expression and/or deletion | 16 |
| rs8046279 | FBXO31 | Reduced expression and/or deletion | 16 |
| rs11480453 | MAST4 | Reduced expression and/or deletion | 20 |
| rs11650494 | MAST4 | Reduced expression and/or deletion | 17 |
| rs141393446 | MAST4 | Reduced expression and/or deletion | 6 |
| rs57526507 | MAST4 | Reduced expression and/or deletion | 6 |
| rs304605 | MCM6 | Reduced expression and/or deletion | 8 |
| rs422651 | MCM6 | Reduced expression and/or deletion | 8 |
| rs439864 | MCM6 | Reduced expression and/or deletion | 8 |
| rs80130819 | MCM6 | Reduced expression and/or deletion | 12 |
| rs4676882 | PPP4R2 | Reduced expression and/or deletion | 3 |
| rs73111031 | PPP4R2 | Reduced expression and/or deletion | 3 |
| 12:131939920G, T | RAN | Reduced expression and/or deletion | 12 |
| rs1696428 | RAN | Reduced expression and/or deletion | 12 |
| rs12385878 | RB1 | Reduced expression and/or deletion | 13 |
| rs1928107 | RB1 | Reduced expression and/or deletion | 13 |
| rs7320595 | RB1 | Reduced expression and/or deletion | 13 |
| rs11203152 | TMPRSS2 | Reduced expression and/or deletion | 21 |
| rs12500426 | TMPRSS2 | Reduced expression and/or deletion | 4 |
| rs12653946 | TMPRSS2 | Reduced expression and/or deletion | 5 |
| rs13048402 | TMPRSS2 | Reduced expression and/or deletion | 21 |
| rs2837396 | TMPRSS2 | Reduced expression and/or deletion | 21 |
| rs5759167 | TMPRSS2 | Reduced expression and/or deletion | 22 |
| rs3771570 | TP63 | Reduced expression and/or deletion | 2 |
| rs12817741 | ZNF292 | Reduced expression and/or deletion | 12 |
| rs141393446 | ZNF292 | Reduced expression and/or deletion | 6 |
| rs2837396 | ZNF292 | Reduced expression and/or deletion | 21 |
| rs57526507 | ZNF292 | Reduced expression and/or deletion | 6 |
| rs61915608 | ZNF292 | Reduced expression and/or deletion | 12 |
| rs77404504 | FOXA1 | Simple somatic mutation (single nucleotide | 14 |
| | | variant or indel) in UTR | |
| rs848047 | FOXA1 | Simple somatic mutation (single nucleotide | 14 |
| | | variant or indel) in UTR | |
| rs848048 | FOXA1 | Simple somatic mutation (single nucleotide | 14 |
| | | variant or indel) in UTR | |
| rs4924487 | PCM1 | Structural variant | 15 |
| rs5919432 | PCM1 | Structural variant | X |
| rs5919432 | PCM1 | Structural variant | X |
| rs12824766 | TTC28 | Structural variant | 12 |
| rs5763061 | TTC28 | Structural variant | 22 |
| rs111620024 | TMPRSS2 | T2E (TMPRSS2-ERG fusion) | 5 |
| rs12500426 | TMPRSS2 | T2E (TMPRSS2-ERG fusion) | 4 |
| rs12653946 | TMPRSS2 | T2E (TMPRSS2-ERG fusion) | 5 |
| rs2837396 | TMPRSS2 | T2E (TMPRSS2-ERG fusion) | 21 |
| rs2839469 | TMPRSS2 | T2E (TMPRSS2-ERG fusion) | 21 |
| rs7679673 | TMPRSS2 | T2E (TMPRSS2-ERG fusion) | 4 |

dQTLs Modulate Tumor Gene Expression

Given the overlap of dQTLs in areas of active chromatin, the inventors sought to quantify their influence on tumor gene expression. First, the inventors assessed if any dQTL tag SNPs were expression quantitative trait loci (eQTL) for their associated somatic driver gene (FIG. 12F). The inventors identified two dQTL-eQTLs associated with RB1 mRNA abundance and three with FBXO31 mRNA abundance (FDR<0.1; FIGS. 12G-12I; Table 4). FBXO31 protein abundance was not quantified in the inventors' cohort, however, both rs12385878 and rs7320595 were associated with RB1 protein abundance (β=0.29; P=7.71×10⁻²; FIG. 12J). To expand eQTL discovery beyond somatic driver genes the inventors evaluated genes in close proximity to the dQTL, defined as ±1.0 Mbp. To the inventors' surprise, only a single eQTL was significant after correcting for multiple hypothesis testing: rs12653946−IRX4⁵⁶ (β=−0.80; FDR=3.17×10⁻¹³; FIG. 12K; Table 4). By contrast, of the 76 nominally significant eQTLs (P<0.05), five were identified as genome-wide significant protein QTLs (pQTLs; FDR<0.05; FIG. 12L). Two SNPs in strong LD (r²=0.97), rs7320595 and rs12385878, were associated with SPRYD7 mRNA (β=0.33; P=5.5×10⁻³; FIG. 12M) and protein abundance (P=0.60; FDR=4.18×10⁻³; FIG. 12N). Three SNPs were associated with MVD mRNA (FIGS. 12O and 12Q) and protein abundance (βᵣₛ₁₂₉₃₃₈₂₀=0.92; FDRᵣₛ₁₂₉₃₃₈₂₀=4.18×10⁻³; βᵣₛ₄₄₄₁₂₈₀=1.01; FDRᵣₛ₄₄₄₁₂₈₀=4.18×10⁻³; FIGS. 12P and 12R). To determine if there was broader transcriptome modeling, the inventors evaluated dQTL tag SNPs for association with mRNA abundance genome-wide. Only rs12653946—IRX4⁵⁶ was genome-wide significant. Finally, the inventors leveraged Genotype-Tissue Expression (GTEx)⁵⁷ project to evaluate if dQTL tag SNPs were associated with mRNA abundance in non-malignant prostate tissue. Five dQTLs were involved in normal tissue eQTLs, including rs12653946—IRX4 (P<3.8×10⁻⁵; Table 5). Thus a subset of dQTLs modulate the tumor transcriptome and proteome.

TABLE 5
dQTL SNPs identified as eQTLs in prostate tissue in GTEx.

| Gencode. Id | Gene. Symbol | Variant. Id | SNP. Id | P. Value | NES | Tissue |
|---|---|---|---|---|---|---|
| ENSG00000102543.14 | CDADC1 | chr13: 49282062 C > A | rs12385878 | 0.0000049 | −0.25 | Prostate |
| ENSG00000275202.1 | RP11-156K23.3 | chr13: 49282062 C > A | rs12385878 | 0.000007 | −0.4 | Prostate |
| ENSG00000138696.10 | BMPR1B | chr4: 94593458A > C | rs12500426 | 0.0000051 | 0.15 | Prostate |
| ENSG00000113430.9 | IRX4 | chr5: 1895715 C > T | rs12653946 | 1E−14 | −0.4 | Prostate |
| ENSG00000249116.1 | CTD-2194D22.3 | chr5: 1895715 C > T | rs12653946 | 1.1E−09 | −0.35 | Prostate |
| ENSG00000275202.1 | RP11-156K23.3 | chr13: 49001371 T > C | rs1928107 | 4.3E−22 | −0.76 | Prostate |
| ENSG00000275202.1 | RP11-156K23.3 | chr13: 49281140 C > T | rs7320595 | 0.0000032 | −0.42 | Prostate |
| ENSG00000102543.14 | CDADC1 | chr13: 49281140 C > T | rs7320595 | 0.000038 | −0.23 | Prostate |

dQTLs Preferentially Occur in Regions Enriched for Somatic SNVs

Next, the inventors reasoned that if dQTLs provide a fitness advantage, tumours might acquire a similar advantage via somatic mutations as well⁵⁸ (FIG. 12C). To test this hypothesis, the inventors evaluated whether somatic mutations were enriched within the region of individual dQTLs. The inventors focused on the 40 dQTL tag SNPs associated with CNAs and GRs, and identified somatic SNVs within ±10 kbp of each. The median dQTLs had three proximal somatic SNVs in this window across the cohort, more than the ˜0.5 expected by chance alone (range: 0-9; P=3.0×10⁻⁴; permutation analysis n=10,000; FIG. 12S). A similar enrichment of somatic SNVs within ±10 kbp of these dQTLs was observed in breast (median_{dQTLs}=9; medianₙᵤₗₗ=4; P=3.0×10⁻³), pancreatic (median_{dQTLs}=9; medianₙᵤₗₗ=2.5; P=0) and ovarian cancer (median_{dQTLs}=6; medianₙᵤₗₗ=2; P=1.0×10⁻³). This suggests that dQTLs may confer fitness advantage that tumors can also acquire via somatic SNVs.

dQTL Allelic Frequencies are Biased Across Ancestry Populations

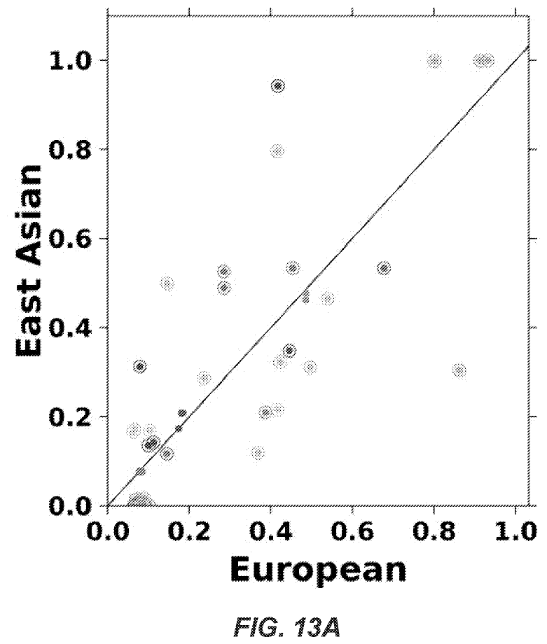

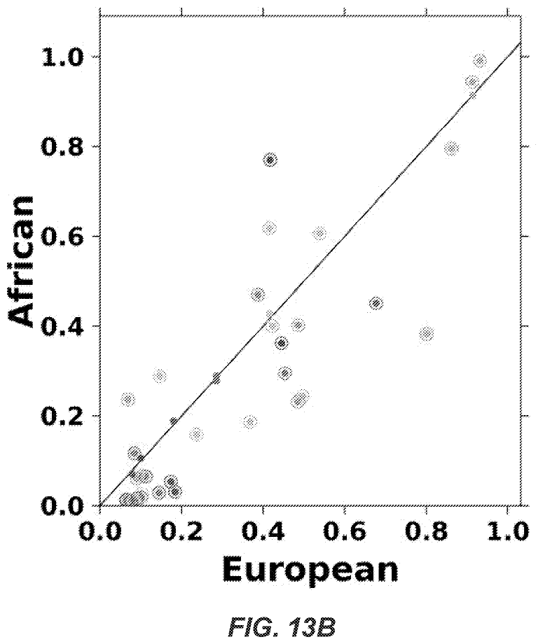

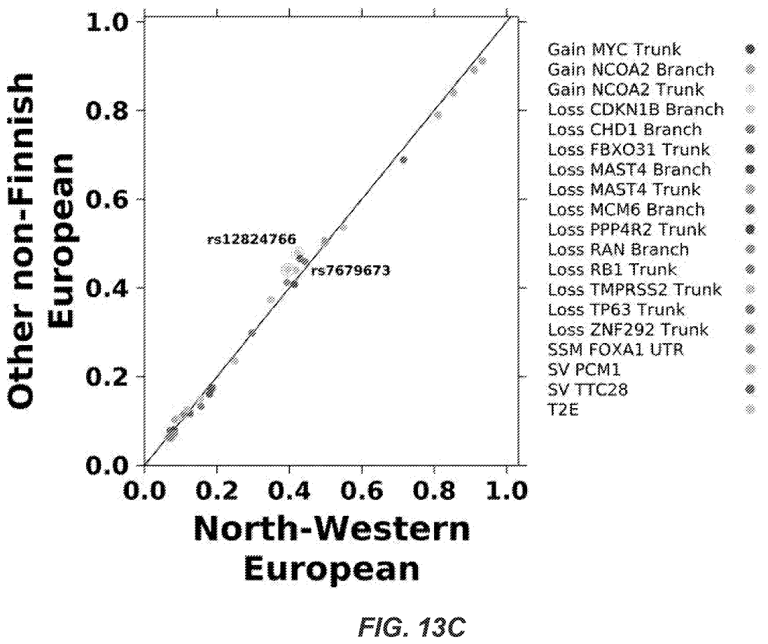

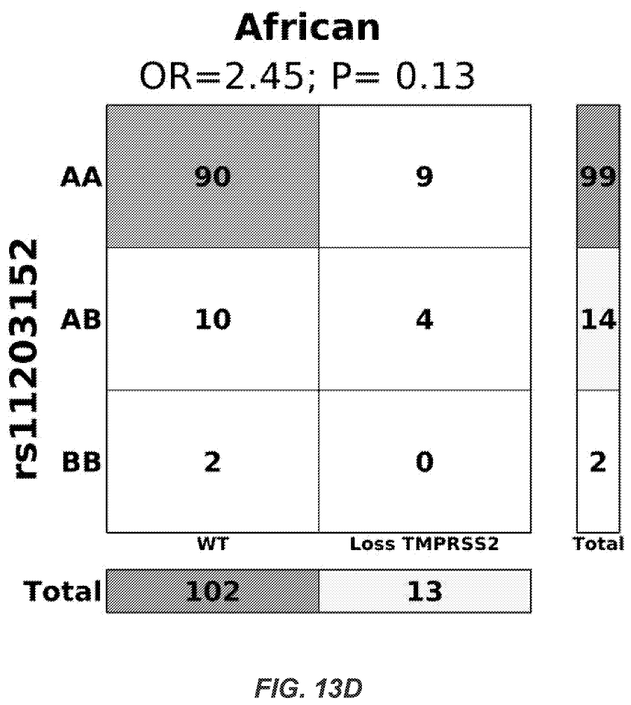

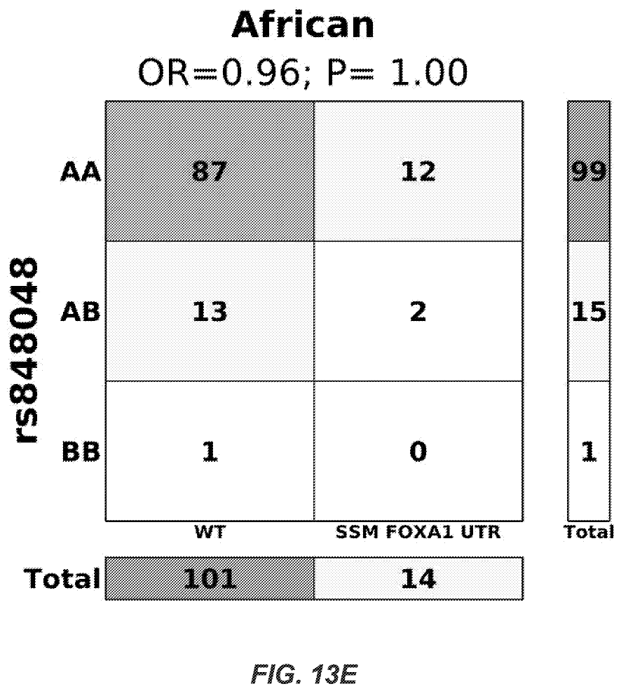

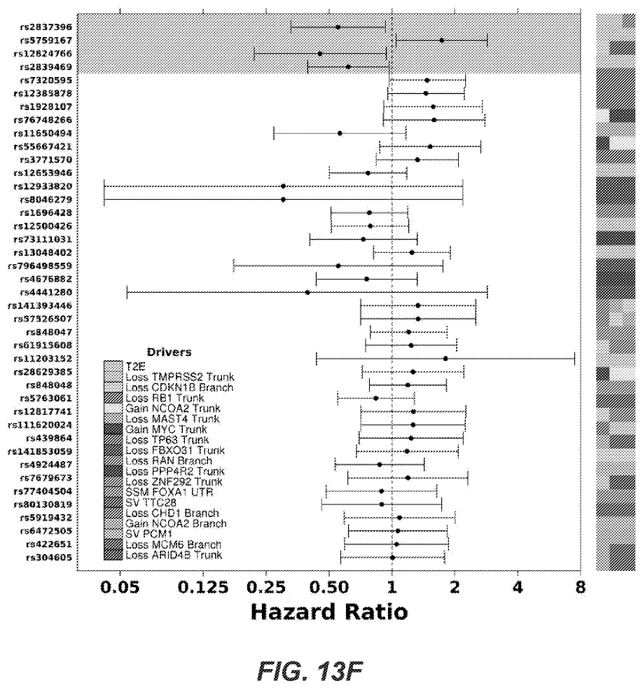

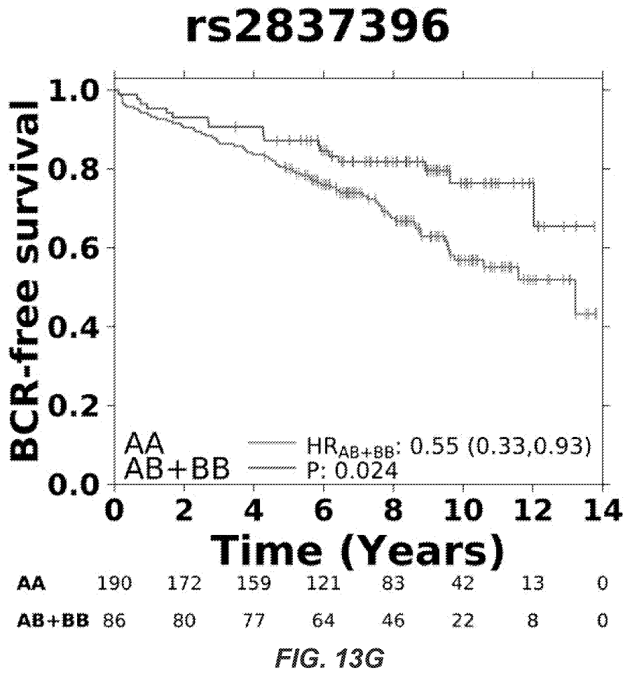

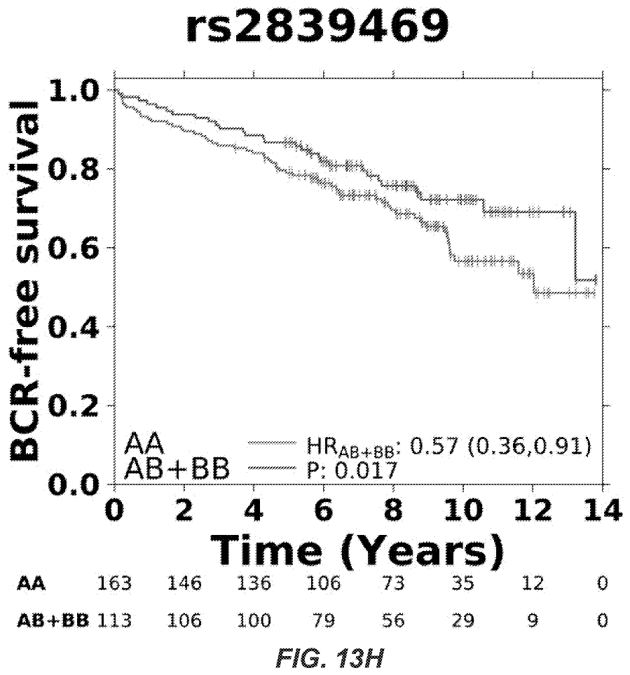

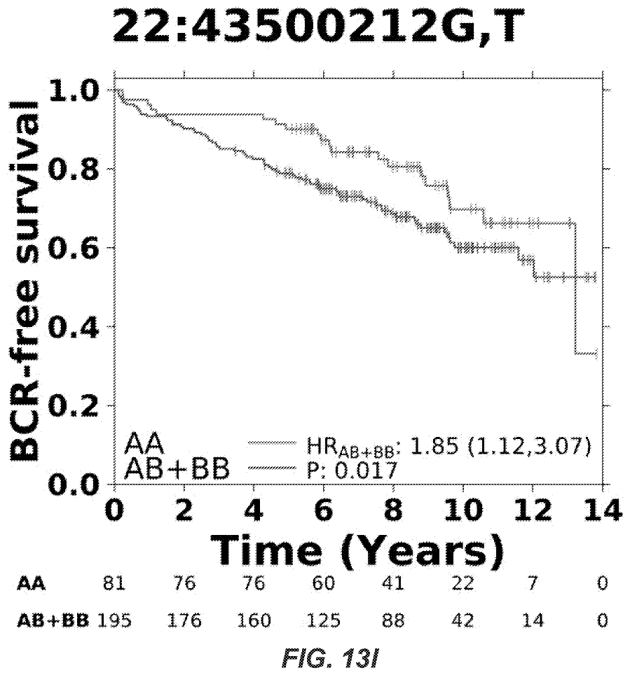

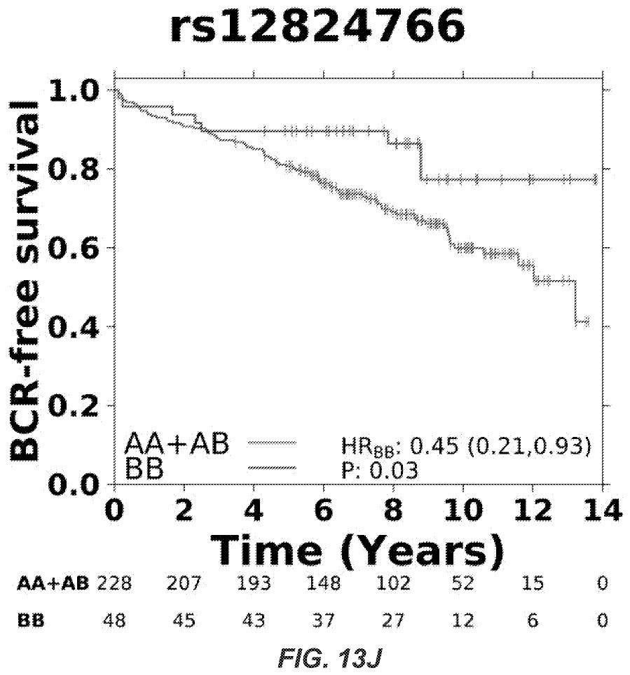

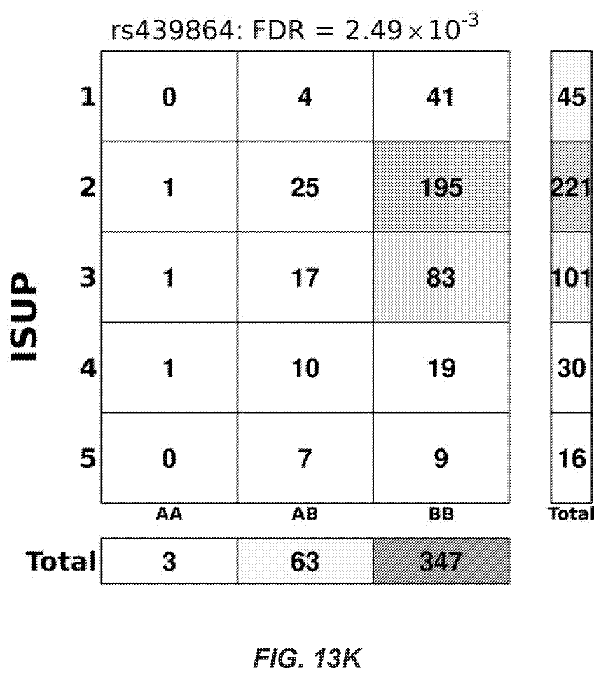

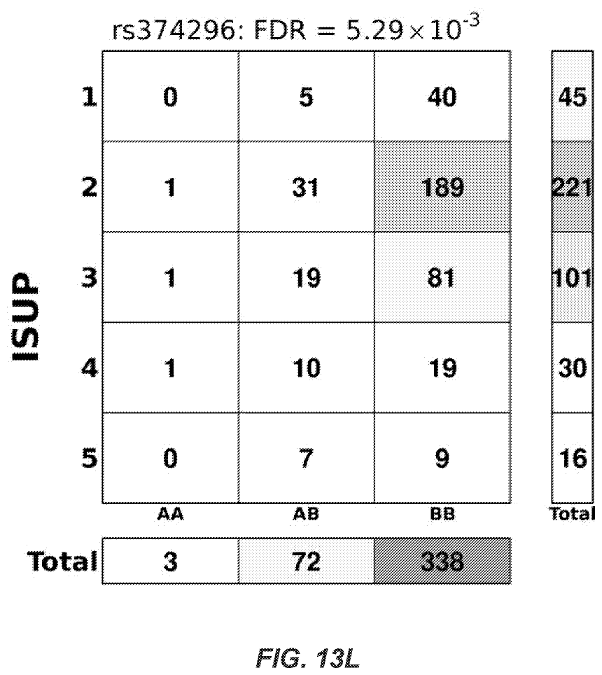

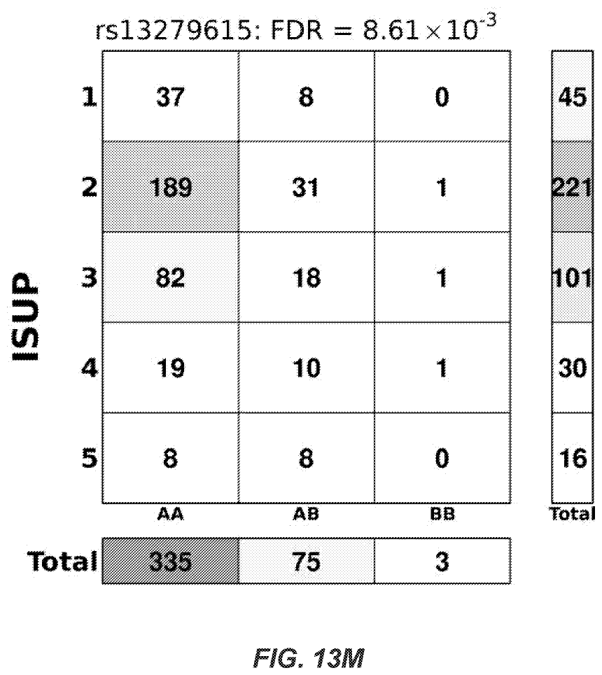

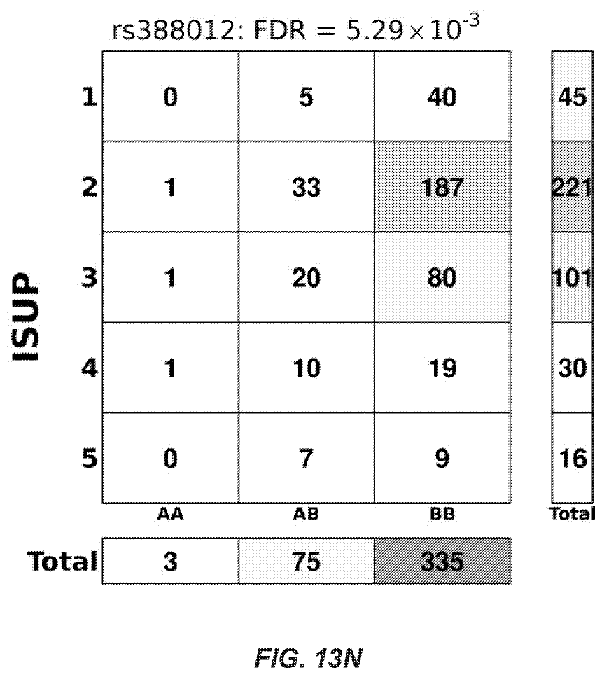

It has been well established that genetic ancestry influences the somatic landscape of prostate cancer^{12-15,59}, but it is unknown if specific germline SNPs contribute a significant proportion of these differences. The inventors quantified the differences in SNP allele frequencies (VAF) between individuals of European, African and East Asian ancestries for dQTL tag SNPs (FIG. 12C; Table 4). Of the 43 dQTL tag SNPs, 42 had significantly different VAF between European and African or between European and East Asian populations, and 30 in both (FDR<0.01; FIGS. 13A-13B). By contrast, only two dQTLs tag SNPs had significantly different VAFs within European populations demonstrating dQTLs are not driven by population stratification (FIG. 13C).

The inventors then focused on SNPs associated with two mutations with strong ancestry differences: T2E and FOXA1^{12-15,59} (FIG. 12C). The T2E gene fusion is less common in individuals of African and East Asian ancestry¹²⁻¹⁵. The inventors considered the rs11203152 dQTL, which was associated with an increased risk of loss of TMPRSS2 in both discovery and both replication cohorts (FIGS. 2D-2E). Concordant with these ancestry trends, the VAF for this SNP was significantly lower in both African and East Asian populations compared to European (VAF_{African}=0.066; VAF_{East Asian}=0.000; VAF_{European}=0.103; FDR<0.01). The inventors then tested the association of rs11203152 with loss of TMPRSS2 in 115 African men with prostate cancer, with a concordant OR despite the small sample-size (OR_{African}=2.45; P_{African}=0.13; FIG. 13D).

FOXA1 SNVs are more common in men of African ancestry than in men of European ancestry¹⁴, while in men of East Asian ancestry a coding hotspot SNV occurs not found in other ancestries⁵⁹. The rs848048 dQTL tag SNP was associated with risk of SNVs in FOXA1 UTR (FIGS. 4D-4E). Again, concordant with these ancestry differences the tag SNP has significantly lower VAF in African populations than in European or Asian ones (VAF_{African}=0.231; VAF_{European}=0.485; VAF_{East Asian}=0.462; OR=0.36; FDR<0.1). The inventors tested the association between rs848048 and SNVs in FOXA1 UTR in 183 African men. The allele distribution was substantially different in African individual compared to European individuals and the association did not replicate in the African cohort (OR_{African}=0.96; P_{african}=1.00) supportive of a germline role in ancestry-related somatic differences (FIG. 13E). Assuming these dQTLs have a similar mechanism across ancestry populations, the inventors estimate that 17.2-31.3% of the ancestral differences in T2E and 23.4-38.4% of the ancestral differences in FOXA1 are potentially explained by these individual dQTLs, suggesting that dQTLs offer a novel way of understanding at least a subset of ancestral differences in cancer genomic landscapes⁶⁰.

dQTLs are Associated with Clinical Outcome

Given that many somatic mutations and mutational processes are predictive of prostate cancer aggression^{35,61} and no prognostic PRS are published to-date, the inventors evaluated whether dQTLs might predict specific clinical features (FIG. 12C). Four dQTLs were nominally associated with biochemical relapse, defined by rising serum PSA levels following primary treatment, and a surrogate for prostate-cancer specific mortality⁶² (P<0.05; FIG. 13D; Table 4). These included two SNPs associated with T2E, rs2837396 and rs2839469 (HR=0.55 & 0.57; P=0.024 & 0.017, respectively; FIGS. 13E-13F) and one SNP, rs5759167, associated with clonal loss of TMPRSS2 (HR=1.85; P=0.017; FIG. 13G), which is surprising given the lack of prognostic value of TMPRSS2-ERG fusions themselves^{63,64}. The remaining SNP, rs12824766, was associated with subclonal loss of CDKN1B (HR=0.45; P=0.030; FIG. 13H). Additionally, four dQTLs associated with subclonal gain of NCOA2 were associated with ISUP grade group at diagnosis (FDR<1.93×10⁻²; FIGS. 13I-13L). By contrast, non-PRS dQTLs were not more likely than chance to be risk-SNPs based on GWAS summary statistics⁹ (P>0.69; permutation test; FIG. 13M).

dQTL Discovery p-Value Distribution is Significantly Skewed

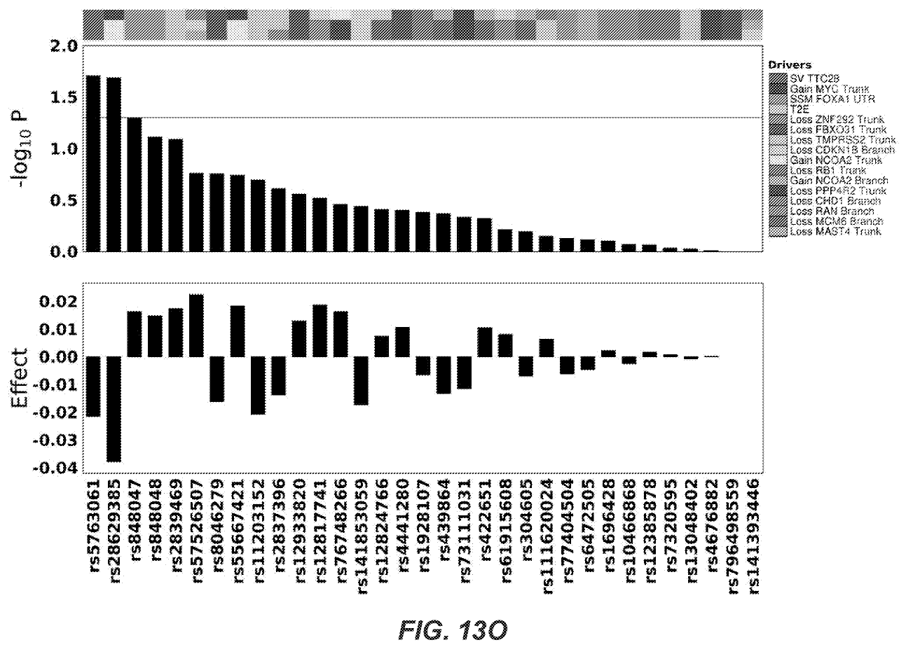

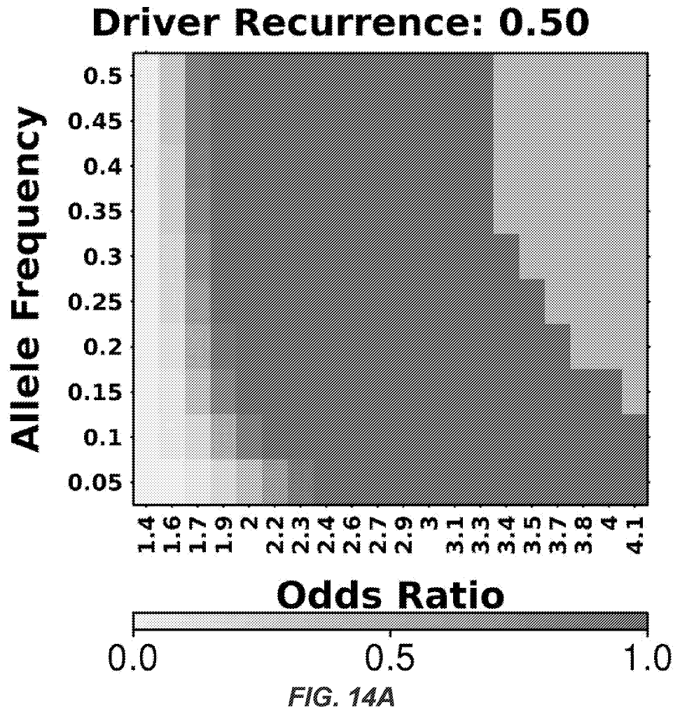

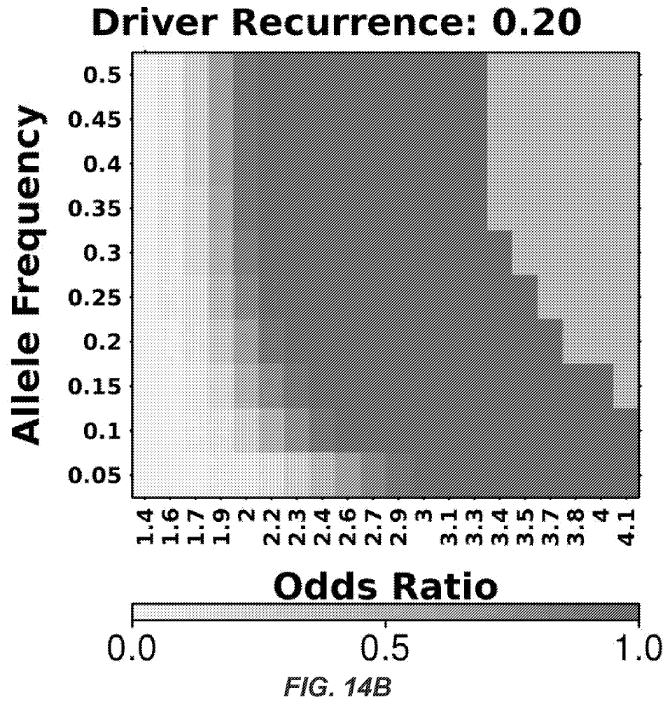

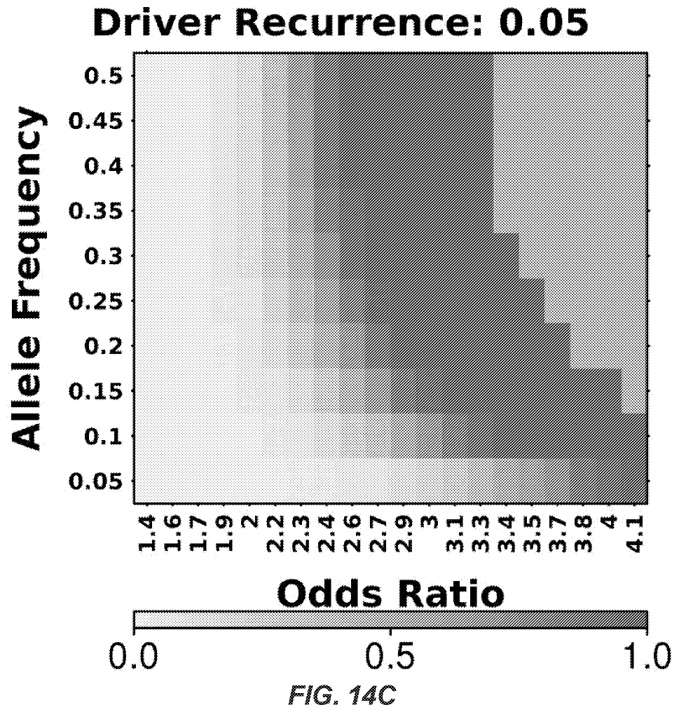

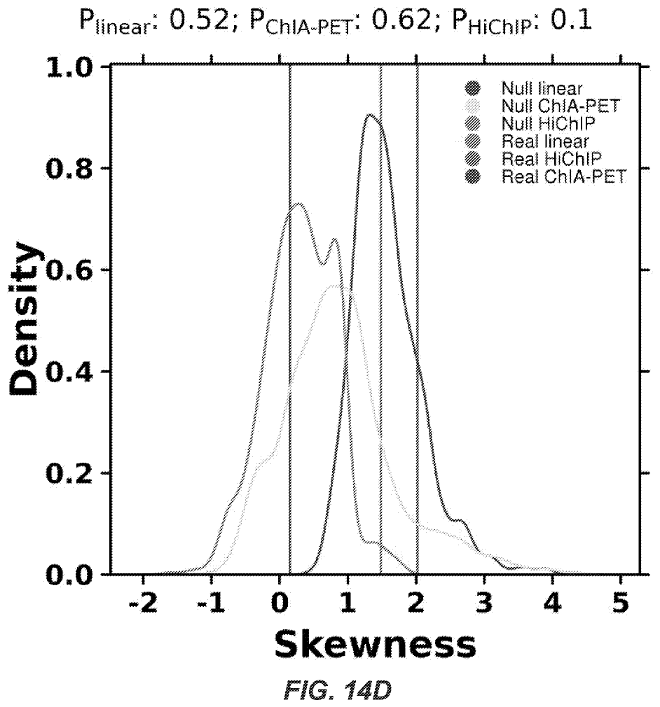

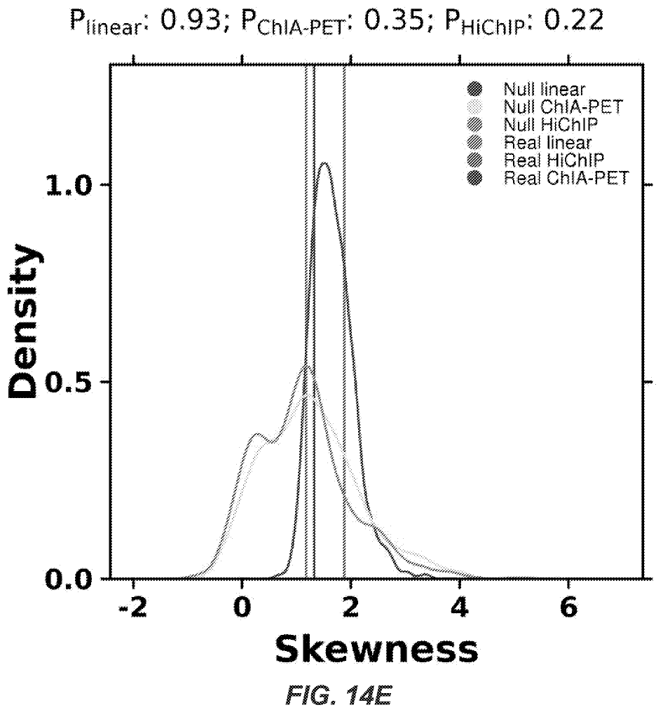

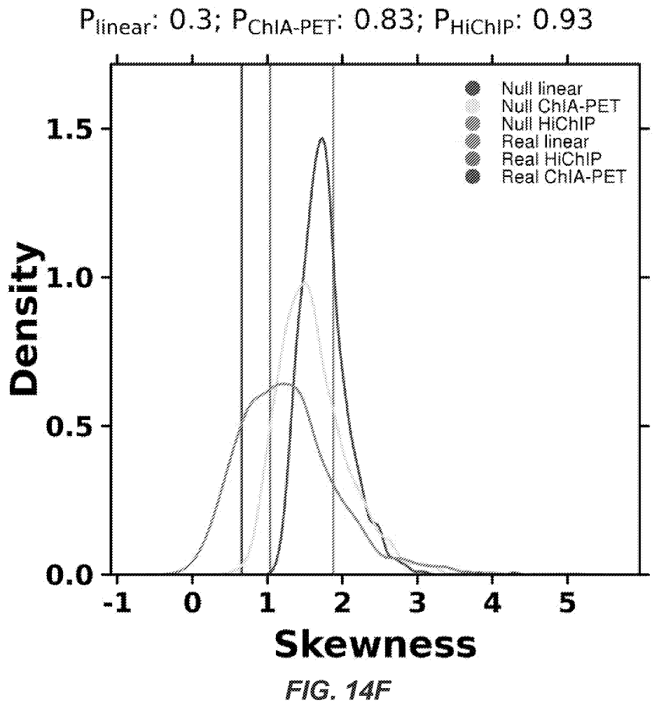

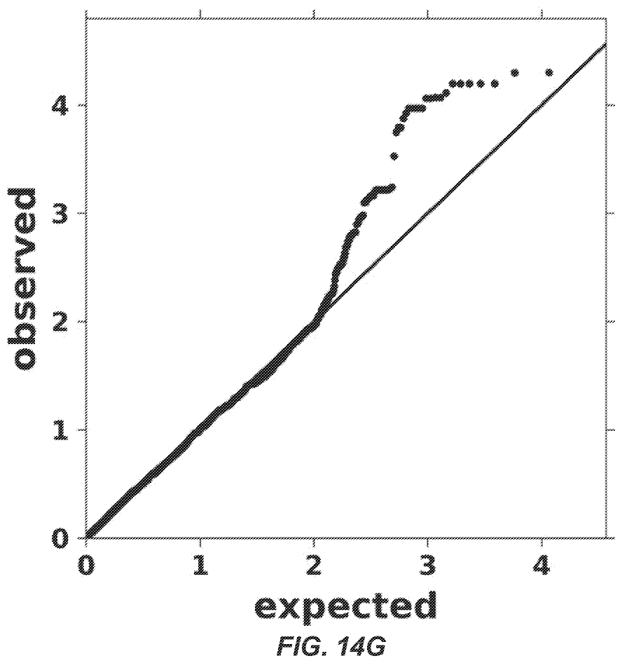

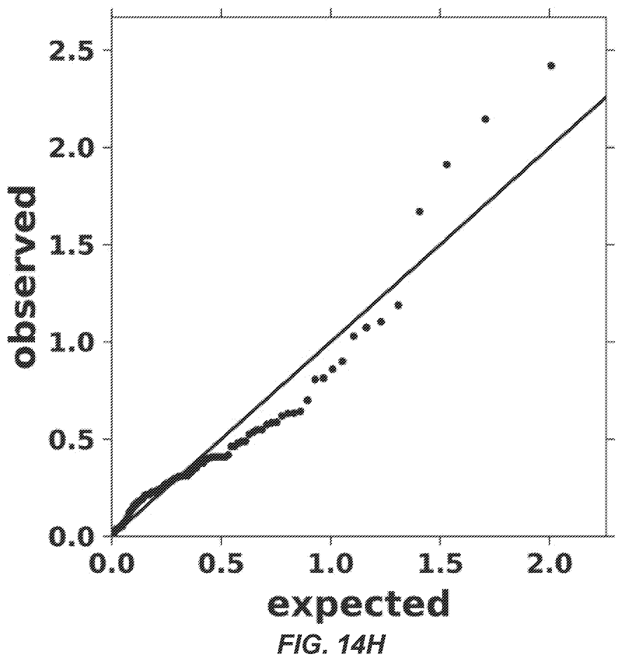

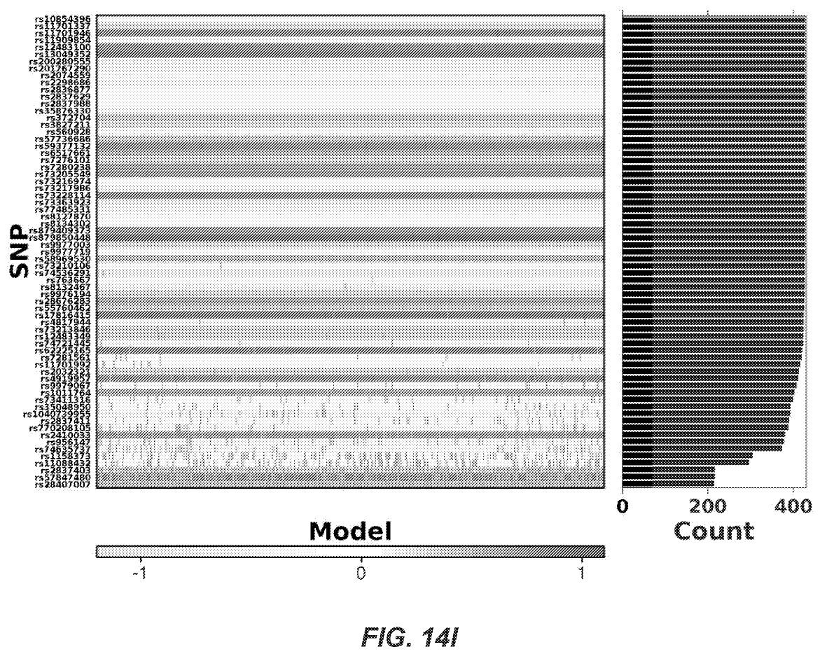

dQTL detection requires matched blood and tumor tissue profiling and thus, despite the presented cohort being the largest whole-genome sequenced prostate cancer cohort available, its much smaller than modern GWAS cohorts. The low frequency of most prostate cancer somatic drivers (˜5-20%) further reduces the power of the inventors' analysis. A cohort of the inventors' size would have 80% power to identify local dQTLs with MAF≥0.4 and OR≥1.7 for somatic drivers present in half the population (P<5×10⁻⁴; FIG. 14A). For typical 5-20% recurrent somatic drivers, the inventors have 80% power to detect OR above 2.0 (FIGS. 14B and 14C). The inventors identified 62 dQTLs involving 20 somatic drivers and 43 SNPs (FIG. 5A). From these figures, the inventors estimate that least 216 additional dQTLs remain to be discovered in larger cohorts at similar effect-sizes (see Methods). Identifying dQTLs genome-wide requires a more stringent p-value threshold (P<5×10⁻⁸) and the inventors were unable to identify dQTLs genome-wide with the inventors' current cohort size.

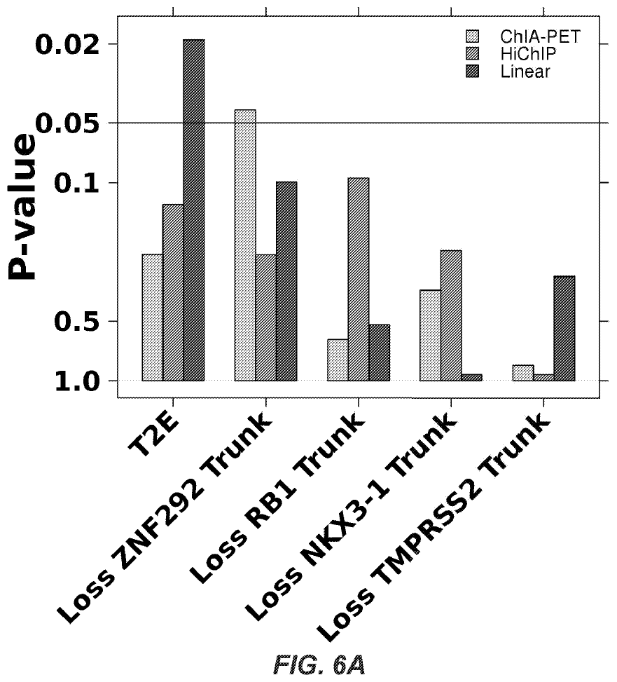

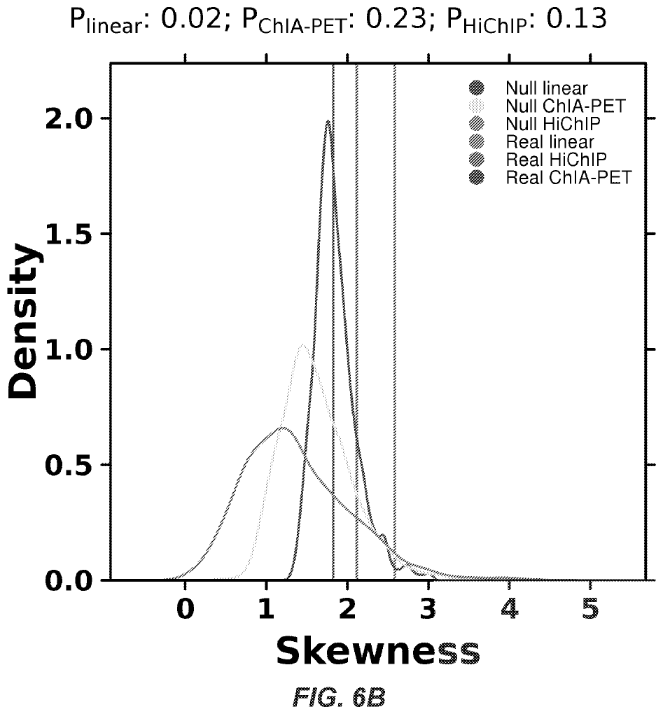

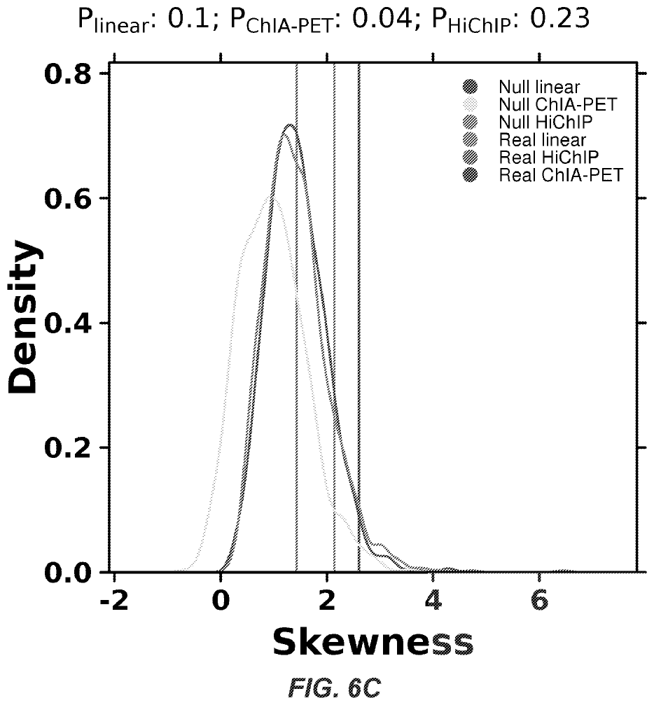

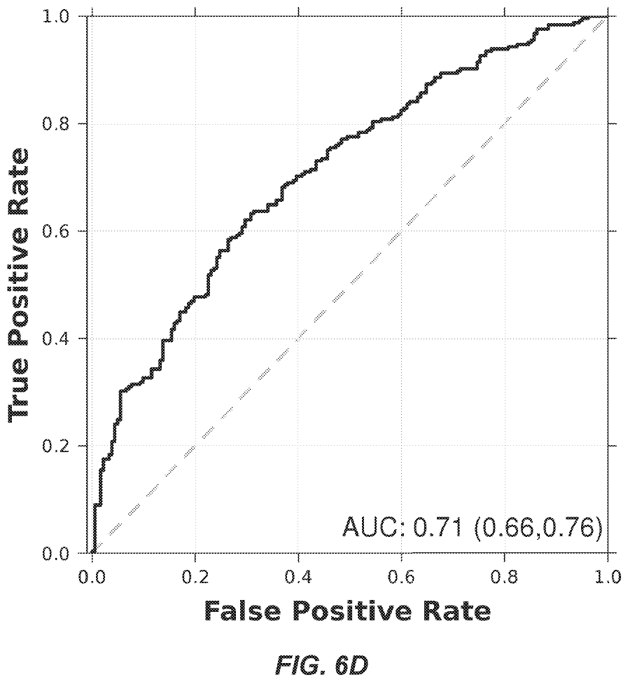

Given this large number of potentially undetected dQTLs, the inventors evaluated whether there was evidence for a large landscape of subthreshold candidate dQTLs. The inventors evaluated the five most recurrent somatic drivers: T2E, clonal loss of ZNF292, clonal loss of RB1, clonal loss of NKX3-1 and clonal loss within TMPRSS2. For each of these, the inventors evaluated the distribution of p-values for the linear, spatial and enhancer local dQTL analyses to evaluate if there were more subthreshold p-values than expected by chance. The inventors compared the skew of the real p-value distributions to empirical null distributions generated by randomly shuffling the assignment of patients with the somatic driver, maintaining the driver frequency (FIGS. 6A-6C; FIGS. 14D-14F). Both T2E and clonal loss of ZNF292 had significantly skewed p-value distributions (FIG. 6A). T2E showed a significant skew towards small p-values in linear local dQTL discovery (FC_{skew vs null skew}=1.43; P=0.019; FIG. 6B; FIG. 14G), while clonal loss of ZNF292 showed a significant skew towards small p-values in spatial local dQTL discovery (P=0.043; FIG. 6C; FIG. 14H). These data suggest many additional prostate dQTLs remain to be identified.

dQTLs can Predict Presence of Somatic Drivers

Given that germline SNPs are present at birth, the presence of dQTLs suggests that they can be used to make predictions about whether a specific somatic driver will occur in a specific man's prostate tumor decades prior to diagnosis. To support this concept, the inventors focused on T2E due to its high recurrence rate of 43% and estimated its local heritability—local heritability gave more stable estimates than genome-wide heritability with the current cohort size. Both genome-based restricted maximum likelihood and Haseman-Elston regression estimated the local heritability of T2E as 7.80-8.23%. Thus, germline genetics explain ˜8% of the variability observed in the formation of T2E. To operationalize this signal, the inventors constructed a driver polygenic risk score (dPRS) to predict the presence of a T2E event. As cohort sizes increase, dPRS scores can be extended to less recurrent and prognostic somatic drivers such as loss of PTEN or gain of MYC^{65,66}. The inventors evaluated prediction accuracy of the dPRS using leave-one-out cross validation (LOOCV). Briefly, the inventors tested the association of all local SNPs, i.e. linear, spatial and enhancer, with T2E in all but one sample. After evaluating multiple p-value thresholds, the inventors included SNPs with P<0.03 and pruned for LD⁶⁷. The inventors tested the resulting dPRS on the one held out sample and repeated this approach until every sample was tested as the held-out sample. Based on the held-out samples, the inventors predicted T2E with area under the receiver operating curve (AUC)=0.71 (95% CI=0.66-0.76; FIG. 6D). One-hundred forty-eight SNPs were included in at least one of the 427 LOOCV dPRS and 34 were included in every dPRS (FIG. 14I).

### Methods

### Discovery Patient Cohort

The discovery patient cohort was comprised of 427 patients with pathologically confirmed prostate cancer and were hormone naive at time of therapy. All patients underwent image-guided external beam radiotherapy (IGRT) or radical prostatectomy (RadP) with curative intent. Two-hundred seventy-six were published and processed as previously described²⁶. Eighty-three patients were previously published in Wedge et al.²⁵, 50 in Baca et al.²², seven in Berger et al.²³ and eleven in Weischenfeldt et al.²⁴. All men were genetically of European descent.

### Whole-Genome Sequencing of Discovery Cohort

For each patient, both blood and tumor sample underwent whole genome sequencing as previously described²⁶. FASTQ files were retrieved for each sample and processed consistently. Raw sequencing reads were aligned to the human reference genome, hs37d5, using BWA-mem⁷⁰ (v0.7.12-0.7.15) at the lane level. Lane level bam files were merged across libraries with duplicates marked within libraries using Picard (v1.121-2.8.2). Local realignment and base quality recalibration was completed on tumor/normal pairs together with the Genome Analysis Toolkit⁷¹ (GATK v3.4.0-3.7.0). Tumor and normal samples were extracted separately, headers corrected (Samtools v0.1.9-1.5)⁷² and files indexed (Picard v2.17.11) into individual sample-level BAMs. Finally, sequencing coverage was computed using picard (v2.17.11) CollectRawWgsMetrics with the default cut-off.

### Germline SNP Detection in Discovery Cohort

Germline SNPs were first identified using GATK (v3.4.0-3.7.0) for each patient individually using HaplotypeCaller followed by VariantRecalibration and ApplyRecalibration⁷¹. Individual VCFs were merged using bcftools (v.1.8) assuming SNPs not present in an individual VCF were homozygous reference. The minor allele frequency (MAF) in the discovery cohort of all SNPs within the merged VCF was calculated and filtered to consider only SNPs with MAF>0.01 (n=10,058,344). Next, all patients were re-genotyped using GATK (v.4.0.2.1) at these sites to produce gVCFs (i.e. with option -ERC GVCF). Individual gVCFs were merged using GenomicsDBImport and joint genotyping was run using GenotypeGVCFs. Finally SNPs were recalibrated using VariantRecalibrator and ApplyVQSR.

### Somatic Variant Detection in Discovery Cohort

Somatic variants were detected as previously described²⁶. Briefly, somatic single nucleotide variants (SNVs) were detected with SomaticSniper (v1.0.5) with mapping quality threshold set to 1 and default parameter⁷³. SNVs were filtered using LOH, read count and high confidence filters provided with the SomaticSniper package. SNVs were further filtered using in-house filters to account for read coverage, germline contamination, mappability, among others. A full description of these filters can be found here²⁶. Somatic copy number alterations (CNAs) were identified using Battenberg (cgpBattenberg v3.3.0, BattenBerg R-core v2.2.8, alleleCount v4.0.1, PCAP-core v4.3.2, cgpVcf v2.2.1, impute2 v2.3.3)⁷⁴. Clonal (i.e. trunk) and subclonal (i.e. branch) CNAs were predicted using the default cut-off of p-value 0.05 and segments length below 10 kb were filtered out. Somatic structural variants (SVs), more specifically inversions and inter-chromosomal translocations, were detected using Delly (v0.7.7-0.7.8) considering a minimum median mapping quality of 20 and a paired-end and split-read cut-off of five⁷⁵. Germline SVs were filtered out by considering a consolidated list of structural variants from the blood reference samples in this cohort. SVs were annotated to genes using SnpEff (v4.3R) on a bed file of breakpoints⁷⁶.

### Recurrent Somatic Drivers in Prostate Cancer

The inventors considered a set of 180 somatic drivers were identified in 666 localized prostate tumors²⁶, and included those with a frequency ≥5% in the discovery cohort. This resulted in analysis of 37 somatic drivers: 24 CNA losses (14 trunk and 10 branch), 3 CNA gains (2 trunk and 1 branch), 7 SVs including the recurrent T2E fusion between TMPRSS2 and ERG and 3 SNVs.

dQTL Discovery: Risk SNPs dQTLs

The 147 SNP polygenic risk score generated by Schumacher et al.⁹ was first considered for dQTL discovery. Of the 147 SNPs, 135 had a MAF>0.05 in the discovery cohort. All 135 SNPs were tested for association with all 37 somatic drivers using a logistic regression model correcting for the first two genetic principal components to adjust for population stratification. P-values were adjusted for multiple-hypothesis testing using the Benjamini & Hochberg false discovery correction. Significance was defined as FDR<0.1. In addition to testing individual SNPs, a polygenic risk score, as described in Schumacher et al., was calculated for each patient based on the dosage of each of the 147 SNPs and the reported betas⁹. The inventors calculated the association between PRS and PGA or number of drivers using a Mann-Whitney test and a Spearman correlation.

dQTL Discovery: Linear Local dQTLs

Local dQTLs was first defined based on the linear orientation of the genome. Considering each somatic event could be defined by a single gene, germline SNPs within ±500 kbp of the affected gene were interrogated for their association with the somatic event. Associations were quantified using a logistic regression model correcting for the first two genetic principal components and the somatic mutation burden (i.e. PGA when testing CNAs, SNV mutation rate when testing SNVs and SV count when testing SVs). Haplotype blocks within the defined linear local region were calculated considering the definition by Gabriel et al.⁷⁷ and a Bonferroni threshold considering α=0.1 was used to determine significance.

dQTL Discovery: Spatial Local dQTLs

Next, local dQTLs were defined taking into consideration the three-dimensional structure of DNA. The term spatial local was defined as regions of the DNA, outside ±500 kbp around the affected gene, that loop to interact with the driver gene. First, these regions were defined by RAD21 and RNA polymerase II ChIA-PET profiling in LNCaP, DU145, VCaP and RWPE1 cell lines³⁷. Coordinates of driver genes were overlapped with peak anchor regions using Bedtools. Based on an interaction map, peak anchors paired with driver-gene-overlapped peaks were defined as interacting regions. Similar to linear local dQTLs, associations were quantified using a logistic regression model correcting for the first two genetic principal components and the somatic mutation burden. Again, haplotype blocks within the defined spatial local region were calculated considering the definition by Gabriel et al.⁷⁷ and a Bonferroni threshold considering α=0.1 was used to determine significance.

dQTL Discovery: Enhancer Local dQTLs

Next, the inventors defined spatial local regions based on HiChIP H3K27ac profiling in LNCaP cell lines. HiChIP was conducted as reported previously. Again, associations were quantified using a logistic regression model correcting for the first two genetic principal components and the somatic mutation burden and haplotype blocks within the defined enhancer local region were calculated considering the definition by Gabriel et al.⁷⁷ and a Bonferroni threshold considering α=0.1 was used to determine significance.

### Prostate Cancer Replication Cohort

Individuals of European descent, as determined by Yuan et al.⁶⁰, from TCGA PRAD project were used as a replication cohort²⁹. As described previously²⁰, concordance between SNP6 microarray (SNP6) genotypes and whole exome sequencing (WXS) of blood sample calls was evaluated and only samples with >80% concordance were retained (412 samples). Genotypes were imputed using the Michigan Imputation Server—pre-phasing using Eagle (v2.4)⁷⁸, imputation using Minimac4⁷⁹ and the Haplotype Reference Consortium (release 1.1) panel⁸⁰. A final list of 40,401,582 SNPs were then available for validation studies. A second cohort of 140 Australian men with localized prostate cancer was used to supplement the replication cohort. All patients had blood and tumor WGS that was processed with the same pipelines as the discovery cohort, including evolutionary timing of CNAs²⁶. Similar to the discovery cohort, germline SNPs were identified using GATK (v3.4.0-3.7.0)⁷¹. First, HaplotypeCaller was run on the normal and tumor BAMs together, followed by Variant Recalibration and ApplyRecalibration, following GATK best practices. Germline SNPs were filtered for somatic and ambiguous variants that had more than one alternate base.

### Pan-Cancer Replication Cohort

The inventors leveraged the Pan-cancer Analysis of Whole Genomes (PCAWG)³⁶ to test the replication of dQTLs in other cancer types, using germline VCFs and somatic CNA calls from the Pan-Cancer Analysis of Whole Genomes from DCC (available on the World Wide Web at dcc.icgc.org/releases/PCAWG/). The inventors considered only adult cancers with >100 samples: breast, ovarian, pancreatic and liver cancer. Next, the inventors only considered patients of European ancestry which resulted in 134 breast, 91 ovarian, 116 pancreatic and 0 liver cancer patients. Thus, the inventors did not consider liver cancer in replication analysis. The inventors tested somatic events with a recurrence rate ≥5% in each cancer type.

Replication of dQTLs

dQTLs with available somatic profiling and germline genotyping were tested in the replication cohort. Because TCGA does not have WGS, dQTLs involving SVs could not be tested and the evolutionary timing of CNAs could not be determined in these patients. Thus, dQTLs involving CNAs were tested in TCGA without considering trunk vs branch classifications. dQTLs in all replication cohorts were tested using the same logistic regression model as used in discovery, correcting for the first two genetic principal components and the total burden of somatic mutation type being tested (i.e. PGA, SNV mutation rate or SV count). dQTLs were considered to have replicated if FDR<0.1 and sign(log₂(OR_{discovery}))=sign(log₂(OR_{replication})).

Replication of dQTLs in ICGC EOPC-DE

The inventors identified 16 dQTLs that were associated with somatic events with a recurrence rate ≥5% in the EOPC-DE cohort and had concordant ORs in the discovery and replication cohorts. The candidate SNPs were studied across 238 prostate cancer patients with European ancestry from the ICGC EOPC-DE cohort³⁸. Germline SNP genotyping and quality control was performed as previously described⁸¹. Association between germline SNP genotypes, age at diagnosis, and presence of somatic mutation phenotypes was performed using logistic regression models in python (stats package version 0.11.1). Likelihood ratio tests were used to compare an age model (phenotype ˜age) with an additive genetic model that included both age and SNP genotypes (phenotype ˜age+SNP).

### Heritability of Somatic Drivers

The inventors estimated heritability for T2E by combining both the discovery and replication cohorts. SNPs that matched the following criteria were filtered out using plink (v.19): on a sex chromosome, HTHE INVENTORS P<1×10⁻⁶ and genotype missing in more than 5% of samples. Samples were also filtered for relatedness using a 0.50 threshold for inclusion. The inventors quantified local heritability so only linear local and spatial local SNPs, as described above, were included. The inventors used two methods to estimate heritability to ensure the estimate was stable: Restricted maximum likelihood (REML) and Haseman-Elston (HE) regression. Both methods are implemented in GCTA (v1.93.0)⁸². The following covariates were included in the model: the first five genetic principal components, percent of the somatic genome altered by CNA and a variable indicating if genotype was from WGS or SNP6+WXS followed by imputation. For both approaches, the inventors first calculated segment-based LD scores (200 kbp segments) and stratified SNPs into four groups based on LD score⁸³. The inventors computed GRMs using the stratified SNPs and performed REML and HE using the multiple GRMs.

Germline Methylation (meQTL) Associations

To assess the effect of the 62 dQTLs on the tumor methylome, the 43 unique SNPs were evaluated for local meQTLs, defined as probes ±10 kbp around the SNP, using a linear regression model correcting for the first two genetic principal components. P-values were adjusted for multiple-hypothesis testing using the Benjamini & Hochberg false discovery correction. Significance was defined as FDR<0.05. Significant meQTLs were next replicated in the TCGA cohort using the same linear regression modeling. Here replication was defined as FDR_{replication}<0.05 and sign(β_{replication})=sign(β_{discovery}). Finally, replicated meQTLs were tested for tumor specificity considering patients that had matched tumor/reference methylation profiling (n=50). Tumor specificity was defined as FDRₜᵤₘₒᵣ<0.05 and FDR_{reference}>0.05 or sign(βₜᵤₘₒᵣ)≠sign(β_{reference}) using the same linear regression model.

### Germline-Chromatin Associations

Peak bed files for H3K27ac (n=92), H3K27me3 (n=76), AR (n=88) and H3K4me3 (n=56) were downloaded for an independent cohort of 94 localized prostate cancer patients from the Gene Expression Omnibus (GSE120738)³⁹. dQTLs overlapping each target were identified using the downloaded bed files. The inventors considered a dQTL overlapping if any of the SNPs in its haplotype block overlapped the target. A second cohort of 48 localized prostate cancer patients was additionally profiled, as described previously²⁰. Briefly, both adenocarcinoma and non-malignant prostate tissue from each patient was subjected to ChIP-Seq for H3k27ac (N=48), H3k4me2 (N=6), H3k4me3 (N=4), FOXA1 (N=10) and HOXB13 (N=9) and blood samples were genotyped for germline SNPs followed by imputation using the HRC panel⁸⁰. Sites of allelic imbalance in the ChIP-Seq peaks were identified by first correcting for mapping bias using the WASP pipelines⁸⁴, peak calling using MACS2 and finally testing for allele-specific signal using GATK ASEReadCounter⁷¹ and a beta-binomial test. Each test was performed once for samples from normal, tumor, or both, as well as a test for difference in imbalance between tumor and normal. Peaks were considered “imbalanced” in each of these four test categories if any of the SNPs tested for that peak exhibited allele-specific signal at a 5% FDR. Finally, the inventors tested the overlap of dQTLs with published ChIP-Seq data from LNCaP, PC3, 22Rv1, VCaP and RWPE-1 cell lines⁴⁰⁻⁵³. If multiple target:treatment pairs existed the median number of overlapping SNPs was used.

Germline-RNA (eQTL) and Germline-Protein (pQTL) Associations

Next, the 43 SNPs involved in the 62 dQTLs were tested for their effect on the transcriptome. The inventors evaluated local eQTLs, defined as genes ±1 Mbp around the SNP. mRNA abundance TPM values for each gene were rank inverse normalized. eQTLs were tested using a linear regression model correcting for the first two genetic principal components and ten PEER⁸⁵ factors to adjust for noise in the RNA-Seq data. P-values were adjusted for multiple-hypothesis testing using the Benjamini & Hochberg false discovery correction. Nominally significant eQTLs were considered for pQTL discovery using protein abundances from mass spectrometry as described previously⁸⁶. pQTLs were tested using a linear regression model correcting for the first two genetic principal components and ten PEER factors to adjust for noise in the mass spectrometry data.

### Germline-Clinical Associations

Germline SNPs in dQTLs were associated with clinical characteristics including PSA, ISUP Grade Group, T-category, age at diagnosis and biochemical recurrence. PSA and age were tested using linear regression model, correcting for the first two genetic principal components. ISUP and t-category were tested by using an ordinal linear regression model, correcting for the first two genetic principal components. Survival analysis with biochemical recurrence was tested using a Cox Proportional Hazards model. Three genetic models, dominant, recessive and co-dominant, were tested and the model with the lowest AIC was reported. Kaplan-Meier curves were plotted and HR adjusted for primary treatment.

### Somatic SNV Enrichment

For each of the 40 unique SNPs involved in the 59 dQTLs, excluding 3 dQTLs associated with SNVs within the 3′ UTR of FOXA1, the inventors calculated the number of somatic SNVs within ±10 kbp. To assess if the number of co-occurring somatic SNVs was more than expected by chance, the inventors ran a permutation analyses were the inventors randomly sampled 40 SNPs from all local SNPs that were not dQTLs and calculated the number of somatic SNVs within ±10 kbp. The inventors ran this permutation 10,000 times to generate a null distribution. A p-value was calculated as the number of iterations whose median number of co-occurring somatic SNVs≥the real number of co-occurring somatic SNVs divided by the number of iterations.

### Ancestral Variant Allele Frequency Bias

Variant allele frequencies in European (n=7,718), African (n=4,359) and East Asian (n=780) populations for the 43 dQTL SNPs were extract from gnomAD (v2.1.1)⁸⁷. Allele frequencies in African and East Asian populations were compared to European population using Fisher's Exact test. As a control, North-West European VAFs were compared again Other Non-Finnish European VAFs using Fisher's Exact test. These two European populations were chosen because they had the largest sample number in gnomAD. To estimate the proportion of ancestral differences in T2E and FOXA1 mutation frequency explained by dQTLs, the inventors compared the ORs of ancestry-somatic associations and dQTLs ORs multiplied by normalized variant allele frequency differences between the two ancestry groups. For example:

$$\frac{{OR}_{dQTL} x \frac{{VAF}_{European} - {VAF}_{African}}{{VAF}_{European}}}{{OR}_{EuropeanvsAfrican}}$$

The inventors estimated OR_{European vs. African} (T2E)=5.00 and OR_{European vs. African} (FOXA1 SNVs)=0.50 based on Huang et al.¹³ and Lindquist et al.¹⁴ compared to the somatic driver frequency in the discovery cohort. The inventors estimated OR_{European vs. East Asian} (T2E)=7.47 and OR_{European vs. East Asian} (FOXA1 SNVs)=0.07 based on Li et al.⁵⁹ compared to the somatic driver frequency in the discovery cohort.

dQTL Power Analysis

Power was estimated based on the non-centrality parameter of the χ² statistic under the alternative hypothesis using the R package gwas-power (available on the World Wide Web at github.com/kaustubhad/gwas-power). Power was calculated for varying MAF and effect size values considering sample sizes reflective of somatic driver frequencies 0.05, 0.20 and 0.50 in the discovery cohort. To estimate the number of non-detected dQTLs, discovered dQTLs were binned based on their MAF, effect size and somatic driver frequency and the number of detected dQTLs in each bin was divided by the corresponding power to estimate the total number of dQTLs expected. Next, the inventors subtracted the number of discovered dQTLs from the total number of dQTLs to estimate the number of non-detected dQTLs.

Assessment of Skew of dQTL p-Value Distributions

To determine if dQTL p-value distributions were significantly skewed to small p-values more than expected by chance alone, a null distribution for each analysis (i.e. linear local and spatial local) and each somatic driver was generated by permuting the somatic driver labels. That is, for a single somatic event, patients were randomly assigned whether or not they had the somatic event while maintaining the true frequency of the event in the cohort. Next, both linear and spatial local dQTL discovery was conducted as described above with the permuted somatic driver labels. The skew of the −log₁₀ p-value distribution was calculated and compared to the true distribution. P-values were calculated by considering the number of permutation iterations that had skew>real skew divided by the number of iterations performed. One thousand iterations were performed for each somatic driver.

### Driver Polygenic Risk Score Generation

dPRS to predict the presence of T2E were built using the LD-pruning and thresholding method implemented in Ldpred⁶⁷ and considering only local SNPs taking the union of the linear, spatial and enhancer local definitions described earlier. To assess the accuracy of the model, the inventors used leave-one-cross validation. The inventors generated association statistics considering all but one sample and then ran ldpred p+t before testing the resulting dPRS on the left out sample. This process was replicated for every sample. Association statistics were calculated with logistic regression model correcting for the first two genetic principal components and the somatic mutation burden. The inventors considered p-values thresholds 1, 0.03, 0.01, 0.003, 0.001, 0.0003, 0.0001 and generated receiver operating curves based on the left-out sample predictions to access the accuracy of each model.

### Data Visualization

Visualizations were generated in the R statistical environment (v3.3.1) with the lattice (v0.24-30), latticeExtra (v0.6-28) and BPG (v5.6.23) packages⁸⁸.

### Data Availability

Raw sequencing data are available in the European Genome-phenome Archive under accession EGAS00001000900 (https://www.ebi.ac.uk/ega/studies/EGAS00001000900). Processed variant calls are available through the ICGC Data Portal under the project PRAD-CA (https://dcc.icgc.org/projects/PRAD-CA). Methylation data are available in the Gene Expression Omnibus under accession GSE84043. TCGA WGS/WXS data are available at Genomic Data Commons Data Portal (available on the World Wide Web at gdc-portal.nci.nih.gov/projects/TCGA-PRAD). Primary samples ChIP-Seq data was retrieved from Gene Expression Omnibus under accession GSE120738.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Vogelstein, B. et al. Cancer genome landscapes. Science 339, 1546-58 (2013).
2. Garraway, L. A. & Lander, E. S. Lessons from the Cancer Genome. Cell 153, 17-37 (2013).
3. Tomasetti, C., Li, L. & Vogelstein, B. Stem cell divisions, somatic mutations, cancer etiology, and cancer prevention. Science 355, 1330-1334 (2017).
4. Mucci, L. A. et al. Familial Risk and Heritability of Cancer Among Twins in Nordic Countries. JAMA 315, 68 (2016).
5. Tomlinson, I. P. et al. A genome-wide association study identifies colorectal cancer susceptibility loci on chromosomes 10p14 and 8q23.3. Nat. Genet. 40, 623-630 (2008).
6. Petersen, G. M. et al. A genome-wide association study identifies pancreatic cancer susceptibility loci on chromosomes 13q22.1, 1932.1 and 5p15.33. Nat. Genet. 42, 224-228 (2010).
7. Michailidou, K. et al. Association analysis identifies 65 new breast cancer risk loci. Nature 551, 92-94 (2017).
8. Noone A M, Howlader N, Krapcho M, Miller D, Brest A, Yu M, Ruhl J, Tatalovich Z, Mariotto A, Lewis D R, Chen H S, Feuer E J, C. K. SEER Cancer Statistics Review, 1975-2015. National Cancer Institute. Bethesda, MD (2018). Available at: https://seer.cancer.gov/csr/1975_2015/. (Accessed: 12 Feb. 2019)
9. Schumacher, F. R. et al. Association analyses of more than 140,000 men identify 63 new prostate cancer susceptibility loci. Nat. Genet. 50, 928-936 (2018).
10. Pritchard, C. C. et al. Inherited DNA-repair gene mutations in men with metastatic prostate cancer. N. Engl. J. Med. 375, 443-453 (2016).
11. Leongamornlert, D. A. et al. Germline DNA Repair Gene Mutations in Young-onset Prostate Cancer Cases in the UK: Evidence for a More Extensive Genetic Panel. Eur. Urol. (2019). doi:10.1016/J.EURURO.2019.01.050
12. Ren, S. et al. Whole-genome and Transcriptome Sequencing of Prostate Cancer Identify New Genetic Alterations Driving Disease Progression. Eur. Urol. 73, 322-339 (2018).
13. Huang, F. W. et al. Exome Sequencing of African-American Prostate Cancer Reveals Loss-of-Function ERF Mutations. Cancer Discov. 7, 973-983 (2017).
14. Lindquist, K. J. et al. Mutational landscape of aggressive prostate tumors in African American men. Cancer Res. 76, 1860-1868 (2016).
15. Blackburn, J. et al. TMPRSS2-ERG fusions linked to prostate cancer racial health disparities: A focus on Africa. Prostate 79, 1191-1196 (2019).
16. Taylor, R. A. et al. Germline BRCA2 mutations drive prostate cancers with distinct evolutionary trajectories. Nat. Commun. 8, 13671 (2017).
17. Briollais, L. et al. Germline Mutations in the Kallikrein 6 Region and Predisposition for Aggressive Prostate Cancer. JNCI J. Natl. Cancer Inst. 109, (2017).
18. Romanel, A. et al. Inherited determinants of early recurrent somatic mutations in prostate cancer. Nat. Commun. 8, 48 (2017).
19. Heyn, H. et al. Linkage of DNA Methylation Quantitative Trait Loci to Human Cancer Risk. Cell Rep. 7, 331-338 (2014).
20. Houlahan, K. E. et al. Genome-wide germline correlates of the epigenetic landscape of prostate cancer. Nat. Med. 25, 1615-1626 (2019).
21. Knudson, A. G. Mutation and Cancer: Statistical Study of Retinoblastoma. 68, 820-823 (1971).
22. Baca, S. C. et al. Punctuated Evolution of Prostate Cancer Genomes. Cell 153, 666-677 (2013).
23. Berger, M. F. et al. The genomic complexity of primary human prostate cancer. Nature 470, 214-220 (2011).
24. Weischenfeldt, J. et al. Integrative Genomic Analyses Reveal an Androgen-Driven Somatic Alteration Landscape in Early-Onset Prostate Cancer. Cancer Cell 23, 159-170 (2013).
25. Wedge, D. C. et al. Sequencing of prostate cancers identifies new cancer genes, routes of progression and drug targets. Nat. Genet. 50, 682-692 (2018).
26. Yamaguchi T. N., et al. Molecular and evolutionary origins of prostate cancer grade. Cell (In review).
27. Ewing, A. D. et al. Combining tumor genome simulation with crowdsourcing to benchmark somatic single-nucleotide-variant detection. Nat. Methods 12, (2015).
28. Lee, A. Y. et al. Combining accurate tumor genome simulation with crowdsourcing to benchmark somatic structural variant detection. Genome Biol. 19, 188 (2018).
29. The Cancer Genome Atlas Research Network. The Molecular Taxonomy of Primary Prostate Cancer. Cell 163, 1011-1025 (2015).
30. Luedeke, M. et al. Prostate cancer risk regions at 8q24 and 17q24 are differentially associated with somatic TMPRSS2:ERG fusion status. Hum Mol Genet. 25, 5490-5499 (2016).
31. Chen, W. S. et al. Germline polymorphisms associated with impaired survival outcomes and somatic tumor alterations in advanced prostate cancer. Prostate Cancer Prostatic Dis 23, 316-323 (2020).
32. Ostendorf, B. N. et al. Common germline variants of the human APOE gene modulate melanoma progression and survival. Nat Med (2020).
33. Qing, T. et al. Germline variant burden in cancer genes correlates with age at diagnosis and somatic mutation burden. Nat Commun. 11, 2438 (2020).
34. Penney, K. L. et al. Association of prostate cancer risk variants with TMPRSS2:ERG Status: Evidence for distinct molecular subtypes. Cancer Epidemiol. Biomarkers Prev. 25, 745-749 (2016).
35. Espiritu, S. M. G. et al. The evolutionary landscape of localized prostate cancer drives clinical aggression. Cell 173, 1003-1013 (2018).
36. The ICGC/TCGA Pan-Cancer Analysis of Whole Genomes Consortium. Pan-cancer analysis of whole genomes. Nature 578, 82-93 (2020).
37. Ramanand, S. G. et al. The landscape of RNA polymerase II associated chromatin interactions in prostate cancer. JCI 130, 3987-4005 (2020).
38. Gerhauser, C. et al. Molecular evolution of early-onset prostate cancer identifies molecular risk markers and clinical trajectories. Cancer Cell 34, 996-1011 (2018).
39. Stelloo, S. et al. Integrative epigenetic taxonomy of primary prostate cancer. Nat. Commun. 9, 4900 (2018).
40. Lee, J. K. et al. N-Myc drives neuroendocrine prostate cancer initiated from human prostate epithelial cells. Cancer Cell 29, 536-547 (2016).
41. Yu, J. et al. An integrated network of androgen receptor, polycomb, and TMPRSS2-ERG gene fusions in prostate cancer progression. Cancer Cell 17, 443-54 (2010).
42. Wang, D. et al. Reprogramming transcription by distinct classes of enhancers functionally defined by eRNA. Nature 474, 390-4 (2011).
43. Tan, P. Y. et al. Integration of regulatory networks by NKX3-1 promotes androgen-dependent prostate cancer survival. Mol Cell Biol 32, 399-414 (2012).
44. Hazelett, D. J. et al. Comprehensive functional annotation of 77 prostate cancer risk loci. PLoS Genet 10, e1004102 (2014).
45. Jin, H. J. et al. Cooperativity and equilibrium with FOXA1 define the androgen receptor transcriptional program. Nat Commun 5, 3972 (2014).
46. Xu, K. et al. EZH2 oncogenic activity in castration-resistant prostate cancer cells is Polycomb-independent. Science 338, 1465-9 (2012).
47. Zhang, X. et al. Integrative functional genomics identifies an enhancer looping to the SOX9 gene disrupted by the 17q24.3 prostate cancer risk locus. Genome Res 22, 1437-46 (2012).
48. Chen, Y. et al. ETS factors reprogram the androgen receptor cistrome and prime prostate tumorigenesis in response to PTEN loss. Nat Med 19, 1023-9 (2013).
49. ENCODE Project Consortium. An integrated encyclopedia of DNA elements in the human genome. Nature 489, 57-74 (2012).
50. Liang, Y. et al. LSDI-mediated epigenetic reprogramming drives CENPE expression and prostate cancer progression. Cancer Res 77, 5479-5490 (2017).
51. Sutinen, P. et al. SUMOylation modulates the transcriptional activity of androgen receptor in a target gene and pathway selective manner. Nucleic Acids Res 42, 8310-8319 (2014).
52. Taberlay, P. C. et al. Reconfiguration of nucleosome-depleted regions at distal regulatory elements accompanies DNA methylation of enhancers and insulators in cancer. Genome Res 24, 1421-1432 (2014).
53. Rickman, D. S. et al. Oncogene-mediated alterations in conformation. Proc Natl Acad Sci USA 109, 9083-9088 (2012).
54. Gonzalez-Perez, A. et al. Local determinants of the mutational landscape of the human genome. Cell 177, 101-114 (2019).
55. Pomerantz, M. M. et al. Prostate cancer reactivates developmental epigenomic programs during metastatic progression. Nat Genet (2020).
56. Xu, X. et al. Variants at IRX4 as prostate cancer expression quantitative trait loci. Eur. J. Hum. Genet. 22, 558-563 (2014).
57. GTEx Consortium. Genetic effects on gene expression across human tissues. Nature 550, 204-213 (2017).
58. Mazrooei, P. et al. Cistrome partitioning reveals convergence of somatic mutations and risk variants on master transcription regulators in primary prostate tumors. Cancer Cell 36, 674-689 (2019).
59. Li, J. et al. A genomic and epigenomic atlas of prostate cancer in Asian populations. Nature 580, 93-99 (2020).
60. Yuan, J. et al. Integrated Analysis of Genetic Ancestry and Genomic Alterations across Cancers. Cancer Cell 34, 549-560.e9 (2018).
61. Lalonde, E. et al. tumor genomic and microenvironmental heterogeneity for integrated prediction of 5-year biochemical recurrence of prostate cancer: a retrospective cohort study. Lancet Oncoli 15, 1521-1532 (2014).
62. Jackson, W. C. et al. Intermediate endpoints after postprostatectomy radiotherapy: 5-year distant metastasis to predict overall survival. Eur Urol 74, 413-419 (2018).
63. Gopalan, A. et al. Tmprss2-erg gene fusion is not associated with outcome in patients treated by prostatectomy. Cancer Res. 69, 1400-1406 (2009).
64. Pettersson, A. et al. The TMPRSS2:ERG rearrangement, ERG expression, and prostate cancer outcomes: A cohort study and meta-analysis. Cancer Epidemiol. Biomarkers Prev. 21, 1497-1509 (2012).
65. Fraser, M. et al. Genomic hallmarks of localized, non-indolent prostate cancer. Nature 541, 359-364 (2017).
66. Bhandari, V. et al. Molecular landmarks of tumor hypoxia across cancer types. 51, 308-318 (2019).
67. Vilhjálmsson, B. J. et al. Modeling Linkage Disequilibrium Increases Accuracy of Polygenic Risk Scores. Am. J. Hum. Genet. 97, 576-592 (2015).
68. Carter, H. et al. Interaction Landscape of Inherited Polymorphisms with Somatic Events in Cancer. Cancer Discov. 7, 410-423 (2017).
69. The International HapMap Consortium. A haplotype map of the human genome. Nature 437, 1299-1320 (2005).
70. Li, H. & Durbin, R. Fast and accurate short read alignment with Burrows-Wheeler transform. Bioinformatics 25, 1754-1760 (2009).
71. McKenna, A. et al. The Genome Analysis Toolkit: a MapReduce framework for analyzing next-generation DNA sequencing data. Genome Res. 20, 1297-303 (2010).
72. Li, H. et al. The Sequence Alignment/Map format and SAMtools. Bioinformatics 25, 2078-2079 (2009).
73. Larson, D. E. et al. SomaticSniper: identification of somatic point mutations in whole genome sequencing data. Bioinformatics 28, 311-7 (2012).
74. Nik-Zainal, S. et al. The life history of 21 breast cancers. Cell 149, 994-1007 (2012).
75. Rausch, T. et al. DELLY: structural variant discovery by integrated paired-end and split-read analysis. Bioinformatics 28, i333-i339 (2012).
76. Cingolani, P. et al. A program for annotating and predicting the effects of single nucleotide polymorphisms, SnpEff: SNPs in the genome of Drosophila melanogaster strain w1118; iso-2; iso-3. Fly (Austin). 6, 80-92 (2012).
77. Gabriel, S. B. et al. The structure of haplotype blocks in the human genome. Science 296, 2225-9 (2002).
78. Loh, P-R. et al. Reference-based phasing using the Haplotype Reference Consortium panel. Nat. Genet. 48, 1443-1448 (2016).
79. Das, S. et al. Next-generation genotype imputation service and methods. Nat. Genet. 48, 1284-1287 (2016).
80. McCarthy, S. et al. A reference panel of 64,976 haplotypes for genotype imputation. Nat. Genet. 48, 1279-83 (2016).
81. Waszak, S. M. et al. Germline elongator mutations in Sonic Hedgehog Medulloblastoma. Nature 580, 396-401 (2020).
82. Yang et al. GCTA: a tool for Genome-wide Complex Trait Analysis. Am J Hum Genet. 88, 76-82 (2011).
83. Yang et al. Genetic variance estimation with imputed variants finds negligible missing heritability for human height and body mass index. Nat Genet. 47, 1114-20 (2015).
84. van de Geijn, B., et al. WASP: allele-specific software for robust molecular quantitative trait locus discovery. Nat Methods 12, 1061-1063 (2015).
85. Stegle, O., Parts, L., Durbin, R. & Winn, J. A Bayesian framework to account for complex non-genetic factors in gene expression levels greatly increases power in eQTL studies. PLoS Comput. Biol. 6, e1000770 (2010).
86. Sinha, A. et al. The Proteogenomic Landscape of Curable Prostate Cancer. Cancer Cell 35, 414-427.e6 (2019).
87. Karczewski, K. J. et al. The mutational constraint spectrum quantified from variation in 141,456 humans. Nature 581, 434-443 (2020).
88. P'ng, C. et al. BPG: Seamless, automated and interactive visualization of scientific data. BMC Bioinformatics 20, 42 (2019).

What is claimed:

1. A method for identifying a TMPRSS2-ERG fusion protein in a subject comprising detecting the presence of a TMPRSS2-ERG fusion protein in a subject genotyped as having one or more single nucleotide polymorphisms selected from the group consisting of rs111620024, rs12500426, rs7679673, rs12653946, rs2837396, and rs2839469.

2. A method for identifying a TMPRSS2-ERG fusion protein in a subject, the method comprising:
(a) genotyping a subject as having one or more single nucleotide polymorphisms selected from the group consisting of rs111620024, rs12500426, rs7679673, rs12653946, rs2837396, and rs2839469; and
(b) detecting the presence of a TMPRSS2-ERG fusion protein in the subject.

3. The method of claim 1 or 2, wherein detecting the presence of the TMPRSS2-ERG fusion protein comprises sequencing nucleic acids from a biological sample from the subject.

4. The method of claim 3, wherein the nucleic acids are tumor DNA.

5. The method of claim 4, wherein the tumor DNA is DNA from one or more prostate cancer cells.

6. The method of claim 3, wherein the nucleic acids are tumor RNA.

7. The method of claim 6, wherein the tumor RNA is RNA from one or more prostate cancer cells.

8. The method of claim 3, wherein the biological sample is a cell free sample.

9. The method of claim 3, wherein the biological sample is a tissue sample.

10. The method of claim 3, wherein the biological sample is a blood sample.

11. The method of claim 3, wherein the biological sample is a saliva sample.

12. The method of claim 3, wherein the biological sample is a urine sample.

13. The method of any of claims 1-12, wherein the one or more single nucleotide polymorphisms comprise rs111620024.

14. The method of any of claims 1-9, wherein the one or more single nucleotide polymorphisms comprise rs12500426.

15. The method of any of claims 1-9, wherein the one or more single nucleotide polymorphisms comprise rs7679673.

16. The method of any of claims 1-9, wherein the one or more single nucleotide polymorphisms comprise rs12653946.

17. The method of any of claims 1-9, wherein the one or more single nucleotide polymorphisms comprise rs2837396.

18. The method of any of claims 1-9, wherein the one or more single nucleotide polymorphisms comprise rs2839469.

19. The method of any of claims 1-18, wherein the one or more single nucleotide polymorphisms are two or more of rs111620024, rs12500426, rs7679673, rs12653946, rs2837396, and rs2839469.

20. The method of any of claims 1-18, wherein the one or more single nucleotide polymorphisms are three or more of rs111620024, rs12500426, rs7679673, rs12653946, rs2837396, and rs2839469.

21. The method of any of claims 1-18, wherein the one or more single nucleotide polymorphisms are four or more of rs111620024, rs12500426, rs7679673, rs12653946, rs2837396, and rs2839469.

22. The method of any of claims 1-18, wherein the one or more single nucleotide polymorphisms are five or more of rs111620024, rs12500426, rs7679673, rs12653946, rs2837396, and rs2839469.

23. The method of any of claims 1-18, wherein the one or more single nucleotide polymorphisms are rs111620024, rs12500426, rs7679673, rs12653946, rs2837396, and rs2839469.

24. The method of any of claims 1-23, wherein the subject is suspected of having cancer.

25. The method of any of claims 1-24, wherein the subject is suspected of having prostate cancer.

26. The method of any of claims 1-25, wherein the subject has not been diagnosed with cancer.

27. The method of any of claims 1-26, wherein the subject has not been diagnosed with prostate cancer.

28. A method for treating a subject for prostate cancer, the method comprising administering an effective amount of a prostate cancer therapy to a subject who (a) has been determined to have a TMPRSS2-ERG fusion protein and (b) has been genotyped as having a single nucleotide polymorphism selected from the group consisting of rs111620024, rs12500426, rs7679673, rs12653946, rs2837396, and rs2839469.

29. A method for treating a subject for prostate cancer, the method comprising
(a) detecting the presence of a TMPRSS2-ERG fusion protein in the subject;
(b) genotyping the subject as having one or more single nucleotide polymorphisms selected from the group consisting of rs111620024, rs12500426, rs7679673, rs12653946, rs2837396, and rs2839469; and
(c) administering an effective amount of a prostate cancer therapy to the subject.

30. The method of claim 28 or 29, wherein the one or more single nucleotide polymorphisms comprise rs111620024.

31. The method of claim 28 or 29, wherein the one or more single nucleotide polymorphisms comprise rs12500426.

32. The method of claim 28 or 29, wherein the one or more single nucleotide polymorphisms comprise rs7679673.

33. The method of claim 28 or 29, wherein the one or more single nucleotide polymorphisms comprise rs12653946.

34. The method of claim 28 or 29, wherein the one or more single nucleotide polymorphisms comprise rs2837396.

35. The method of claim 28 or 29, wherein the one or more single nucleotide polymorphisms comprise rs2839469.

36. The method of any of claims 28-35, wherein the one or more single nucleotide polymorphisms are two or more of rs111620024, rs12500426, rs7679673, rs12653946, rs2837396, and rs2839469.

37. The method of any of claims 28-35, wherein the one or more single nucleotide polymorphisms are three or more of rs111620024, rs12500426, rs7679673, rs12653946, rs2837396, and rs2839469.

38. The method of any of claims 28-35, wherein the one or more single nucleotide polymorphisms are four or more of rs111620024, rs12500426, rs7679673, rs12653946, rs2837396, and rs2839469.

39. The method of any of claims 28-35, wherein the one or more single nucleotide polymorphisms are five or more of rs111620024, rs12500426, rs7679673, rs12653946, rs2837396, and rs2839469.

40. The method of any of claims 28-35, wherein the one or more single nucleotide polymorphisms are rs111620024, rs12500426, rs7679673, rs12653946, rs2837396, and rs2839469.

41. The method of any of claims 28-40, wherein the prostate cancer therapy comprises chemotherapy, hormone therapy, radiotherapy, surgery, immunotherapy, or a combination thereof.

42. A method for identifying a single nucleotide variation in a 5′ UTR of FOXA1 in a subject, the method comprising detecting the presence of a single nucleotide variation in a 5′ UTR of FOXA1 in a subject genotyped as having one or more single nucleotide polymorphisms selected from the group consisting of rs77404504, rs848047, and rs848048.

43. A method for identifying a single nucleotide variation in a 5′ UTR of FOXA1 in a subject, the method comprising:
(a) genotyping a subject as having one or more single nucleotide polymorphisms selected from the group consisting of rs77404504, rs848047, and rs848048; and
(b) detecting the presence of a 5′ UTR of FOXA1 in the subject.

44. The method of claim 42 or 43, wherein detecting the presence of the single nucleotide variation in the 5′ UTR of FOXA1 comprises sequencing nucleic acids from a biological sample from the subject.

45. The method of claim 44, wherein the nucleic acids are tumor DNA.

46. The method of claim 45, wherein the tumor DNA is DNA from one or more prostate cancer cells.

47. The method of claim 44, wherein the nucleic acids are tumor RNA.

48. The method of claim 47, wherein the tumor RNA is RNA from one or more prostate cancer cells.

49. The method of claim 44, wherein the biological sample is a cell free sample.

50. The method of claim 44, wherein the biological sample is a tissue sample.

51. The method of claim 44, wherein the biological sample is a blood sample.

52. The method of claim 44, wherein the biological sample is a saliva sample.

53. The method of claim 44, wherein the biological sample is a urine sample.

54. The method of any of claims 42-53, wherein the one or more single nucleotide polymorphisms comprise rs77404504.

55. The method of any of claims 42-50, wherein the one or more single nucleotide polymorphisms comprise rs848047.

56. The method of any of claims 42-50, wherein the one or more single nucleotide polymorphisms comprise rs848048.

57. The method of any of claims 42-56, wherein the one or more single nucleotide polymorphisms are two or more of rs77404504, rs848047, and rs848048.

58. The method of any of claims 42-56, wherein the one or more single nucleotide polymorphisms are rs77404504, rs848047, and rs848048.

59. The method of any of claims 42-58, wherein the subject is suspected of having cancer.

60. The method of any of claims 42-59, wherein the subject is suspected of having prostate cancer.

61. The method of any of claims 42-60, wherein the subject has not been diagnosed with cancer.

62. The method of any of claims 42-61, wherein the subject has not been diagnosed with prostate cancer.

63. A method for treating a subject for prostate cancer, the method comprising administering an effective amount of a prostate cancer therapy to a subject who (a) has been determined to have a single nucleotide variation in a 5′ UTR of FOXA1 and (b) has been genotyped as having one or more single nucleotide polymorphisms selected from the group consisting of rs77404504, rs848047, and rs848048.

64. A method for treating a subject for prostate cancer, the method comprising
(a) detecting the presence of a single nucleotide variation in a 5′ UTR of FOXA1 in the subject;
(b) genotyping the subject as having one or more single nucleotide polymorphisms selected from the group consisting of rs77404504, rs848047, and rs848048; and
(c) administering an effective amount of a prostate cancer therapy to the subject.

65. The method of claim 63 or 64, wherein the one or more single nucleotide polymorphisms comprise rs77404504.

66. The method of claim 63 or 64, wherein the one or more single nucleotide polymorphisms comprise rs848047.

67. The method of claim 63 or 64, wherein the one or more single nucleotide polymorphisms comprise rs848048.

68. The method of any of claims 63-67, wherein the one or more single nucleotide polymorphisms are two or more of rs77404504, rs848047, and rs848048.

69. The method of any of claims 63-67, wherein the one or more single nucleotide polymorphisms are rs77404504, rs848047, and rs848048.

70. The method of any of claims 63-67, wherein the prostate cancer therapy comprises chemotherapy, hormone therapy, radiotherapy, surgery, immunotherapy, or a combination thereof.

71. A method for assaying for TMPRSS2 in a subject, the method comprising detecting a reduced expression, deletion, or translocation of TMPRSS2 in a subject genotyped as having one or more single nucleotide polymorphisms selected from the group consisting of rs11203152, rs12500426, rs12653946, rs13048402, rs2837396, and rs5759167.

72. A method for assaying for TMPRSS2 in a subject, the method comprising:
(a) genotyping a subject as having one or more single nucleotide polymorphisms selected from the group consisting of rs11203152, rs12500426, rs12653946, rs13048402, rs2837396, and rs5759167; and
(b) detecting a reduced expression, deletion, or translocation of TMPRSS2 in the subject.

73. The method of claim 71 or 72, wherein detecting the reduced expression, deletion, or translocation of TMPRSS2 comprises sequencing nucleic acids from a biological sample from the subject.

74. The method of claim 73, wherein the nucleic acids are tumor DNA.

75. The method of claim 74, wherein the tumor DNA is DNA from one or more prostate cancer cells.

76. The method of claim 73, wherein the nucleic acids are tumor RNA.

77. The method of claim 76, wherein the tumor RNA is RNA from one or more prostate cancer cells.

78. The method of claim 73, wherein the biological sample is a cell free sample.

79. The method of claim 73, wherein the biological sample is a tissue sample.

80. The method of claim 73, wherein the biological sample is a blood sample.

81. The method of claim 73, wherein the biological sample is a saliva sample.

82. The method of claim 73, wherein the biological sample is a urine sample.

83. The method of any of claims 71-82, wherein the one or more single nucleotide polymorphisms comprise rs11203152.

84. The method of any of claims 71-79, wherein the one or more single nucleotide polymorphisms comprise rs12500426.

85. The method of any of claims 71-79, wherein the one or more single nucleotide polymorphisms comprise rs12653946.

86. The method of any of claims 71-79, wherein the one or more single nucleotide polymorphisms comprise rs13048402.

87. The method of any of claims 71-79, wherein the one or more single nucleotide polymorphisms comprise rs2837396.

88. The method of any of claims 71-79, wherein the one or more single nucleotide polymorphisms comprise rs5759167.

89. The method of any of claims 71-88, wherein the one or more single nucleotide polymorphisms are two or more of rs11203152, rs12500426, rs12653946, rs13048402, rs2837396, and rs5759167.

90. The method of any of claims 71-88, wherein the one or more single nucleotide polymorphisms are three or more of rs11203152, rs12500426, rs12653946, rs13048402, rs2837396, and rs5759167.

91. The method of any of claims 71-88, wherein the one or more single nucleotide polymorphisms are four or more of rs11203152, rs12500426, rs12653946, rs13048402, rs2837396, and rs5759167.

92. The method of any of claims 71-88, wherein the one or more single nucleotide polymorphisms are five or more of rs11203152, rs12500426, rs12653946, rs13048402, rs2837396, and rs5759167.

93. The method of any of claims 71-88, wherein the one or more single nucleotide polymorphisms are rs11203152, rs12500426, rs12653946, rs13048402, rs2837396, and rs5759167.

94. The method of any of claims 71-93, wherein the subject is suspected of having cancer.

95. The method of any of claims 71-94, wherein the subject is suspected of having prostate cancer.

96. The method of any of claims 71-95, wherein the subject has not been diagnosed with cancer.

97. The method of any of claims 71-96, wherein the subject has not been diagnosed with prostate cancer.

98. A method for treating a subject for prostate cancer, the method comprising administering an effective amount of a prostate cancer therapy to a subject who (a) has been determined to have a reduced expression, deletion, or translocation of TMPRSS2 and (b) has been genotyped as having one or more single nucleotide polymorphisms selected from the group consisting of rs11203152, rs12500426, rs12653946, rs13048402, rs2837396, and rs5759167.

99. A method for treating a subject for prostate cancer, the method comprising
(a) measuring an expression level of TMPRSS2 in the subject;
(b) genotyping the subject as having one or more single nucleotide polymorphisms selected from the group consisting of rs11203152, rs12500426, rs12653946, rs13048402, rs2837396, and rs5759167; and
(c) administering an effective amount of a prostate cancer therapy to the subject.

100. The method of claim 98 or 99, wherein the one or more single nucleotide polymorphisms comprise rs11203152.

101. The method of claim 98 or 99, wherein the one or more single nucleotide polymorphisms comprise rs12500426.

102. The method of claim 98 or 99, wherein the one or more single nucleotide polymorphisms comprise rs12653946.

103. The method of claim 98 or 99, wherein the one or more single nucleotide polymorphisms comprise rs13048402.

104. The method of claim 98 or 99, wherein the one or more single nucleotide polymorphisms comprise rs2837396.

105. The method of claim 98 or 99, wherein the one or more single nucleotide polymorphisms comprise rs5759167.

106. The method of any of claims 98-105, wherein the one or more single nucleotide polymorphisms are two or more of rs11203152, rs12500426, rs12653946, rs13048402, rs2837396, and rs5759167.

107. The method of any of claims 98-105, wherein the one or more single nucleotide polymorphisms are three or more of rs11203152, rs12500426, rs12653946, rs13048402, rs2837396, and rs5759167.

108. The method of any of claims 98-105, wherein the one or more single nucleotide polymorphisms are four or more of rs11203152, rs12500426, rs12653946, rs13048402, rs2837396, and rs5759167.

109. The method of any of claims 98-105, wherein the one or more single nucleotide polymorphisms are five or more of rs11203152, rs12500426, rs12653946, rs13048402, rs2837396, and rs5759167.

110. The method of any of claims 98-105, wherein the one or more single nucleotide polymorphisms are rs11203152, rs12500426, rs12653946, rs13048402, rs2837396, and rs5759167.

111. The method of any of claims 99-106, wherein the prostate cancer therapy comprises chemotherapy, hormone therapy, radiotherapy, surgery, immunotherapy, or a combination thereof.

112. A method for assaying for CDKN1B in a subject, the method comprising detecting a reduced expression level or deletion of CDKN1B in a subject genotyped as having one or more single nucleotide polymorphisms selected from the group consisting of rs12817741, rs12824766, rs141393446, rs141853059, rs57526507, and rs61915608.

113. A method for assaying for CDKN1B in a subject, the method comprising:
(a) genotyping a subject as having one or more single nucleotide polymorphisms selected from the group consisting of rs12817741, rs12824766, rs141393446, rs141853059, rs57526507, and rs61915608; and
(b) detecting a reduced expression level or deletion of CDKN1B in the subject.

114. The method of claim 112 or 113, wherein detecting the reduced expression level or deletion CDKN1B comprises sequencing nucleic acids from a biological sample from the subject.

115. The method of claim 114, wherein the nucleic acids are tumor DNA.

116. The method of claim 115, wherein the tumor DNA is DNA from one or more prostate cancer cells.

117. The method of claim 114, wherein the nucleic acids are tumor RNA.

118. The method of claim 117, wherein the tumor RNA is RNA from one or more prostate cancer cells.

119. The method of claim 114, wherein the biological sample is a cell free sample.

120. The method of claim 114, wherein the biological sample is a tissue sample.

121. The method of claim 114, wherein the biological sample is a blood sample.

122. The method of claim 114, wherein the biological sample is a saliva sample.

123. The method of claim 114, wherein the biological sample is a urine sample.

124. The method of any of claims 112-120, wherein the one or more single nucleotide polymorphisms comprise rs12817741.

125. The method of any of claims 112-120, wherein the one or more single nucleotide polymorphisms comprise rs12824766.

126. The method of any of claims 112-120, wherein the one or more single nucleotide polymorphisms comprise rs141393446.

127. The method of any of claims 112-120, wherein the one or more single nucleotide polymorphisms comprise rs141853059.

128. The method of any of claims 112-120, wherein the one or more single nucleotide polymorphisms comprise rs57526507.

129. The method of any of claims 112-120, wherein the one or more single nucleotide polymorphisms comprise rs61915608.

130. The method of any of claims 112-129, wherein the one or more single nucleotide polymorphisms are two or more of rs12817741, rs12824766, rs141393446, rs141853059, rs57526507, and rs61915608.

131. The method of any of claims 112-129, wherein the one or more single nucleotide polymorphisms are three or more of rs12817741, rs12824766, rs141393446, rs141853059, rs57526507, and rs61915608.

132. The method of any of claims 112-129, wherein the one or more single nucleotide polymorphisms are four or more of rs12817741, rs12824766, rs141393446, rs141853059, rs57526507, and rs61915608.

133. The method of any of claims 112-129, wherein the one or more single nucleotide polymorphisms are five or more of rs12817741, rs12824766, rs141393446, rs141853059, rs57526507, and rs61915608.

134. The method of any of claims 112-129, wherein the one or more single nucleotide polymorphisms are rs12817741, rs12824766, rs141393446, rs141853059, rs57526507, and rs61915608.

135. The method of any of claims 112-134, wherein the subject is suspected of having cancer.

136. The method of any of claims 112-135, wherein the subject is suspected of having prostate cancer.

137. The method of any of claims 112-136, wherein the subject has not been diagnosed with prostate cancer.

138. The method of any of claims 112-137, wherein the subject has not been diagnosed with prostate cancer.

139. A method for treating a subject for prostate cancer, the method comprising administering an effective amount of a prostate cancer therapy to a subject who (a) has been determined to have a reduced expression or deletion of CDKN1B and (b) has been genotyped as having one or more single nucleotide polymorphisms selected from the group consisting of rs12817741, rs12824766, rs141393446, rs141853059, rs57526507, and rs61915608.

140. A method for treating a subject for prostate cancer, the method comprising
(a) measuring an expression level of CDKN1B in the subject;
(b) genotyping the subject as having one or more single nucleotide polymorphisms selected from the group consisting of rs12817741, rs12824766, rs141393446, rs141853059, rs57526507, and rs61915608; and
(c) administering an effective amount of a prostate cancer therapy to the subject.

141. The method of claim 139 or 140, wherein the one or more single nucleotide polymorphisms comprise rs12817741.

142. The method of claim 139 or 140, wherein the one or more single nucleotide polymorphisms comprise rs12824766.

143. The method of claim 139 or 140, wherein the one or more single nucleotide polymorphisms comprise rs141393446.

144. The method of claim 139 or 140, wherein the one or more single nucleotide polymorphisms comprise rs141853059.

145. The method of claim 139 or 140, wherein the one or more single nucleotide polymorphisms comprise rs57526507.

146. The method of claim 139 or 140, wherein the one or more single nucleotide polymorphisms comprise rs61915608.

147. The method of any of claims 139-146, wherein the one or more single nucleotide polymorphisms are two or more of rs12817741, rs12824766, rs141393446, rs141853059, rs57526507, and rs61915608.

148. The method of any of claims 139-146, wherein the one or more single nucleotide polymorphisms are three or more of rs12817741, rs12824766, rs141393446, rs141853059, rs57526507, and rs61915608.

149. The method of any of claims 139-146, wherein the one or more single nucleotide polymorphisms are four or more of rs12817741, rs12824766, rs141393446, rs141853059, rs57526507, and rs61915608.

150. The method of any of claims 139-146, wherein the one or more single nucleotide polymorphisms are five or more of rs12817741, rs12824766, rs141393446, rs141853059, rs57526507, and rs61915608.

151. The method of any of claims 139-146, wherein the one or more single nucleotide polymorphisms are rs12817741, rs12824766, rs141393446, rs141853059, rs57526507, and rs61915608.

152. The method of any of claims 140-151, wherein the prostate cancer therapy comprises chemotherapy, hormone therapy, radiotherapy, surgery, immunotherapy, or a combination thereof.

153. A method for identifying one or more genetic abnormalities in a subject, the method comprising:
(a) detecting the presence of a TMPRSS2-ERG fusion protein in a subject genotyped as having one or more single nucleotide polymorphisms selected from the group consisting of rs111620024, rs12500426, rs7679673, rs12653946, rs2837396, and rs2839469;
(b) detecting the presence of a single nucleotide variation in a 5′ UTR of FOXA1 in a subject genotyped as having one or more single nucleotide polymorphisms selected from the group consisting of rs77404504, rs848047, and rs848048;
(c) detecting a reduced expression level, deletion, or translocation of TMPRSS2 in a subject genotyped as having one or more single nucleotide polymorphisms selected from the group consisting of rs11203152, rs12500426, rs12653946, rs13048402, rs2837396, and rs5759167; or
(d) detecting a reduced expression level or deletion of CDKN1B in a subject genotyped as having one or more single nucleotide polymorphisms selected from the group consisting of rs12817741, rs12824766, rs141393446, rs141853059, rs57526507, and rs61915608.

154. The method of claim 153, wherein the method comprises two or more of (a), (b), (c), and (d).

155. The method of claim 153, wherein the method comprises three or more of (a), (b), (c), and (d).

156. The method of claim 153, wherein the method comprises (a), (b), (c), and (d).

157. The method of any of claims 145-156, wherein the subject is suspected of having cancer.

158. The method of any of claims 153-157, wherein the subject is suspected of having prostate cancer.

159. The method of any of claims 153-158, wherein the subject has not been diagnosed with cancer.

160. The method of any of claims 153-159, wherein the subject has not been diagnosed with prostate cancer.

161. A method for identifying one or more genetic abnormalities in a subject, the method comprising:
(a) (i) genotyping a subject as having one or more single nucleotide polymorphisms selected from the group consisting of rs111620024, rs12500426, rs7679673, rs12653946, rs2837396, and rs2839469, and (ii) detecting the presence of a TMPRSS2-ERG fusion protein in the subject;
(b) (i) genotyping a subject as having one or more single nucleotide polymorphisms selected from the group consisting of rs77404504, rs848047, and rs848048, and (ii) detecting the presence of a 5′ UTR of FOXA1 in the subject;
(c) (i) genotyping a subject as having one or more single nucleotide polymorphisms selected from the group consisting of rs11203152, rs12500426, rs12653946, rs13048402, rs2837396, and rs5759167, and (ii) detecting a reduced expression level, deletion, or translocation of TMPRSS2 in the subject; or
(d) (i) genotyping a subject as having one or more single nucleotide polymorphisms selected from the group consisting of rs12817741, rs12824766, rs141393446, rs141853059, rs57526507, and rs61915608, and (ii) detecting a reduced expression level or deletion of CDKN1B in the subject.

162. The method of claim 161, wherein the method comprises two or more of (a), (b), (c), and (d).

163. The method of claim 161, wherein the method comprises three or more of (a), (b), (c), and (d).

164. The method of claim 161, wherein the method comprises (a), (b), (c), and (d).

165. The method of any of claims 161-164, wherein the subject is suspected of having cancer.

166. The method of any of claims 161-165, wherein the subject is suspected of having prostate cancer.

167. The method of any of claims 161-166, wherein the subject has not been diagnosed with cancer.

168. The method of any of claims 161-167, wherein the subject has not been diagnosed with prostate cancer.

169. A method for treating a subject for prostate cancer, the method comprising administering an effective amount of a prostate cancer therapy to a subject who:
(a) has been determined to have a TMPRSS2-ERG fusion protein and has been genotyped as having one or more single nucleotide polymorphisms selected from the group consisting of rs111620024, rs12500426, rs7679673, rs12653946, rs2837396, and rs2839469;
(b) has been determined to have a single nucleotide variation in a 5′ UTR of FOXA1 and has been genotyped as having one or more single nucleotide polymorphisms selected from the group consisting of rs77404504, rs848047, and rs848048;
(c) has been determined to have reduced expression, a deletion, or a translocation of TMPRSS2 and has been genotyped as having one or more single nucleotide polymorphisms selected from the group consisting of rs11203152, rs12500426, rs12653946, rs13048402, rs2837396, and rs5759167; or
(d) has been determined to have reduced expression or a deletion of CDKN1B and has been genotyped as having one or more single nucleotide polymorphisms selected from the group consisting of rs12817741, rs12824766, rs141393446, rs141853059, rs57526507, and rs61915608.

170. The method of claim 169, wherein the method comprises two or more of (a), (b), (c), and (d).

171. The method of claim 169, wherein the method comprises three or more of (a), (b), (c), and (d).

172. The method of claim 169, wherein the method comprises (a), (b), (c), and (d).

173. The method of any of claims 169-172, wherein the prostate cancer therapy comprises chemotherapy, hormone therapy, radiotherapy, surgery, immunotherapy, or a combination thereof.

174. A method for diagnosing a subject as having prostate cancer, the method comprising:
(a) detecting the presence of a TMPRSS2-ERG fusion protein in a subject genotyped as having one or more single nucleotide polymorphisms selected from the group consisting of rs111620024, rs12500426, rs7679673, rs12653946, rs2837396, and rs2839469;
(b) detecting the presence of a single nucleotide variation in a 5′ UTR of FOXA1 in a subject genotyped as having one or more single nucleotide polymorphisms selected from the group consisting of rs77404504, rs848047, and rs848048;
(c) measuring a reduced expression level, deletion, or translocation of TMPRSS2 in a subject genotyped as having one or more single nucleotide polymorphisms selected from the group consisting of rs11203152, rs12500426, rs12653946, rs13048402, rs2837396, and rs5759167; or
(d) measuring a reduced expression level or deletion of CDKN1B in a subject genotyped as having one or more single nucleotide polymorphisms selected from the group consisting of rs12817741, rs12824766, rs141393446, rs141853059, rs57526507, and rs61915608.

175. The method of claim 174, wherein the method comprises two or more of (a), (b), (c), and (d).

176. The method of claim 174, wherein the method comprises three or more of (a), (b), (c), and (d).

177. The method of claim 174, wherein the method comprises (a), (b), (c), and (d).
